(19) United States
(12) Patent Application Publication
(10) Pub. No.: US 2026/0200921 A1
(43) Pub. Date: Jul. 16, 2026

(54) SUBSTITUTED AMINOPYRIDINE COMPOUNDS AS AKT INHIBITORS

(71) Applicant: Atavistik Bio, Inc., Cambridge, MA (US)

(72) Inventors: Maria Jesus Blanco-Pillado, Arlington, MA (US); Aaron Robert Coffin, Everett, MA (US)

(21) Appl. No.: 19/561,334

(22) Filed: Mar. 9, 2026

Related U.S. Application Data
(63) Continuation of application No. PCT/US2024/046621, filed on Sep. 13, 2024.
(60) Provisional application No. 63/538,649, filed on Sep. 15, 2023.

(51) Int. Cl. C07D 471/04; A61K 31/4439; A61K 31/444; A61K 31/4545; C07D 401/14

(52) U.S. Cl. CPC C07D 471/04; A61K 31/4439; A61K 31/444; A61K 31/4545; C07D 401/14

(57) ABSTRACT

The invention provides aminopyridinyl imidazo[4,5-b]pyridine of formula (I), pharmaceutical compositions, and their use in treating disease, such as cancer.

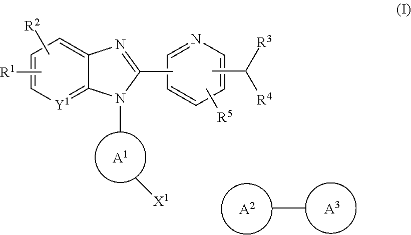

### CROSS-REFERENCE TO RELATED APPLICATION

This application claims priority to and the benefit of U.S. Provisional Patent Application No. 63/538,649, filed Sep. 15, 2023, the content of which is hereby incorporated by reference in its entirety.

### BRIEF SUMMARY

The invention provides aminopyridinyl imidazo[4,5-b]pyridine and related compounds, pharmaceutical compositions, and their use in treating disease, such as cancer.

### BACKGROUND

Cancer continues to be a significant health problem despite the substantial research efforts and scientific advances reported in the literature for treating this disease. Solid tumors, such as prostate cancer, colon, rectum, skin cancer, breast cancer, and lung cancer remain highly prevalent among the world population. Existing therapies for treating cancer include localized therapies, such as surgery, radiation therapy, cryotherapy, and systemic therapies (e.g., chemotherapy, hormonal therapy, immune therapy, and targeted therapy) used alone or in combination. Support therapies are also used in some contexts, where supportive therapies are additional treatments that do not directly treat cancer but are used to reduce side effects and address patient quality of life. However, current treatment options for cancer are not effective for all patients and/or can have substantial adverse side effects. New therapies are needed to address this unmet need in cancer therapy.

The AKT protein family, which members are also named as protein kinase B (PKB), are important mediators of growth factor induced cellular signaling. See, for example, Manning and Toker. Cell 169. Apr. 20, 2017. The AKT protein family belongs to the larger serine/threonine kinase family regulated by the phosphoinositide 3-kinase (PI3K) pathway—a critical signal transduction system linking oncogenes and multiple receptor classes to many essential cellular functions. Class I PI3K phosphorylates the 3′ hydroxyl of the inositol head group of phosphoinositides (PtdIns), resulting in the production of the lipid second messengers PtdIns-3,4-P₂ (PIP2) and PtdIns3,4,5-P3 (PIP3). PIP3 recruits AKT to the plasma membrane where it is activated and triggers a signaling cascade impinging upon pathways important for cancer cell growth.

In humans, there are three AKT isoforms encoded by three separate genes: AKT1, AKT2 and AKT3. These genes encode for serine/threonine kinases that are activated by growth factors in a phosphoinositide 3-kinase (PI3K) dependent manner. AKT1 regulates cell growth and survival and is expressed in several tissues. AKT2 is an important signaling molecule in the insulin signaling pathway and is required to induce glucose transport in certain tissues. The role of AKT3 is less known, and it appears to be predominantly expressed in the brain.

Among the three AKT isoforms, AKT1 is most frequently hyperactivated in tumors. The hyperactivation of AKT1 in tumors underscores the important role AKT1 plays in tumorigenesis and cancer cell proliferation and survival (Bhattarai et al. Nature Communications volume 13, Article number: 2111, 2022). AKT1 signaling promotes tumor cell growth by modulating multiple different effectors. For example, AKT1 regulates protein synthesis via the mTORC1 pathway, cellular metabolism via GSK3-beta pathway and multiple metabolic enzymes, and cell profileration and survival via the FOXO family of transcription factors. See, for example, Hoxhaj and Manning, Nat Rev Cancer. 2020 February; 20(2): 74-88.

The most common mutation in AKT1 is a point mutation converting a glutamic acid of residue 17 to a lysine (E17K). The E17K mutation [Carpten et al. Nature 448, 2007, 439-444] in the Pleckstrin Homology Domain (PHD) of AKT1 leads to constitutive association with the plasma membrane via ectopic binding to PIP2 and increased affinity for PIP3. Consequently, this deregulated recruitment of AKT1 to the plasma membrane causes constitutive activation of AKT1 signaling and has been shown to be a recurrent mutation in breast, endometrial, and other cancers.

The oncogenic properties of the E17K single point mutation in AKT1 make it a target for inhibition for anti-cancer agents. In addition to cancer, somatic mutation of AKT1 to the E17K variant during embryonic development leads to the overgrowth disease Proteus Syndrome (N Engl J Med. 2011 Aug. 18; 365(7): 611-619). Proteus syndrome is characterized by the overgrowth of skin, connective tissue, brain, and other tissues. Patients with this disease also have an increased risk of premature death due to vascular deformities.

New compounds that inhibit AKT1 are needed and would provide a medical benefit to patients suffering from disorders associated with AKT1 activity. The present invention addresses the foregoing needs and provides other related advantages.

### SUMMARY

The invention provides aminopyridinyl imidazo[4,5-b]pyridine and related compounds, pharmaceutical compositions, and their use in treating disease, such as cancer. In particular, one aspect of the invention provides a collection of aminopyridinyl imidazo[4,5-b]pyridine and related compounds, such as a compound represented by Formula I:

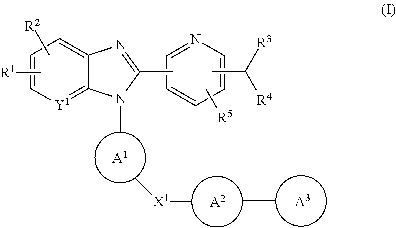

or a pharmaceutically acceptable salt thereof, where the variables are as defined in the detailed description. Further description of additional collections of aminopyridinyl imidazo[4,5-b]pyridine and related compounds are described in the detailed description. The compounds may be part of a pharmaceutical composition comprising a pharmaceutically acceptable carrier.

Another aspect of the invention provides a method of treating a disease or disorder associated with aberrant AKT1 signaling. The method comprises administering to a subject in need thereof a therapeutically effective amount of a compound described herein, such as a compound of Formula I, to treat the disease or disorder, as further described in the detailed description.

Another aspect of the invention provides a method of treating cancer. The method comprises administering to a subject in need thereof a therapeutically effective amount of a compound described herein, such as a compound of Formula I, to treat the cancer, as further described in the detailed description. In certain embodiments, the cancer has a AKT1 mutation.

A method of treating a disease or disorder associated with active PI3K signaling. The method comprises administering to a subject in need thereof a therapeutically effective amount of a compound described herein, such as a compound of Formula I, to treat the disease or disorder.

Another aspect of the invention provides a method inhibiting AKT1 activity. The method comprises contacting an AKT1 with an effective amount of a compound described herein, such as a compound of Formula I, to thereby inhibit the AKT1 activity, as further described in the detailed description.

### DETAILED DESCRIPTION

The invention provides aminopyridinyl imidazo[4,5-b]pyridine and related compounds, pharmaceutical compositions, and their use in treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements. CAS version. Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith. M. B, and March. J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In certain embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In certain embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In certain embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

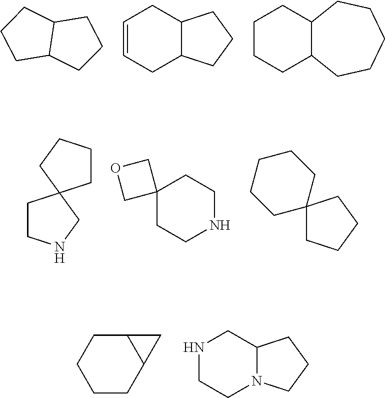

Exemplary bridged bicyclics include:

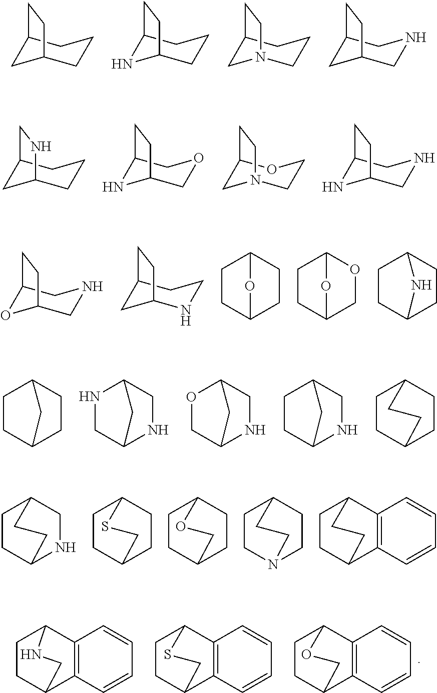

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated.” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The terms “halogen” and “halo are used interchangeably and mean F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

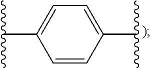 [SMILES: *c1ccc(*)cc1]

“phenylene” is a trivalent phenyl group when it has three groups attached to it

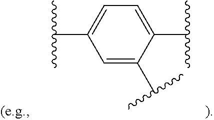 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by one or more oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it. The term “oxo-heterocyclylene” refers to a multivalent oxo-heterocyclyl group having the appropriate number of open valences to account for groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂;
—N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}—, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J, of Pharmaceutics (1986) 33 201-217; Anderson et al., The Practice of Medicinal Chemistry (1996), Academic Press, New York; and in The Orange Book (Food & Drug Administration. Washington. D.C, on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, the replacement of a carbon by a ¹³C- or ⁴C-enriched carbon, or the replacement of a fluorine by a ¹⁸F-enriched fluorine are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterosubstituted Aminopyridinyl Imidazo[4,5-b]pyridine & Related Compounds

One aspect of the invention provides aminopyridinyl imidazo[4,5-b]pyridine and related compounds. The compounds may be used in the pharmaceutical compositions and therapeutic methods described herein. Exemplary compounds are described in the following sections, along with exemplary procedures for making the compounds. One aspect of the invention provides a compound represented by Formula I:

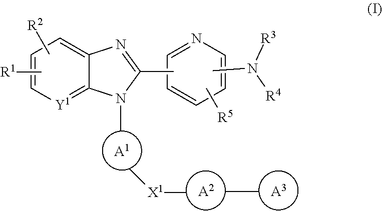

or a pharmaceutically acceptable salt thereof; wherein:
R¹ is phenyl or 6-membered heteroaryl containing 1 or 2 heteroatoms selected from nitrogen, wherein the phenyl and heteroaryl are each substituted with 0, 1, or 2 substituents independently selected from halo or C₁₋₄ alkyl;
R², R³, R⁴, R⁵, and R⁹ are independently hydrogen or C₁₋₄ alkyl;
R⁶ and R⁷ are independently halo or C₁₋₄ alkyl;
R⁸ represents independently for each occurrence hydroxyl, oxo, C₁₋₄ alkoxyl, C₁₋₄ alkyl, C₁₋₄ haloalkyl, cyano, —(C₀₋₄ alkylene)-CO₂R⁹, —(C₀₋₄ alkylene)-C(O)N(R¹⁰)(R¹¹), —(C₀₋₄ alkylene)-N(R¹⁰)C(O)R¹², or phenyl, wherein the phenyl is substituted with 0, 1, or 2 substituents independently selected from the group consisting of halo and C₁₋₄ alkyl;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² is C₁₋₆ alkyl or C₃₋₆ cycloalkyl;
A¹ is phenylene or pyridinylene, each of which is substituted with 0 or 1 occurrence of R⁶;
A² is phenylene, C₃₋₆ cycloalkyl, or pyridinylene, each of which is substituted with 0 or 1 occurrence of R⁷;
A³ is a 5-membered heteroaryl or heterocyclyl containing 2, 3, or 4 heteroatoms selected from nitrogen and sulphur, wherein the heteroaryl or heterocyclyl is substituted with n occurrences of R⁸;
X¹ is —(C₀₋₃ alkylene)-N(R⁹)C(O)-Ψ, —(C₀₋₃ alkylene)-N(R⁹)-Ψ, -(3-6 membered saturated heterocyclylene containing 1 or 2 heteroatoms selected from nitrogen)-(C₀₋₃ alkylene)-Ψ, or -(3-5 membered saturated heterocyclylene containing 1 or 2 heteroatoms selected from nitrogen)-C(O)-Ψ; wherein Ψ is a bond to A²;
Y¹ is N or —C(H)—; and
   n is 0, 1, or 2

One aspect of the invention provides a compound represented by Formula I:

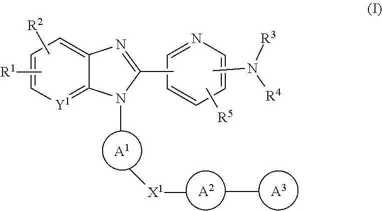

or a pharmaceutically acceptable salt thereof; wherein:
R¹ is phenyl or 6-membered heteroaryl containing 1 or 2 heteroatoms selected from nitrogen, wherein the phenyl and heteroaryl are each substituted with 0, 1, or 2 substituents independently selected from halo or C₁₋₄ alkyl;
R², R³, R⁴, R⁵, and R⁹ are independently hydrogen or C₁₋₄ alkyl;
R⁶ and R⁷ are independently halo or C₁₋₄ alkyl;
R⁸ represents independently for each occurrence hydroxyl, C₁₋₄ alkoxyl, C₁₋₄ alkyl, C₁₋₄ haloalkyl, cyano, —(C₀₋₄ alkylene)-CO₂R⁹, —(C₀₋₄ alkylene)-C(O)N(R¹⁰)(R¹¹), —(C₀₋₄ alkylene)-N(R¹⁰)C(O)R¹², or phenyl, wherein the phenyl is substituted with 0, 1, or 2 substituents independently selected from the group consisting of halo and C₁₋₄ alkyl;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² is C₁₋₆ alkyl or C₃₋₆ cycloalkyl;
A¹ is phenylene or pyridinylene, each of which is substituted with 0 or 1 occurrence of R⁶;
A² is phenylene or pyridinylene, each of which is substituted with 0 or 1 occurrence of R⁷;
A³ is a 5-membered heteroaryl containing 2, 3, or 4 heteroatoms selected from nitrogen, wherein the heteroaryl is substituted with n occurrences of R⁸;
X¹ is —(C₀₋₃ alkylene)-N(R⁹)C(O)-Ψ or -(3-5 membered saturated heterocyclylene containing 1 or 2 heteroatoms selected from nitrogen)-C(O)-Ψ; wherein Ψ is a bond to A²;
Y¹ is N or —C(H)—; and
n is 0, 1, or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As defined generally above, R¹ is phenyl or 6-membered heteroaryl containing 1 or 2 heteroatoms selected from nitrogen, wherein the phenyl and heteroaryl are each substituted with 0, 1, or 2 substituents independently selected from halo or C₁₋₄ alkyl. In certain embodiments, R¹ is phenyl substituted with 0, 1, or 2 substituents independently selected from halo or C₁₋₄ alkyl. In certain embodiments, R¹ is 6-membered heteroaryl containing 1 or 2 heteroatoms selected from nitrogen, wherein the heteroaryl is substituted with 0, 1, or 2 substituents independently selected from halo or C₁₋₄ alkyl. In certain embodiments, R¹ is phenyl substituted with 1-2 substituents independently selected from halo or C₁₋₄ alkyl. In certain embodiments, R¹ is 6-membered heteroaryl containing 1 or 2 heteroatoms selected from nitrogen, wherein the heteroaryl is substituted with 1-2 substituents independently selected from halo or C₁₋₄ alkyl. In certain embodiments, R¹ is phenyl. In certain embodiments, R¹ is selected from the groups depicted in the compounds in Table 1, below.

As defined generally above, R², R³, R¹, R⁵, and R⁹ are independently hydrogen or C₁₋₄ alkyl. In certain embodiments, R² is hydrogen or C₁₋₄ alkyl. In certain embodiments, R³ is hydrogen or C₁₋₄ alkyl. In certain embodiments, R⁴ is hydrogen or C₁₋₄ alkyl. In certain embodiments, R⁵ is hydrogen or C₁₋₄ alkyl. In certain embodiments, R⁹ is hydrogen or C₁₋₄ alkyl. In certain embodiments. R² is C₁₋₄ alkyl. In certain embodiments. R³ is C₁₋₄ alkyl. In certain embodiments, R⁴ is C₁₋₄ alkyl. In certain embodiments, R⁵ is C₁₋₄ alkyl. In certain embodiments, R⁹ is C₁₋₄ alkyl. In certain embodiments, R², R³, R⁴, R⁵, and R⁹ are hydrogen. In certain embodiments, R², R³, R⁴, R⁵, and R⁹ are C₁₋₄ alkyl.

In certain embodiments, R² is hydrogen. In certain embodiments, R³ is hydrogen. In certain embodiments, R⁴ is hydrogen. In certain embodiments, R⁵ is hydrogen. In certain embodiments, R⁹ is hydrogen.

In certain embodiments, R² is selected from the groups depicted in the compounds in Table 1, below. In certain embodiments, R³ is selected from the groups depicted in the compounds in Table 1, below. In certain embodiments, R⁴ is selected from the groups depicted in the compounds in Table 1, below. In certain embodiments, R⁵ is selected from the groups depicted in the compounds in Table 1, below. In certain embodiments, R⁹ is selected from the groups depicted in the compounds in Table 1, below.

As defined generally above, R⁶ and R⁷ are independently halo or C₁₋₄ alkyl. In certain embodiments, R⁶ is halo or C₁₋₄ alkyl. In certain embodiments. R⁶ is halo. In certain embodiments, R⁶ is C₁₋₄ alkyl. In certain embodiments, R⁷ is halo or C₁₋₄ alkyl. In certain embodiments, R⁷ is halo. In certain embodiments, R⁷ is C₁₋₄ alkyl. In certain embodiments, R⁶ and R⁷ are halo. In certain embodiments R⁶ and R⁷ are C₁₋₄ alkyl. In certain embodiments, R⁷ is fluoro. In certain embodiments, R⁶ is selected from the groups depicted in the compounds in Table 1, below. In certain embodiments, R⁷ is selected from the groups depicted in the compounds in Table 1, below.

As defined generally above, R⁸ represents independently for each occurrence hydroxyl, oxo, C₁₋₄ alkoxyl, C₁₋₄ alkyl, C₁₋₄ haloalkyl, cyano, —(C₀₋₄ alkylene)-CO₂R⁹, —(C₀₋₄ alkylene)-C(O)N(R⁵)(R¹¹), —(C₀₋₄ alkylene)-N(R¹¹)C(O)R¹², or phenyl, wherein the phenyl is substituted with 0, 1, or 2 substituents independently selected from the group consisting of halo and C₁₋₄ alkyl. In certain embodiments, R⁸ represents independently for each occurrence hydroxyl, C₁₋₄ alkoxyl. C₁₋₄ alkyl, C₁₋₄ haloalkyl, cyano, —(C₀₋₄ alkylene)-CO₂R⁹, —(C₀₋₄ alkylene)-C(O)N(R¹⁰)(R¹¹), —(C₀₋₄ alkylene)-N(R¹⁰)C(O)R¹², or phenyl, wherein the phenyl is substituted with 0, 1, or 2 substituents independently selected from the group consisting of halo and C₁₋₄ alkyl. In certain embodiments, R⁸ represents for each occurrence hydroxyl. In certain embodiments, R⁸ represents for each occurrence C₁₋₄ alkoxyl. In certain embodiments, R⁸ represents for each occurrence C₁₋₄ alkyl. In certain embodiments. R⁸ represents for each occurrence C₁₋₄ haloalkyl. In certain embodiments, R⁸ represents for each occurrence cyano. In certain embodiments, R⁸ represents for each occurrence —(C₀₋₄ alkylene)-CO₂R⁹. In certain embodiments, R⁸ represents for each occurrence —(C₀₋₄ alkylene)-C(O)N(R¹⁰)(R¹¹). In certain embodiments, R⁸ represents for each occurrence —(C₀₋₄ alkylene)-N(R¹⁰)C(O)R¹². In certain embodiments. R⁸ represents for each occurrence phenyl, wherein the phenyl is substituted with 0, 1, or 2 substituents independently selected from the group consisting of halo and C₁₋₄ alkyl; In certain embodiments, R⁸ represents for each occurrence phenyl, wherein the phenyl is independently substituted with 1-2 substituents independently selected from the group consisting of halo and C₁₋₄ alkyl. In certain embodiments. R⁸ represents for each occurrence phenyl, wherein the phenyl is substituted with 1-2 substituents of halo. In certain embodiments, R⁸ represents for each occurrence phenyl, wherein the phenyl is substituted with 1-2 substituents of C₁₋₄ alkyl.

In certain embodiments, R⁸ is hydroxyl. In certain embodiments, R⁸ is oxo. In certain embodiments, R⁸ is C₁₋₄ alkoxyl. In certain embodiments. R⁸ is C₁₋₄ alkyl. In certain embodiments, R⁸ is C₁₋₄ haloalkyl. In certain embodiments. R⁸ is cyano. In certain embodiments, R⁸ is —(C₀₋₄ alkylene)-CO₂R⁹. In certain embodiments, R⁸ is —(C₀₋₄ alkylene)-C(O)N(R¹⁰)(R¹¹). In certain embodiments, R⁸ is —(C₀₋₄ alkylene)-N(R¹⁰)C(O)R¹². In certain embodiments, R⁸ is phenyl, wherein the phenyl is substituted with 0, 1, or 2 substituents independently selected from the group consisting of halo and C₁₋₄ alkyl; In certain embodiments, R⁸ is phenyl, wherein the phenyl is independently substituted with 1-2 substituents independently selected from the group consisting of halo and C₁₋₄ alkyl. In certain embodiments, R⁸ is phenyl, wherein the phenyl is substituted with 1-2 substituents of halo. In certain embodiments, R⁸ is phenyl, wherein the phenyl is substituted with 1-2 substituents of C₁₋₄ alkyl.

In certain embodiments, R⁸ represents independently for each occurrence hydroxyl, C₁₋₄ alkoxyl, or C₁₋₄ alkyl. In certain embodiments, R⁸ represents independently for each occurrence hydroxyl, oxo, C₁₋₄ alkoxyl, or C₁₋₄ alkyl. In certain embodiments, R⁸ represents independently for each occurrence hydroxyl or C₁₋₄ alkyl. In certain embodiments, R⁸ represents independently for each occurrence hydroxyl or methyl. In certain embodiments, R⁸ represents independently for each occurrence C₁₋₄ alkoxyl or C₁₋₄ alkyl. In certain embodiments, R⁸ represents independently for each occurrence methoxy or methyl. In certain embodiments, R⁸ represents independently for each occurrence C₁₋₄ alkyl, C₁₋₄ haloalkyl, or cyano. In certain embodiments, R⁸ represents independently for each occurrence hydroxyl or —(C₀₋₄ alkylene)-CO₂R⁹.

In certain embodiments, R⁸ represents independently for each occurrence hydroxyl or —CH₂CO₂CH₃. In certain embodiments, R⁸ is methyl. In certain embodiments, R⁸ is methoxy. In certain embodiments, R⁸ is —CO₂H.

In certain embodiments, R⁸ is selected from the groups depicted in the compounds in Table 1, below.

As defined generally above, R¹⁰ and R¹¹ each represent independently for each occurrence hydrogen or C₁₋₄ alkyl, or R¹⁰ and Rn are taken together with the nitrogen atom to which they are attached to form a 3-7 membered ring. In certain embodiments, R¹⁰ and R¹¹ are hydrogen. In certain embodiments, R¹⁰ and R¹¹ C₁₋₄ alkyl. In certain embodiments, R¹⁰ is hydrogen. In certain embodiments, R¹¹ is hydrogen. In certain embodiments, R¹⁰ is C₁₋₄ alkyl. In certain embodiments, R¹¹ is C₁₋₄ alkyl. In certain embodiments, R¹⁰ and R¹¹ are taken together with the nitrogen atom to which they are attached to form a 3-7 membered ring. In certain embodiments, R¹⁰ is selected from the groups depicted in the compounds in Table 1, below. In certain embodiments, R¹¹ is selected from the groups depicted in the compounds in Table 1, below.

As defined generally above, R¹² is C₁₋₆ alkyl or C₃₋₆ cycloalkyl. In certain embodiments, R¹² is C₁₋₆ alkyl. In certain embodiments, R¹² is C₃₋₆ cycloalkyl. In certain embodiments, R¹² is selected from the groups depicted in the compounds in Table 1, below.

As defined generally above, A¹ is phenylene or pyridinylene, each of which is substituted with 0 or 1 occurrence of R⁶. In certain embodiments, A¹ is phenylene substituted with 0 or 1 occurrence of R⁶. In certain embodiments, A¹ is pyridinylene substituted with 0 or 1 occurrence of R⁶. In certain embodiments, A¹ is phenylene substituted with 1 occurrence of R⁶. In certain embodiments, A¹ is pyridinylene substituted with 1 occurrence of R⁶. In certain embodiments, A¹ is phenylene. In certain embodiments, A¹ is pyridinylene. In certain embodiments, A¹ is selected from the groups depicted in the compounds in Table 1, below.

As defined generally above, A² is phenylene, C₃₋₆ cycloalkyl, or pyridinylene, each of which is substituted with 0 or 1 occurrence of R⁷. In certain embodiments, A² is phenylene or pyridinylene, each of which is substituted with 0 or 1 occurrence of R⁷. In certain embodiments, A² is phenylene substituted with 0 or 1 occurrence of R⁷. In certain embodiments, A² is pyridinylene substituted with 0 or 1 occurrence of R⁷. In certain embodiments, A² is phenylene substituted with 1 occurrence of R⁷. In certain embodiments, A² is pyridinylene substituted with 1 occurrence of R⁷. In certain embodiments, A² is phenylene. In certain embodiments, A² is C₃₋₆ cycloalkyl substituted with 1 occurrence of R⁷. In certain embodiments, A² is C₃₋₆ cycloalkyl. In certain embodiments, A² is pyridinylene. In certain embodiments, A² is selected from the groups depicted in the compounds in Table 1, below.

As defined generally above, A³ is a 5-membered heteroaryl or heterocyclyl containing 2, 3, or 4 heteroatoms selected from nitrogen and sulphur, wherein the heteroaryl or heterocyclyl is substituted with n occurrences of R⁸. In certain embodiments, A³ is a 5-membered heteroaryl or heterocyclyl containing 2, 3, or 4 heteroatoms selected from nitrogen and sulphur, wherein the heteroaryl is substituted with n occurrences of R⁸. In certain embodiments, A³ is a 5-membered heteroaryl or heterocyclyl containing 2 or 3 heteroatoms selected from nitrogen and sulphur, wherein the heteroaryl is substituted with n occurrences of R⁸. In certain embodiments, A³ is a 5-membered heteroaryl or heterocyclyl containing 4 heteroatoms selected from nitrogen and sulphur, wherein the heteroaryl is substituted with n occurrences of R⁸. In certain embodiments, the heteroatoms are selected from nitrogen.

In certain embodiments, A³ is a 5-membered heteroaryl or heterocyclyl containing 2, 3, or 4 heteroatoms selected from nitrogen and sulphur, wherein the heteroaryl or heterocyclyl is substituted with n occurrences of R⁸. In certain embodiments, A³ is a 5-membered heteroaryl containing 2, 3, or 4 heteroatoms selected from nitrogen, wherein the heteroaryl is substituted with n occurrences of R⁸. In certain embodiments. A³ is a 5-membered heteroaryl containing 2 or 3 heteroatoms selected from nitrogen, wherein the heteroaryl is substituted with n occurrences of R⁸. In certain embodiments, A³ is a 5-membered heteroaryl containing 4 heteroatoms selected from nitrogen, wherein the heteroaryl is substituted with n occurrences of R⁸. In certain embodiments. A³ is pyrazolyl substituted with n occurrences of R⁸. In certain embodiments. A³ is pyrazolyl.

In certain embodiments, A³ is

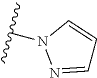 [SMILES: *n1cccn1]

substituted with n occurrences of R⁸. In certain embodiments, A³ is

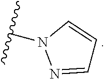 [SMILES: *n1cccn1]

In certain embodiments, A³ is one of the following:

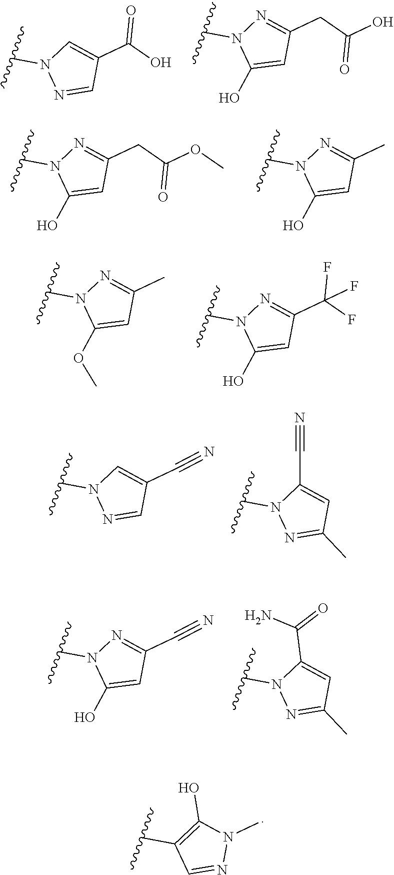

In certain embodiments, wherein A³ is tetrazolyl. In certain embodiments, A³ is

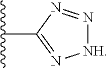 [SMILES: *c1nn[nH]n1]

In certain embodiments, A³ is selected from the groups depicted in the compounds in Table 1, below.

As defined generally above, X¹ is —(C₀₋₃ alkylene)-N(R⁹)C(O)-Ψ, —(C₀₋₃ alkylene)-N(R⁹)-Ψ, -(3-6 membered saturated heterocyclylene containing 1 or 2 heteroatoms selected from nitrogen)-(C₀₋₃ alkylene)-Ψ, or -(3-5 membered saturated heterocyclylene containing 1 or 2 heteroatoms selected from nitrogen)-C(O)-Ψ; wherein Ψ is a bond to A². In certain embodiments, X¹ is —(C₀₋₃ alkylene)-N(R⁹)C(O)-Ψ or -(3-5 membered saturated heterocyclylene containing 1 or 2 heteroatoms selected from nitrogen)-C(O)-Ψ; wherein Ψ is a bond to A². In certain embodiments, X¹ is —(C₀₋₃ alkylene)-N(R⁹)C(O)-Ψ. In certain embodiments, X¹ is —(C₁₋₃ alkylene)-N(R⁹)C(O)-Ψ, wherein Ψ is a bond to A². In certain embodiments, X¹ is —(C₀₋₃ alkylene)-N(R⁹)-Ψ. In certain embodiments, X¹ is -(3-6 membered saturated heterocyclylene containing 1 or 2 heteroatoms selected from nitrogen)-(C₀₋₃ alkylene)-Ψ. In certain embodiments, X¹ is —N(R⁹)C(O)-Ψ. In certain embodiments, X¹ is -(3-5 membered saturated heterocyclylene containing 1 or 2 heteroatoms selected from nitrogen)-C(O)-Ψ; wherein Ψ is a bond to A². In certain embodiments, X¹ is —(C₁₋₃ alkylene)-N(R⁹)C(O)-Ψ. In certain embodiments, X¹ is —(CH₂)—N(R⁹)C(O)-Ψ. In certain embodiments, X¹ is a -(3-5 membered saturated heterocyclylene containing 1 or 2 heteroatoms selected from nitrogen)-C(O)-Ψ. In certain embodiments, X¹ is a -(4-membered saturated heterocyclylene containing 1 heteroatom selected from nitrogen)-C(O)-Ψ. In certain embodiments, X¹ is

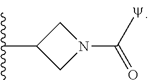 [SMILES: *C1CN(C(C)=O)C1]

In certain embodiments, X¹ is selected from the groups depicted in the compounds in Table 1, below.

As defined generally above, Y¹ is N or —C(H)—. In certain embodiments, Y¹ is N. In certain embodiments, Y¹ is —C(H)—. In certain embodiments, Y¹ is selected from the groups depicted in the compounds in Table 1, below.

As defined generally above, n is 0, 1, or 2. In certain embodiments, n is 1. In certain embodiments, n is 2. In certain embodiments, n is a value according to that depicted in the compounds in Table 1, below.

The description above describes multiple embodiments relating to compounds of Formula I. The patent application specifically contemplates all combinations of the embodiments.

In certain embodiments, the compound is represented by Formula I-aa, I-ab, I-ac. I-ad, or I-ae, or a pharmaceutically acceptable salt thereof:

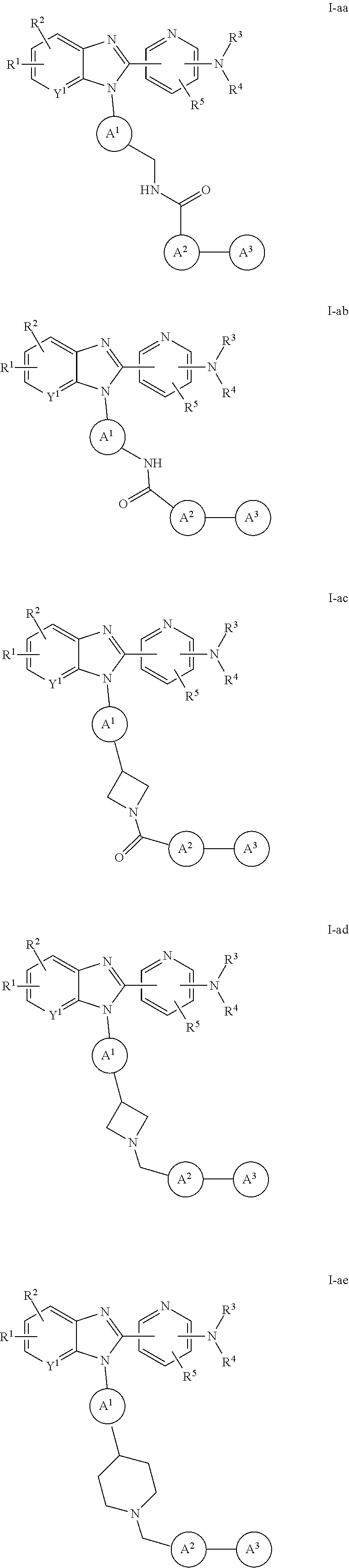

In certain embodiments, the compound is represented by Formula I-aa, I-ab, I-ad, or I-ae, or a pharmaceutically acceptable salt thereof:

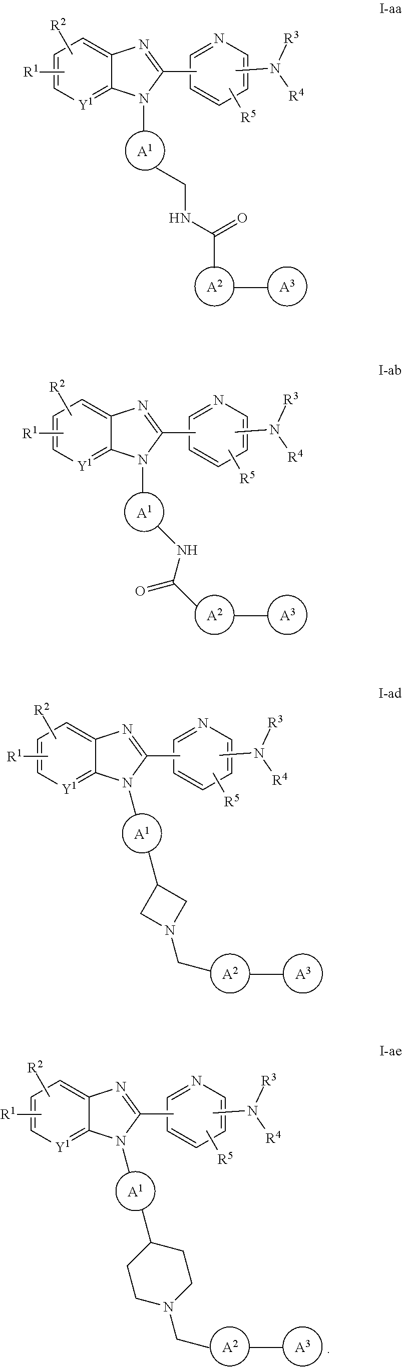

In certain embodiments, the compound is represented by Formula Ia or a pharmaceutically acceptable salt thereof:

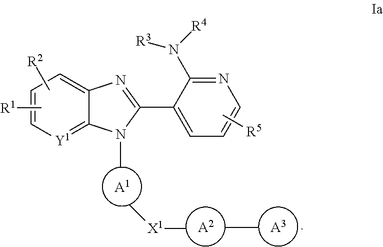

In certain embodiments, the compound is a compound of Formula Ib, Ic, Id, Ie, If, or Ig, or a pharmaceutically acceptable salt thereof:

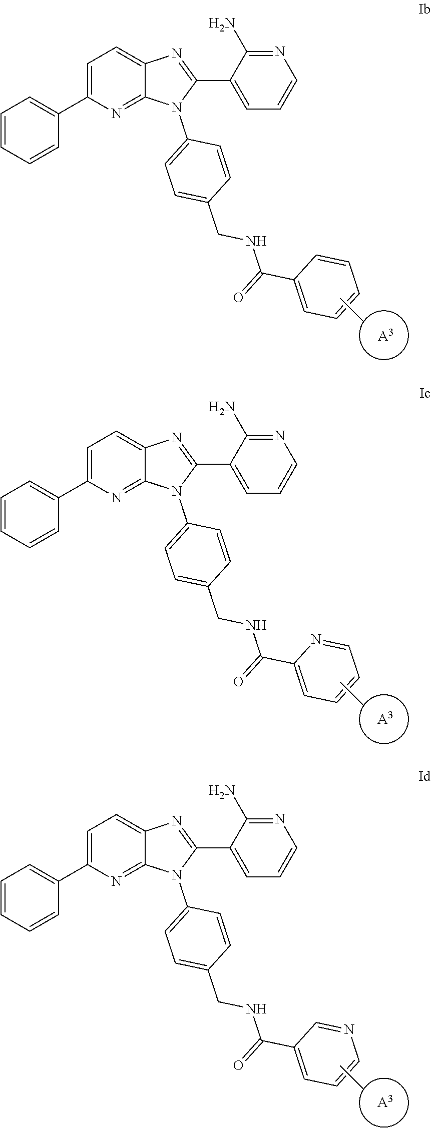

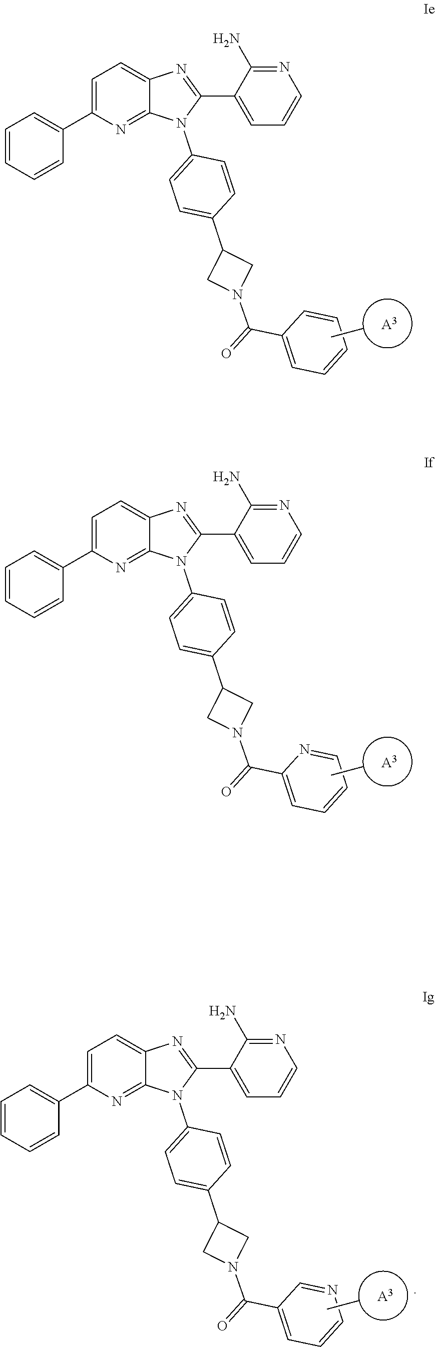

In certain embodiments, the compound is a compound of Formula Ih, I, Ij, Ik, Il, or Im, or a pharmaceutically acceptable salt thereof:

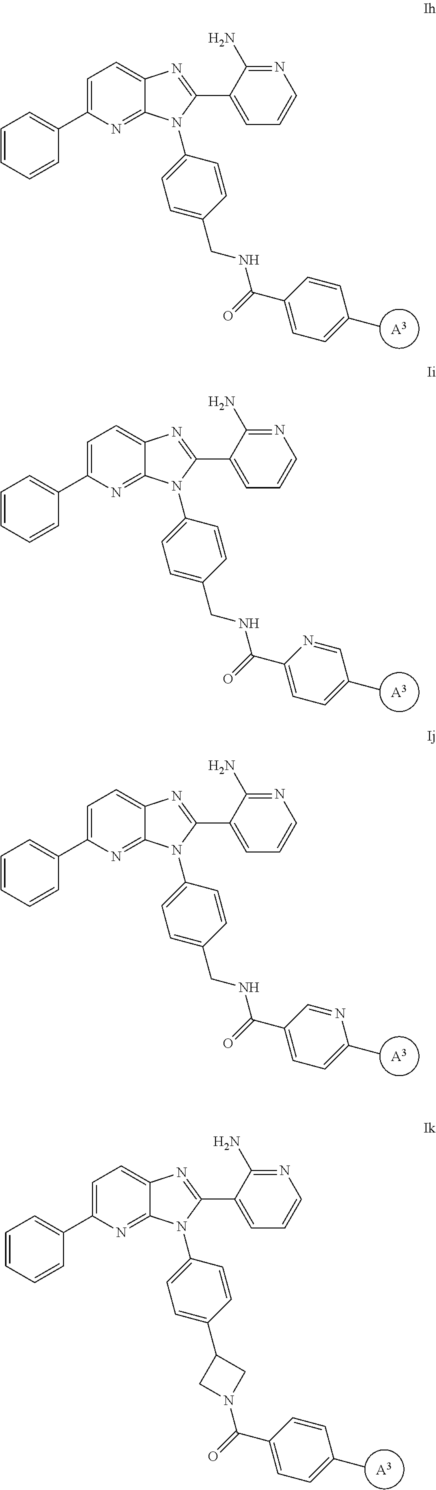

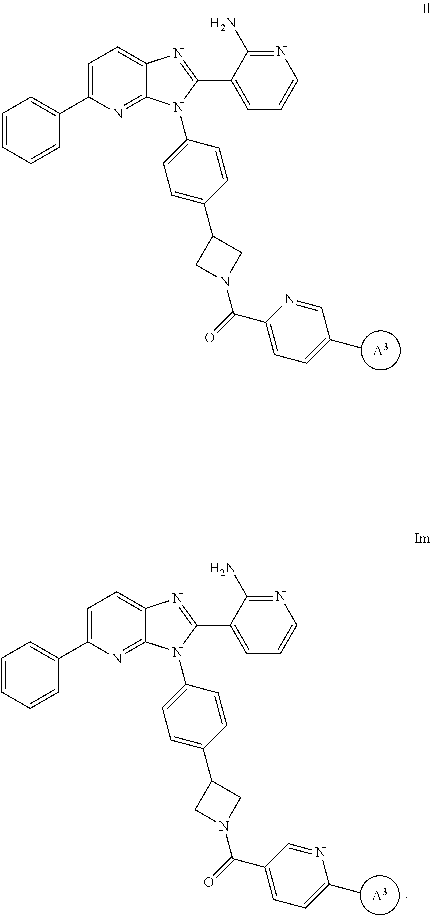

### Exemplary Specific Compounds

In certain embodiments, the compound is a compound in Table 1 or a stereoisomer or pharmaceutically acceptable salt thereof. In certain embodiments, the compound is a compound in Table 1. It is contemplated herein, in certain embodiments, that any free carboxylic acid present in a compound in Table 1 could be readily replaced with a corresponding ester group (e.g, a methyl ester). It is also contemplated herein, in certain embodiments, that any ester group (e.g, a methyl ester) present in a compound in Table 1 could be readily replaced with a free carboxylic acid.

TABLE 1

| Compound No. | Chemical Structure |
|---|---|
| I-1 | 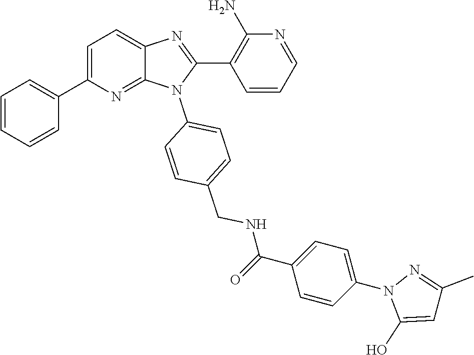 [SMILES: Cc1cc(O)n(-c2ccc(C(=O)NCc3ccc(-n4c(-c5cccnc5N)nc5ccc(-c6ccccc6)nc54)cc3)cc2)n1] |
| I-2 | 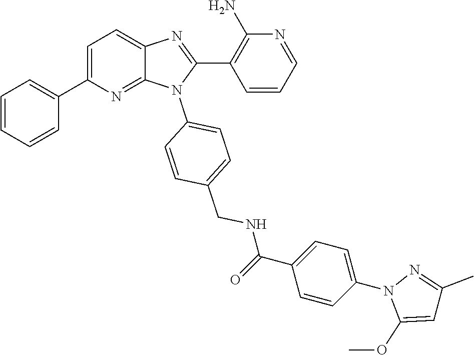 [SMILES: COc1cc(C)nn1-c1ccc(C(=O)NCc2ccc(-n3c(-c4cccnc4N)nc4ccc(-c5ccccc5)nc43)cc2)cc1] |
| I-3 | 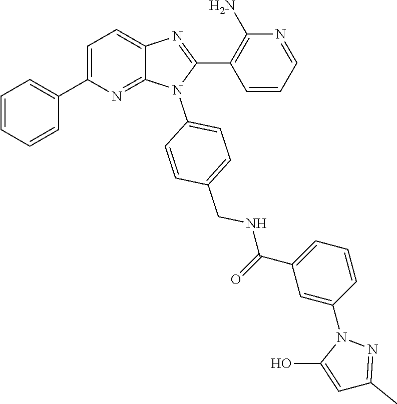 [SMILES: Cc1cc(O)n(-c2cccc(C(=O)NCc3ccc(-n4c(-c5cccnc5N)nc5ccc(-c6ccccc6)nc54)cc3)c2)n1] |
| I-4 | 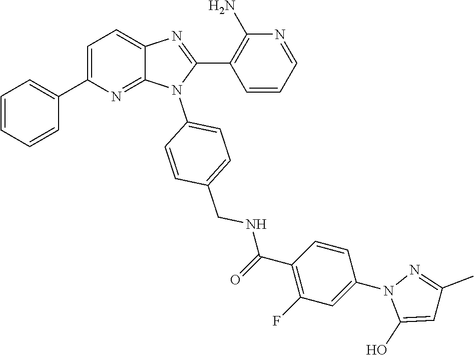 [SMILES: Cc1cc(O)n(-c2ccc(C(=O)NCc3ccc(-n4c(-c5cccnc5N)nc5ccc(-c6ccccc6)nc54)cc3)c(F)c2)n1] |
| I-5 | 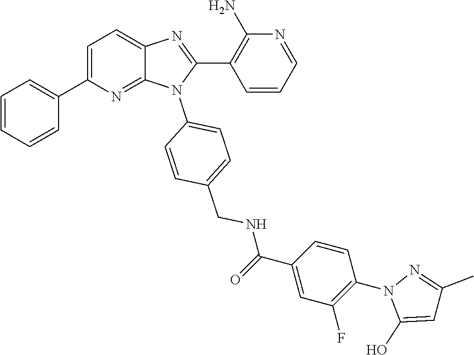 [SMILES: Cc1cc(O)n(-c2ccc(C(=O)NCc3ccc(-n4c(-c5cccnc5N)nc5ccc(-c6ccccc6)nc54)cc3)cc2F)n1] |
| I-6 | 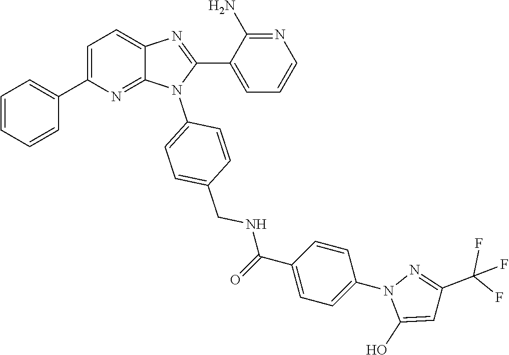 [SMILES: Nc1ncccc1-c1nc2ccc(-c3ccccc3)nc2n1-c1ccc(CNC(=O)c2ccc(-n3nc(C(F)(F)F)cc3O)cc2)cc1] |
| I-7 | 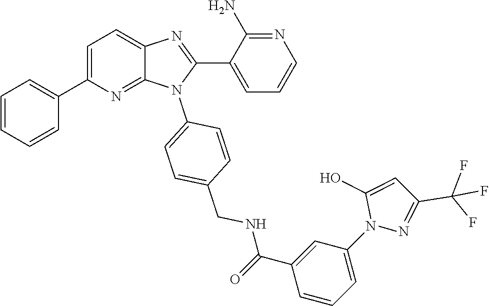 [SMILES: Nc1ncccc1-c1nc2ccc(-c3ccccc3)nc2n1-c1ccc(CNC(=O)c2cccc(-n3nc(C(F)(F)F)cc3O)c2)cc1] |
| I-8 | 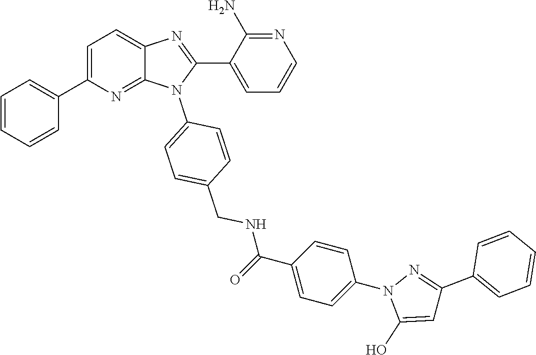 [SMILES: Nc1ncccc1-c1nc2ccc(-c3ccccc3)nc2n1-c1ccc(CNC(=O)c2ccc(-n3nc(-c4ccccc4)cc3O)cc2)cc1] |
| I-9 | 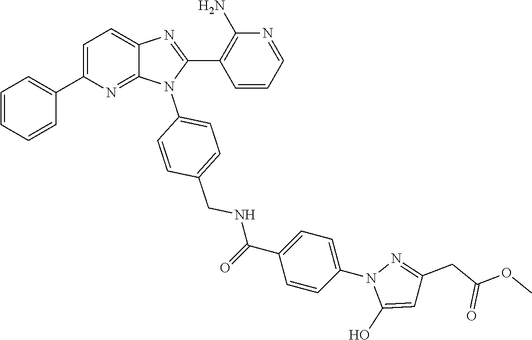 [SMILES: COC(=O)Cc1cc(O)n(-c2ccc(C(=O)NCc3ccc(-n4c(-c5cccnc5N)nc5ccc(-c6ccccc6)nc54)cc3)cc2)n1] |
| I-10 | 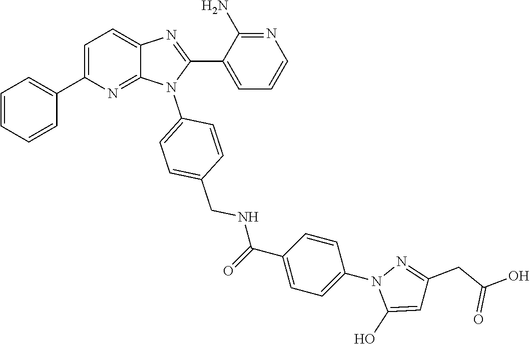 [SMILES: Nc1ncccc1-c1nc2ccc(-c3ccccc3)nc2n1-c1ccc(CNC(=O)c2ccc(-n3nc(CC(=O)O)cc3O)cc2)cc1] |
| I-11 | 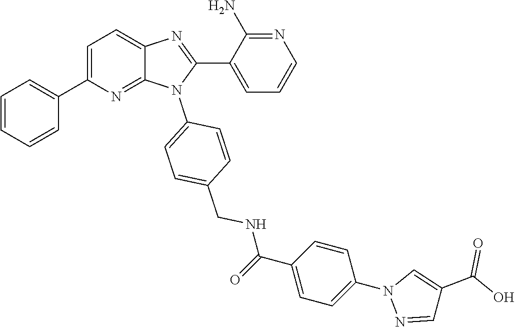 [SMILES: Nc1ncccc1-c1nc2ccc(-c3ccccc3)nc2n1-c1ccc(CNC(=O)c2ccc(-n3cc(C(=O)O)cn3)cc2)cc1] |
| I-12 | 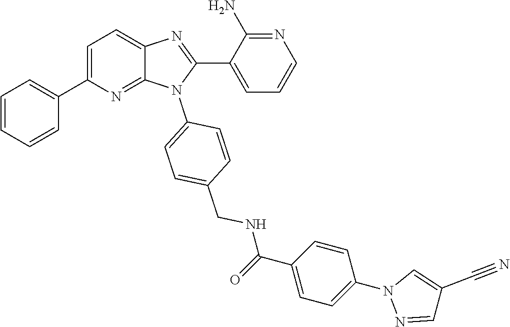 [SMILES: N#Cc1cnn(-c2ccc(C(=O)NCc3ccc(-n4c(-c5cccnc5N)nc5ccc(-c6ccccc6)nc54)cc3)cc2)c1] |
| I-13 | 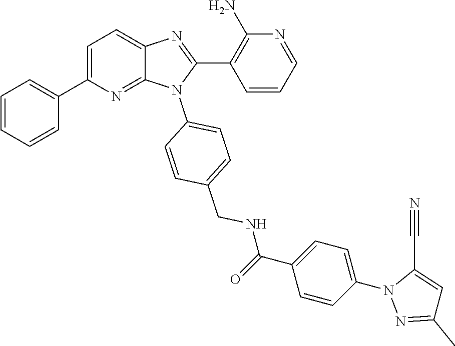 [SMILES: Cc1cc(C#N)n(-c2ccc(C(=O)NCc3ccc(-n4c(-c5cccnc5N)nc5ccc(-c6ccccc6)nc54)cc3)cc2)n1] |
| I-14 | 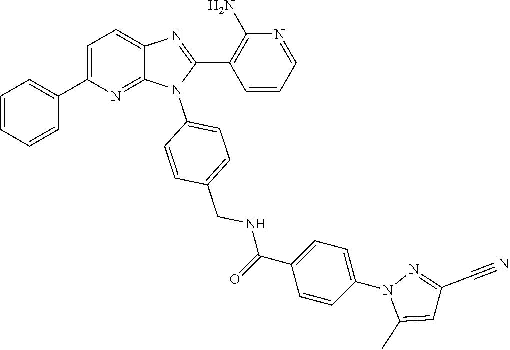 [SMILES: Cc1cc(C#N)nn1-c1ccc(C(=O)NCc2ccc(-n3c(-c4cccnc4N)nc4ccc(-c5ccccc5)nc43)cc2)cc1] |
| I-15 | 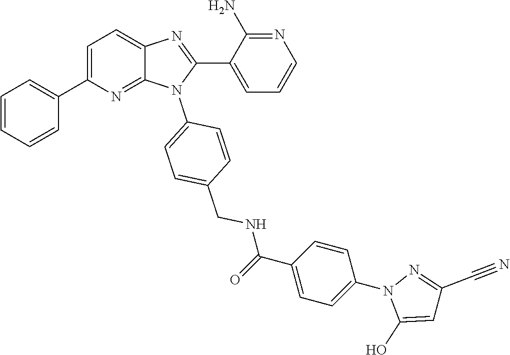 [SMILES: N#Cc1cc(O)n(-c2ccc(C(=O)NCc3ccc(-n4c(-c5cccnc5N)nc5ccc(-c6ccccc6)nc54)cc3)cc2)n1] |
| I-16 | 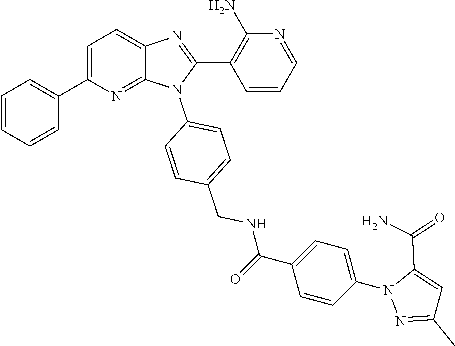 [SMILES: Cc1cc(C(N)=O)n(-c2ccc(C(=O)NCc3ccc(-n4c(-c5cccnc5N)nc5ccc(-c6ccccc6)nc54)cc3)cc2)n1] |
| I-17 | 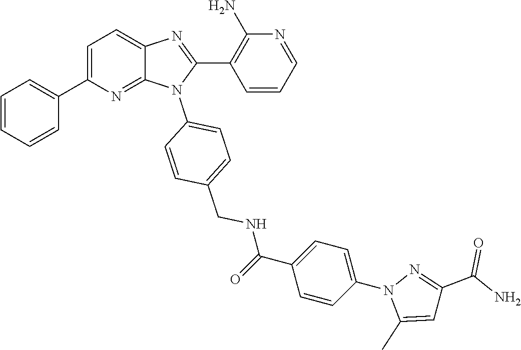 [SMILES: Cc1cc(C(N)=O)nn1-c1ccc(C(=O)NCc2ccc(-n3c(-c4cccnc4N)nc4ccc(-c5ccccc5)nc43)cc2)cc1] |
| I-18 | 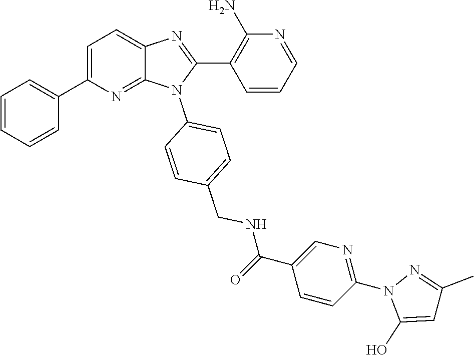 [SMILES: Cc1cc(O)n(-c2ccc(C(=O)NCc3ccc(-n4c(-c5cccnc5N)nc5ccc(-c6ccccc6)nc54)cc3)cn2)n1] |
| I-19 | 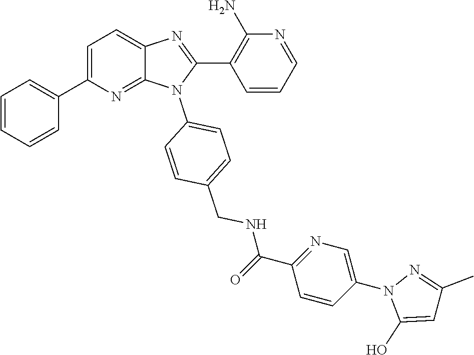 [SMILES: Cc1cc(O)n(-c2ccc(C(=O)NCc3ccc(-n4c(-c5cccnc5N)nc5ccc(-c6ccccc6)nc54)cc3)nc2)n1] |
| I-20 | 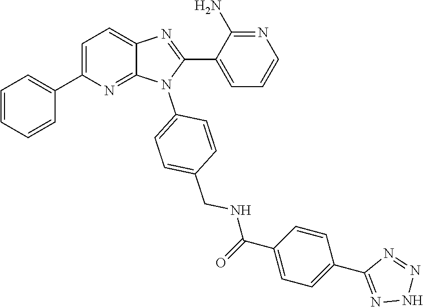 [SMILES: Nc1ncccc1-c1nc2ccc(-c3ccccc3)nc2n1-c1ccc(CNC(=O)c2ccc(-c3nn[nH]n3)cc2)cc1] |
| I-21 | 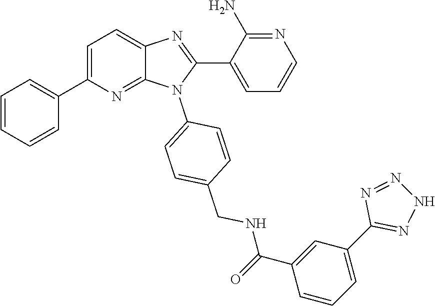 [SMILES: Nc1ncccc1-c1nc2ccc(-c3ccccc3)nc2n1-c1ccc(CNC(=O)c2cccc(-c3nn[nH]n3)c2)cc1] |
| I-22 | 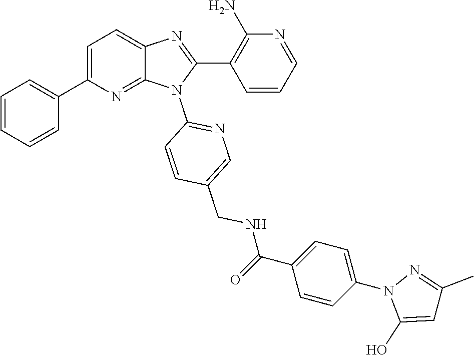 [SMILES: Cc1cc(O)n(-c2ccc(C(=O)NCc3ccc(-n4c(-c5cccnc5N)nc5ccc(-c6ccccc6)nc54)nc3)cc2)n1] |
| I-23 | 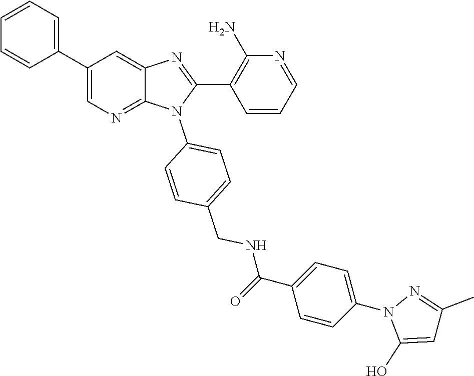 [SMILES: Cc1cc(O)n(-c2ccc(C(=O)NCc3ccc(-n4c(-c5cccnc5N)nc5cc(-c6ccccc6)cnc54)cc3)cc2)n1] |
| I-24 | 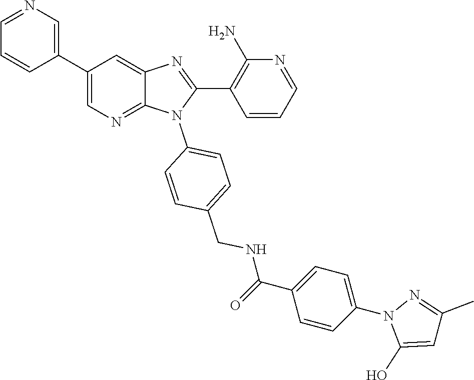 [SMILES: Cc1cc(O)n(-c2ccc(C(=O)NCc3ccc(-n4c(-c5cccnc5N)nc5cc(-c6cccnc6)cnc54)cc3)cc2)n1] |
| I-25 | 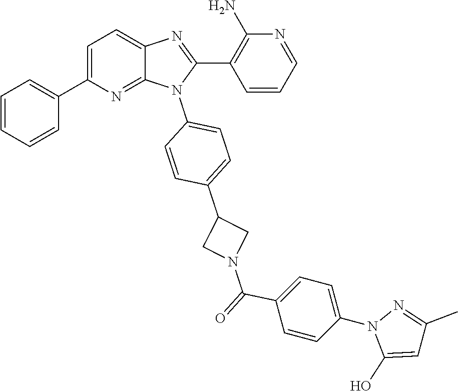 [SMILES: Cc1cc(O)n(-c2ccc(C(=O)N3CC(c4ccc(-n5c(-c6cccnc6N)nc6ccc(-c7ccccc7)nc65)cc4)C3)cc2)n1] |
| I-26 | 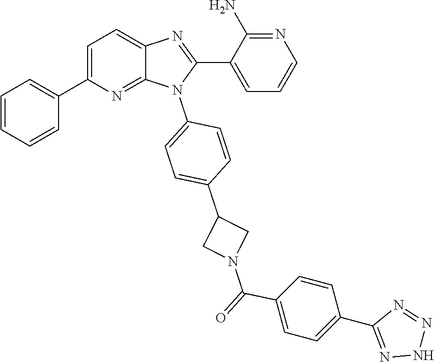 [SMILES: Nc1ncccc1-c1nc2ccc(-c3ccccc3)nc2n1-c1ccc(C2CN(C(=O)c3ccc(-c4nn[nH]n4)cc3)C2)cc1] |
| I-27 | 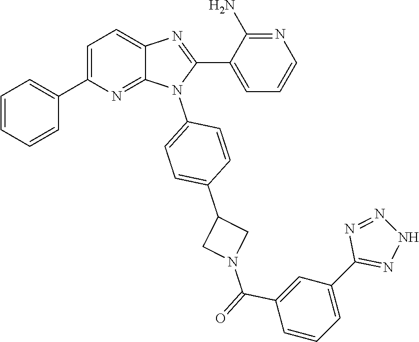 [SMILES: Nc1ncccc1-c1nc2ccc(-c3ccccc3)nc2n1-c1ccc(C2CN(C(=O)c3cccc(-c4nn[nH]n4)c3)C2)cc1] |
| I-28 | 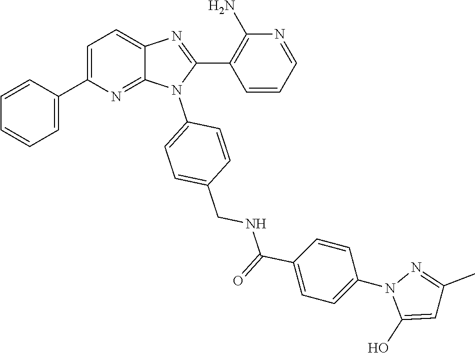 [SMILES: Cc1cc(O)n(-c2ccc(C(=O)NCc3ccc(-n4c(-c5cccnc5N)nc5ccc(-c6ccccc6)nc54)cc3)cc2)n1] |
| I-29 | 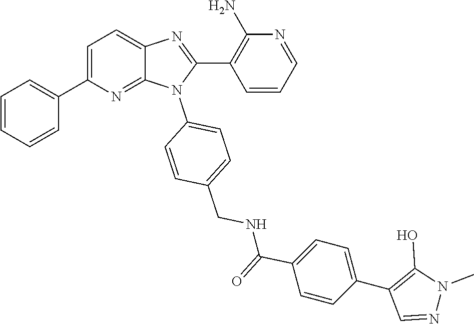 [SMILES: Cn1ncc(-c2ccc(C(=O)NCc3ccc(-n4c(-c5cccnc5N)nc5ccc(-c6ccccc6)nc54)cc3)cc2)c1O] |
| I-30 | 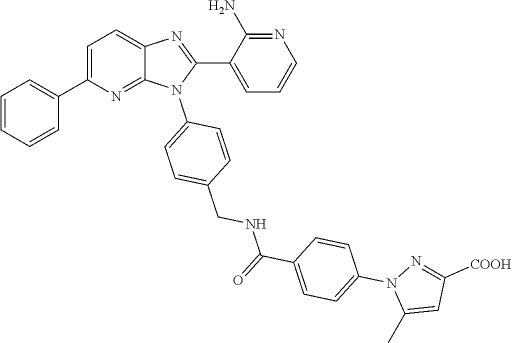 [SMILES: Cc1cc(C(=O)O)nn1-c1ccc(C(=O)NCc2ccc(-n3c(-c4cccnc4N)nc4ccc(-c5ccccc5)nc43)cc2)cc1] |
| I-31 | 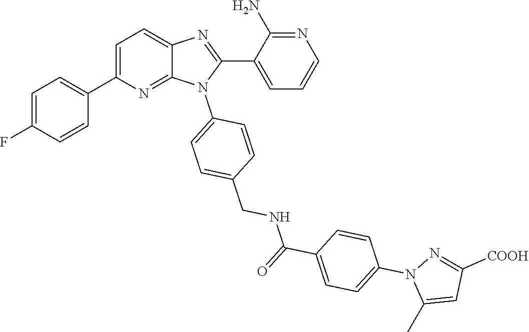 [SMILES: Cc1cc(C(=O)O)nn1-c1ccc(C(=O)NCc2ccc(-n3c(-c4cccnc4N)nc4ccc(-c5ccc(F)cc5)nc43)cc2)cc1] |
| I-32 | 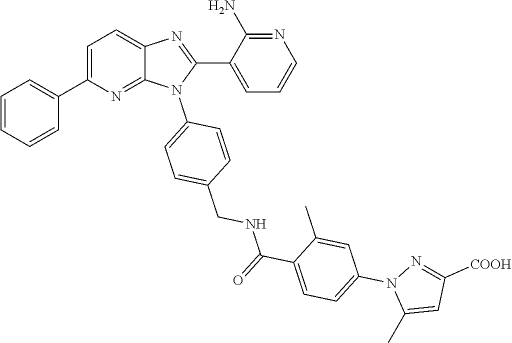 [SMILES: Cc1cc(-n2nc(C(=O)O)cc2C)ccc1C(=O)NCc1ccc(-n2c(-c3cccnc3N)nc3ccc(-c4ccccc4)nc32)cc1] |
| I-33 | 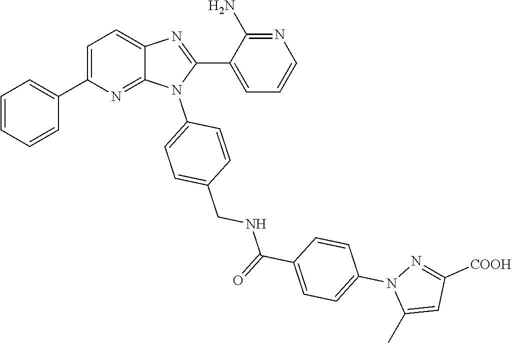 [SMILES: Cc1cc(C(=O)O)nn1-c1ccc(C(=O)NCc2ccc(-n3c(-c4cccnc4N)nc4ccc(-c5ccccc5)nc43)cc2)cc1] |
| I-34 | 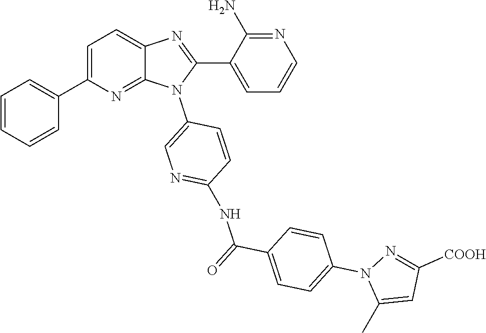 [SMILES: Cc1cc(C(=O)O)nn1-c1ccc(C(=O)Nc2ccc(-n3c(-c4cccnc4N)nc4ccc(-c5ccccc5)nc43)cn2)cc1] |
| I-35 | 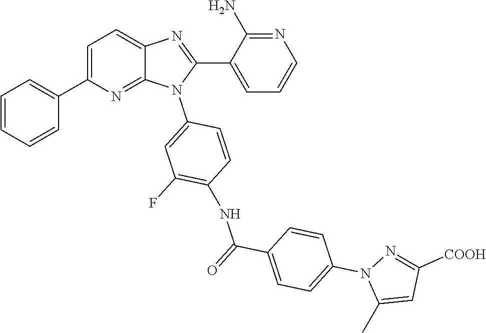 [SMILES: Cc1cc(C(=O)O)nn1-c1ccc(C(=O)Nc2ccc(-n3c(-c4cccnc4N)nc4ccc(-c5ccccc5)nc43)cc2F)cc1] |
| I-36 | 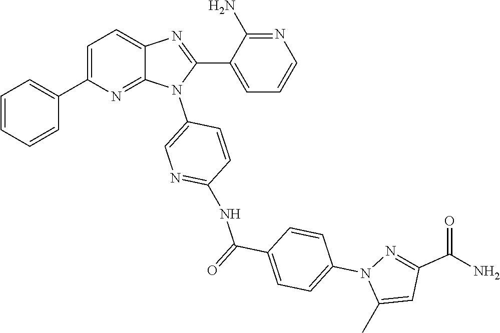 [SMILES: Cc1cc(C(N)=O)nn1-c1ccc(C(=O)Nc2ccc(-n3c(-c4cccnc4N)nc4ccc(-c5ccccc5)nc43)cn2)cc1] |
| I-37 | 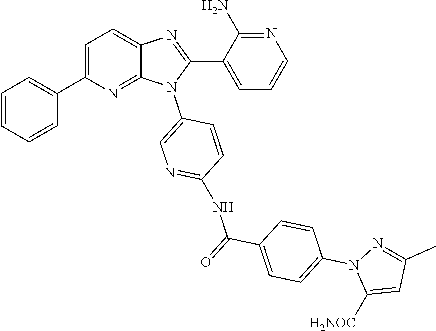 [SMILES: Cc1cc(C(N)=O)n(-c2ccc(C(=O)Nc3ccc(-n4c(-c5cccnc5N)nc5ccc(-c6ccccc6)nc54)cn3)cc2)n1] |
| I-38 | 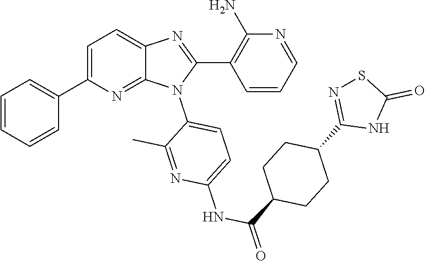 [SMILES: Cc1nc(NC(=O)[C@H]2CC[C@H](c3nsc(=O)[nH]3)CC2)ccc1-n1c(-c2cccnc2N)nc2ccc(-c3ccccc3)nc21] |
| I-39 | 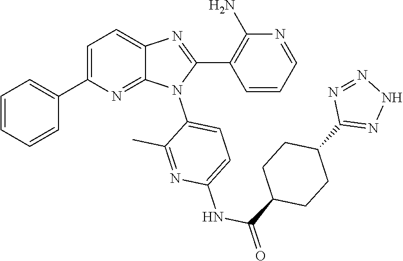 [SMILES: Cc1nc(NC(=O)[C@H]2CC[C@H](c3nn[nH]n3)CC2)ccc1-n1c(-c2cccnc2N)nc2ccc(-c3ccccc3)nc21] |
| I-40 | 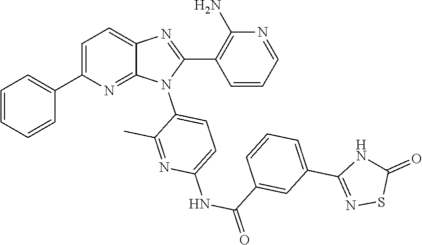 [SMILES: Cc1nc(NC(=O)c2cccc(-c3nsc(=O)[nH]3)c2)ccc1-n1c(-c2cccnc2N)nc2ccc(-c3ccccc3)nc21] |
| I-41 | 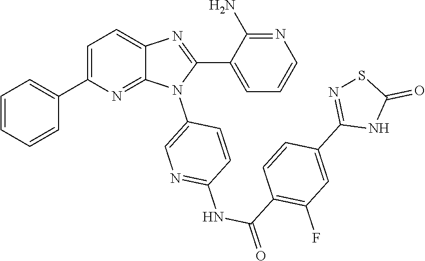 [SMILES: Nc1ncccc1-c1nc2ccc(-c3ccccc3)nc2n1-c1ccc(NC(=O)c2ccc(-c3nsc(=O)[nH]3)cc2F)nc1] |
| I-42 | 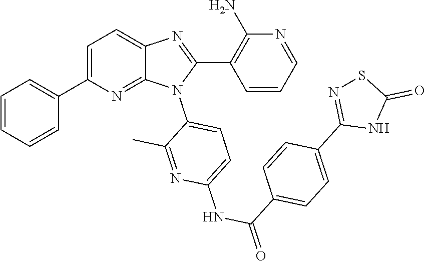 [SMILES: Cc1nc(NC(=O)c2ccc(-c3nsc(=O)[nH]3)cc2)ccc1-n1c(-c2cccnc2N)nc2ccc(-c3ccccc3)nc21] |
| I-43 | 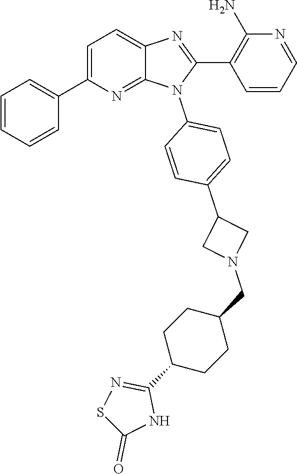 [SMILES: Nc1ncccc1-c1nc2ccc(-c3ccccc3)nc2n1-c1ccc(C2CN(C[C@H]3CC[C@H](c4nsc(=O)[nH]4)CC3)C2)cc1] |
| I-44 | 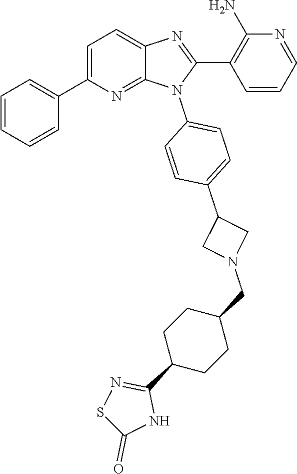 [SMILES: Nc1ncccc1-c1nc2ccc(-c3ccccc3)nc2n1-c1ccc(C2CN(C[C@H]3CC[C@@H](c4nsc(=O)[nH]4)CC3)C2)cc1] |
| I-45 | 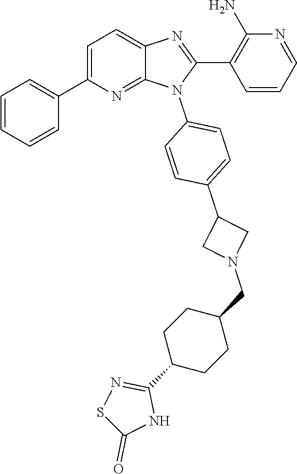 [SMILES: Nc1ncccc1-c1nc2ccc(-c3ccccc3)nc2n1-c1ccc(C2CN(C[C@H]3CC[C@H](c4nsc(=O)[nH]4)CC3)C2)cc1] |
| I-46 | 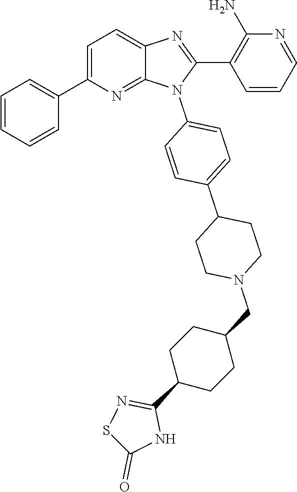 [SMILES: Nc1ncccc1-c1nc2ccc(-c3ccccc3)nc2n1-c1ccc(C2CCN(C[C@H]3CC[C@@H](c4nsc(=O)[nH]4)CC3)CC2)cc1] |
| I-47 | 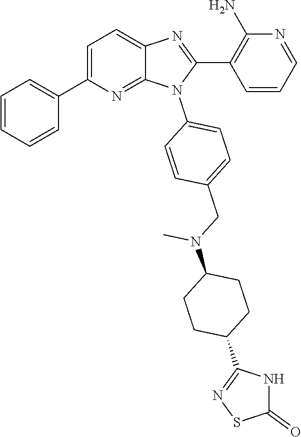 [SMILES: CN(Cc1ccc(-n2c(-c3cccnc3N)nc3ccc(-c4ccccc4)nc32)cc1)[C@H]1CC[C@H](c2nsc(=O)[nH]2)CC1] |
| I-48 | 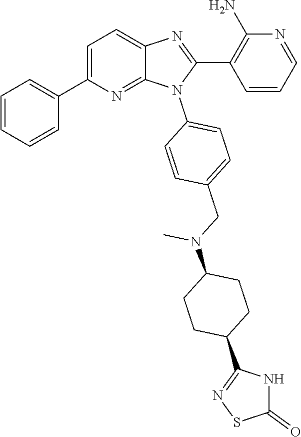 [SMILES: CN(Cc1ccc(-n2c(-c3cccnc3N)nc3ccc(-c4ccccc4)nc32)cc1)[C@H]1CC[C@@H](c2nsc(=O)[nH]2)CC1] |

### Synthetic Methods

Methods for preparing compounds described herein are illustrated in the following synthetic schemes (Scheme 1-Scheme 4). The schemes are given for the purpose of illustrating the invention, and not intended to limit the scope or spirit of the invention. Starting materials shown in the schemes can be obtained from commercial sources or can be prepared based on procedures described in the literature.

Scheme 1 illustrates a general method for aminopyridinyl imidazo[4,5-b]pyridine compounds F. Metal-mediated (e.g., using Cu(OAc)₂) coupling of pyrazole A with boronic acid B provides pyrazolyl-methyl benzoate C. Treatment of pyrazolyl-methyl benzoate C with base provides carboxylic acid D. Amide coupling (e.g., using HATU) of carboxylic acid D with amine E provides the final aminopyridinyl imidazo[4,5-b]pyridine compound F, after optional hydrolysis.

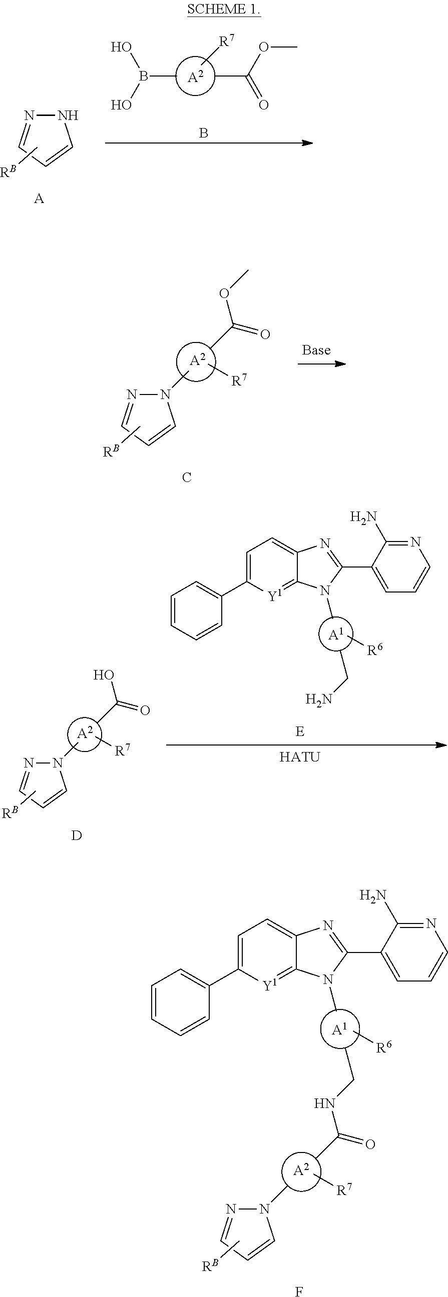

Scheme 2 illustrates a general method for preparing aminopyridinyl imidazo[4,5-b]pyridine compounds. Reaction of A with B provides C. Reaction of C with aldehyde D, and Boc deprotection provides imidazo[4,5-b]pyridine E, which is then converted to final aminopyridinyl imidazo[4,5-b]pyridine compound F, after optional hydrolysis.

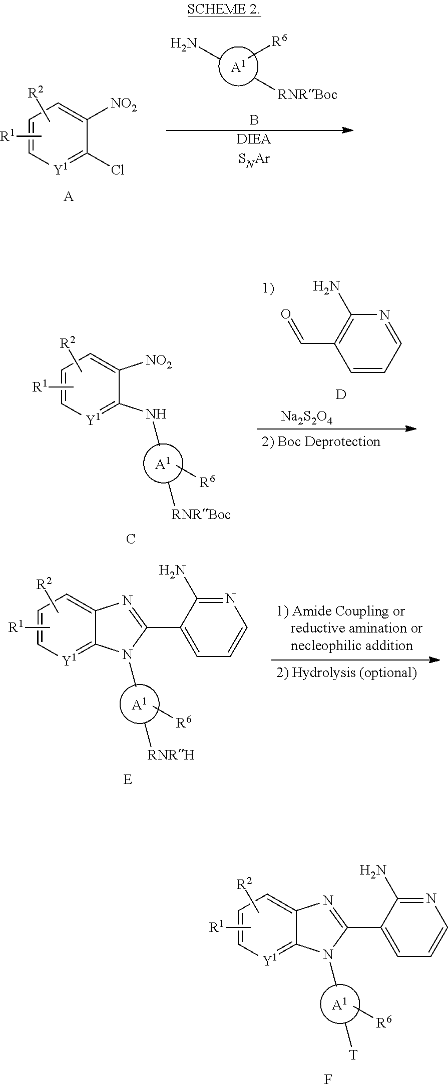

Scheme 3 illustrates a general method for preparing aminopyridinyl imidazo[4,5-b]pyridine compounds. Reaction of A with B provides C, which is then converted to D. Reaction of D with aldehyde E provides the final imidazo[4,5-b]pyridine compound F, after optional hydrolysis.

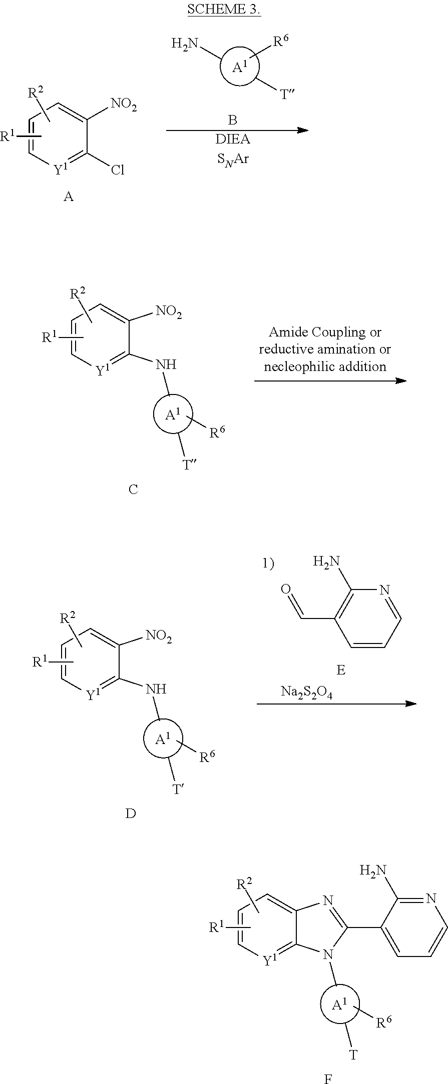

Scheme 4 illustrates a general method for preparing aminopyridinyl imidazo[4,5-b]pyridine compounds. Reaction of A with B provides C. Nitro C is reduced to provide amine D. Reaction of D with aldehyde E provides the final imidazo[4,5-b]pyridine compound F, after optional hydrolysis.

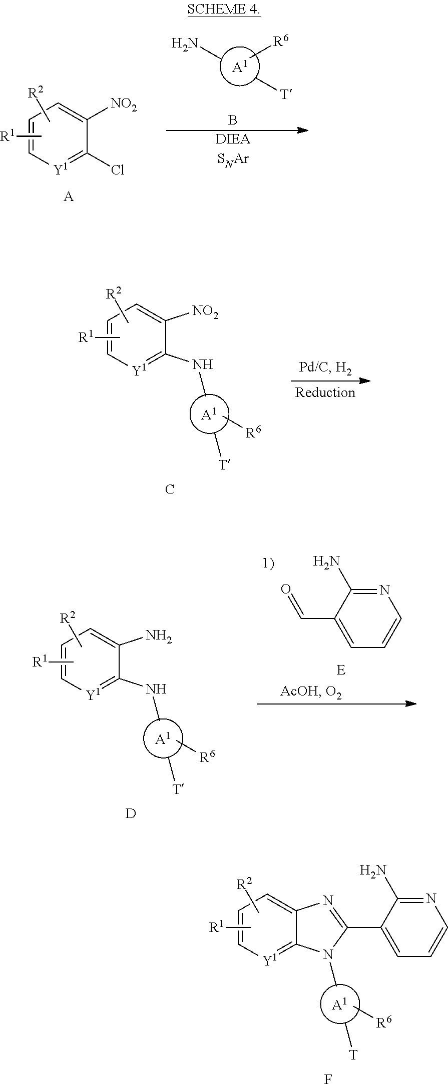

In the schemes (Scheme 1-Scheme 4) described above, T is

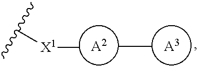

wherein “ [symbol] ” represents a bond to A¹, T′ and T″ refer to earlier stages in synthesis of T and Y¹, R¹, R², R⁶, R⁷, R⁸, A¹, X¹, A², and A³ are as defined herein. In the schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference. The modular synthetic route illustrated in the schemes can also be readily modified by one of skill in the art to provide additional compounds by conducting functional group transformations on the intermediate and final compounds. Such functional group transformations are well known in the art, as described in, for example. “Comprehensive Organic Synthesis” (B. M. Trost & I. Fleming, eds., 1991-1992).

### II. Therapeutic Applications

Another aspect of the invention provides a method of treating a disease or disorder associated with aberrant AKT1 signaling, wherein the method comprises administering to a subject in need thereof a therapeutically effective amount of a compound described herein, such as a compound of Formula I, to treat the disease or disorder associated with aberrant AKT1 signaling. In certain embodiments, the particular compound of Formula I is a compound defined by one of the embodiments described in Section I, above. In certain embodiments, the disease or disorder associated with aberrant AKT1 signaling is an AKT1 E17K associated disease or disorder.

Another aspect of the invention provides a method of treating cancer, wherein the method comprises administering to a subject in need thereof a therapeutically effective amount of a compound described herein, such as a compound of Formula I, to treat the cancer. In certain embodiments, the particular compound of Formula I is a compound defined by one of the embodiments described in Section I, above.

Methods described herein may be further defined according to additional features, such as the identity of the cancer and/or the subject.

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

In certain embodiments, the cancer is a solid tumor.

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, uterine corpus endometrial carcinoma, cervical cancer, endocervical cancer, thyroid carcinoma, prostate adenocarcinoma, skin cutaneous melanoma, bladder urothelial carcinoma, head and neck squamous cell carcinoma, or stomach adenocarcinoma.

In certain embodiments, the cancer is an adenocarcinoma, squamous cell carcinoma, epithelial neoplasm, glioma, ductal neoplasm, lobular neoplasm, cystic neoplasm, mucinous neoplasm, or serous neoplasm, acinar cell neoplasm, basal cell neoplasm, fibroepithelial neoplasm, transitional cell papilloma, or transitional cell carcinoma.

In certain embodiments, the cancer is a cervical cancer, uterine cancer, breast cancer, thyroid cancer, prostate cancer, lung cancer, bladder cancer, skin cancer, stomach cancer, lymphoma, or leukemia.

In certain embodiments, the cancer is a lymphoma or leukemia.

In certain embodiments, the cancer has active PI3K signaling. In certain embodiments, the cancer has one or more mutations in phosphatase and tensin homolog (PTEN), phosphatidylinositol-4,5-bisphosphate 3-kinase catalytic subunit alpha (PIK3CA), phosphatidylinositol-4,5-bisphosphate 3-kinase catalytic subunit beta (PIK3CB), phosphatidylinositol-4,5-bisphosphate 3-kinase catalytic subunit delta (PIK3CD), mechanistic target of rapamycin kinase (mTOR), RPTOR independent companion of mTOR complex 2 (RICTOR), MAPK associated protein 1 (MAPKAP1), or 3-phosphoinositide dependent protein kinase 1 (PDPK1), or combinations thereof.

In certain embodiments, the cancer has an AKT1 mutation. In certain embodiments, the cancer has an AKT1 E17K mutation.

Exemplary cancers reported in the literature having an AKT1 E17K mutation include breast invasive carcinoma, uterine corpus endometrial carcinoma, colon adenocarcinoma, cervical and endocervical cancer, thyroid carcinoma, lung adenocarcinoma, prostate adenocarcinoma, skin cutaneous melanoma, bladder urothelial carcinoma, head and neck squamous cell carcinoma, and stomach adenocarcinoma.

Additionally, the AKT1E17K mutation has been in reported in many sub-types of breast cancer including ductal, lobular, and in many combinations of HER2, estrogen receptor and progesterone receptor positivity. See, for example, BMC Cancer volume 16, Article number: 622 (2016)).

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, or metastatic melanoma. In certain embodiments, the cancer is a melanoma.

In certain embodiments, the disorder is a cancer selected from the group consisting of ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, and leukemia.

In certain embodiments, the cancer is a solid tumor. In certain embodiments, the cancer is a sarcoma or carcinoma. In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is prostate cancer, breast cancer, lung cancer, liver cancer, bladder cancer, urinary tract cancer, or eye cancer. In certain embodiments, the cancer is prostate cancer. In certain embodiments, the cancer is breast cancer. In certain embodiments, the cancer is lung cancer. In certain embodiments, the cancer is liver cancer. In certain embodiments, the cancer is bladder cancer. In certain embodiments, the cancer is urinary tract cancer. In certain embodiments, the cancer is eye cancer.

In certain embodiments, the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas (e.g., Burkitt's lymphoma and Non-Hodgkin's lymphoma); benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

In certain embodiments, the cancer is a neuroblastoma, craniopharyngioma, glioma, glioblastoma, schwannoma, astrocytoma, oligodendroglioma, medulloblastoma, pinealoma, hemangioblastoma, retinoblastoma, ependymoma, chordoma, meningioma, medullary carcinoma, small cell lung carcinoma, papillary adenocarcinoma, papillary carcinoma, mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct and gallbladder cancers, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

In certain embodiments, the cancer is a lymphoma. In certain embodiments, the cancer is Burkitt's lymphoma, diffuse large B-cell lymphoma (DLBCL), follicular lymphoma, non-Hodgkin's lymphoma, lymphoid malignancies of T-cell or B-cell origin, peripheral T-cell lymphoma, adult T-cell leukemia-lymphoma, or Waldenstrom's macroglobulinemia.

In certain embodiments, the cancer is a leukemia. In certain embodiments, the cancer is acute leukemia, lymphoblastic leukemia, acute lymphoblastic leukemia, myelogenous leukemia, acute myelogenous leukemia, acute T-cell leukemia, chronic leukemia, chronic lymphocytic leukemia, chronic myelocytic leukemia, chronic myelogenous leukemia, polycythemia vera, multiple myeloma, or erythroleukemia.

In certain embodiments, the cancer is a myelodysplastic and/or myeloproliferative syndrome. In certain embodiments, the cancer is a myelodysplastic syndrome. In certain embodiments, the cancer is a myeloproliferative syndrome.

In certain embodiments, the cancer is a cancer or related myeloproliferative disorder selected from histiocytosis, essential thrombocythemia, myelofibrosis, heavy chain disease, and other malignancies and hyperproliferative disorders of the bladder, breast, colon, lung, ovaries, pancreas, prostate, skin and uterus.

In certain embodiments, the cancer is a B-cell non-Hodgkin's lymphoma, advanced solid tumor, soft tissue sarcoma, INI1-deficient cancer, BAP1-deficient cancer, follicular lymphoma, relapsed/refractory follicular lymphoma, diffuse large B-cell lymphoma, relapsed/refractory diffuse large B-cell lymphoma, non-Hodgkin's lymphoma, pediatric non-Hodgkin's lymphoma, pediatric non-Hodgkin's lymphoma with EZH2, SMARCB1, or SMARCA4 mutation, histiocytic disorder, pediatric histiocytic disorder, pediatric histiocytic disorder with EZH2, SMARCB1, or SMARCA4 mutation, solid tumor with EZH2. SMARCB1, or SAMRCA4 mutation, resistant prostate cancer, relapsed/refractory small-cell lung carcinoma, B-cell lymphoma, relapsed/refractory B-cell lymphoma, adult T-cell leukemia-lymphoma, or advanced diffuse large B-cell lymphoma.

In certain embodiments, the cancer is a malignant rhabdoid tumor, atypical teratoid rhabdoid tumor, epithelioid sarcoma, renal medullary carcinoma, pancreatic undifferentiated rhabdoid carcinoma, schwannoma, epithelioid malignant peripheral nerve sheath tumor, or diffuse intrinsic glioma.

In certain embodiments, the cancer is retinoblastoma multiforme, metastatic castration-resistant prostate cancer, prostate small cell neuroendocrine carcinoma, small-cell lung cancer, triple-negative breast cancer, hepatocellular carcinoma, bladder cancer, or urinary tract cancer.

In certain embodiments, the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma. In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotvpe acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis. Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is a metastatic cancer. In certain embodiments, the cancer is a relapsed and/or refractory cancer.

In certain embodiments, the cancer is ovarian cancer, uterine cancer, gestational trophoblastic disease, endometrial cancer, cervical cancer, embryonal carcinoma, choriocarcinoma, prostate cancer (including hormone insensitive and castrate resistant prostate cancers), testicular tumors (including germ cell testicular cancer/seminoma), cystadenocarcinoma, breast cancer (including estrogen-receptor positive breast cancer), brain tumors (including neuroblastoma, craniopharyngioma, glioma, glioblastoma, schwannoma, astrocytoma, oligodendroglioma, medulloblastoma, and pinealoma), hemangioblastoma, retinoblastoma, ependymoma, chordoma, meningioma, medullary carcinoma, lung cancer (including small cell lung carcinoma, papillary adenocarcinomas, and papillary carcinoma), mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct and gallbladder cancers, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

Methods of Treating Diseases or Disorders Associated with Active PI3K Signaling

A method of treating a disease or disorder associated with active PI3K signaling. The method comprises administering to a subject in need thereof a therapeutically effective amount of a compound described herein, such as a compound of Formula I, to treat the disease or disorder. In certain embodiments, the disease or disorder features hyperactive PI3K signaling.

### Subjects

In certain embodiments, the subject is a human. In certain embodiments, the subject is an adult human. In certain embodiments, the subject is a pediatric human.

### Methods of Inhibiting AKT1 Activity

Another aspect of the invention provides a method of inhibiting AKT1 activity. The method comprises contacting an AKT1 with an effective amount of a compound described herein, such as a compound of Formula I, to thereby inhibit the AKT1 activity.

In certain embodiments, the AKT1 is AKT1 E17K.

In certain embodiments, the AKT1 protein is AKT1 E17K.

### Medical Uses

Another aspect of the invention provides for the use of a compound described herein (such as a compound of Formula I, or other compounds in Section I) in the manufacture of a medicament. In certain embodiments, the medicament is for treating a disorder described herein, such as cancer.

Another aspect of the invention provides for the use of a compound described herein (such as a compound of Formula I, or other compounds in Section I) for treating a medical disorder, such as a medical disorder described herein, such as cancer.

### IV. Combination Therapy

Another aspect of the invention provides for combination therapy. Compounds described herein (such as a compound of Formula I, or other compounds in Section I) or their pharmaceutically acceptable salts may be used in combination with additional therapeutic agents to treat medical disorders, such as an autoimmune disorder or a cancer.

In certain embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In certain embodiments, the method includes co-administering one additional therapeutic agent. In certain embodiments, the method includes co-administering two additional therapeutic agents. In certain embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In certain embodiments, one or more other therapeutic agent and a compound or composition of the invention are administered as a multiple dosage regimen more than 24 hours apart.

### Anti-Cancer Agents

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor ipilumumab has been approved by the United States Food and Drug Administration for treating melanoma. In certain embodiments, the immune checkpoint inhibitor comprises pembrolizumab.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Accordingly, another aspect of the invention provides a method of treating cancer in a patient, where the method comprises administering to the patient in need thereof (i) a therapeutically effective amount of a compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cy clin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52. EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.
In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801. CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137. CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121. AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

In certain embodiments, the second anti-cancer agent is a hormone therapy agent. Exemplary hormone therapy agents include, for example, fulvestrant (faslodex) and other agents that target estrogen receptor and estrogen signaling. In certain embodiments, the second therapeutic agent is a CDK4/6 inhibitor.

### Additional Considerations

The doses and dosage regimen of the active ingredients used in the combination therapy may be determined by an attending clinician. In certain embodiments, the compound described herein (such as a compound of Formula I, or other compounds in Section I) and the additional therapeutic agent(s) are administered in doses commonly employed when such agents are used as monotherapy for treating the disorder. In other embodiments, the compound described herein (such as a compound of Formula I, or other compounds in Section I) and the additional therapeutic agent(s) are administered in doses lower than the doses commonly employed when such agents are used as monotherapy for treating the disorder. In certain embodiments, the compound described herein (such as a compound of Formula I, or other compounds in Section I) and the additional therapeutic agent(s) are present in the same composition, which is suitable for oral administration.

In certain embodiments, the compound described herein (such as a compound of Formula I, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the compound described herein (such as a compound of Formula L, or other compounds in Section I), a pharmaceutically acceptable carrier, vehicle or diluent, and optionally at least one additional therapeutic agent listed above.

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets. e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (such as a compound of Formula I, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc, administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and is not intended to limit the invention.

### Abbreviations

¹H NMR Proton nuclear magnetic resonance spectroscopy
ACN Acetonitrile
AcOH Acetic acid
aq, aqueous
Boc₂O Di-tert-butyl dicarbonate
Cat. Catalytic
CHLOROFORM-d Deuterochloroform
DCE 1,2-Dichloroethane
DCM Dichloromethane
DIEA N,N-Diisopropylethylamine
DMF N,N-Dimethylformamide
DMSO Dimethylsulfoxide
DMSO-d₆ Hexadeuterodimethyl sulfoxide
EDCI 1-(3-Dimethylaminopropyl)-3-ethylcarbodiimide hydrochloride
Et₃N Triethylamine
EtOAc Ethyl acetate
EtOH Ethanol
FA Formic acid
HATU N-[(dimethylamino)-3-oxo-1H-1,2,3-triazolo[4,5-b]pyridin-1-yl-methylene]-N-methylmethanaminium hexafluorophosphate
HPLC High performance liquid chromatography
hr hour
hrs hours
LCMS Liquid Chromatography Mass Spectrometry
MeCN Acetonitrile
MeOH Methanol
METHANOL-d₄ Tetradeuteromethanol
NIS N-Iodosuccinimide
PE Petroleum ether
prep-HPLC Preparative high performance liquid chromatography
Sat, saturated
SEMCl 2-(Trimethylsilyl)ethoxymethyl chloride
TBAF Tetrabutylammonium fluoride
TEA Triethylamine
TFA Trifluoroacetic acid
THF Tetrahy drofuran

### Example 1—Preparation of N-(4-(2-(2-aminopyridin-3-yl)-5-phenyl-3H-imidazo[4,5-b]pyridin-3-yl)benzyl)-4-(3-methyl-5-oxo-4,5-dihydro-1H-pyrazol-1-yl)benzamide (I-1)

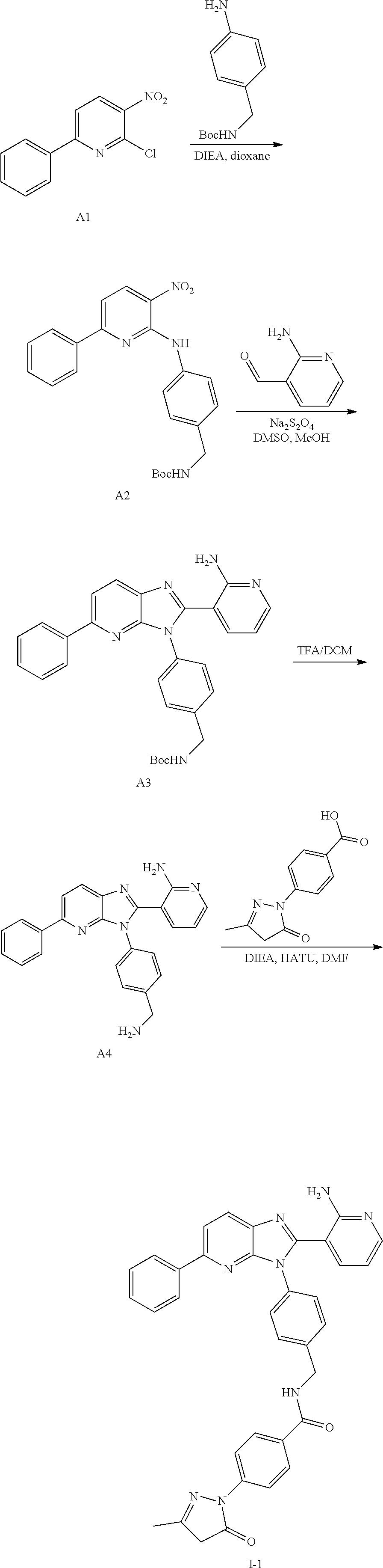

Step 1. To a solution of 2-chloro-3-nitro-6-phenyl-pyridine A1 (15.0 g, 63.9 mmol) and tert-butyl N-[(4-aminophenyl)methyl]carbamate (19.2 g, 86.3 mmol) in 1,4-dioxane (50.0 mL), DIEA (32.0 mL, 192 mmol) was added. The mixture was stirred at 80° C., for 12 hrs. Water (20 mL) was added to the reaction and the resulting mixture was extracted with EtOAc (30.0 mL×3). The combined organic phase was washed with brine (30 mL×3), dried over anhydrous Na2SO4, filtered and concentrated. The residue was purified by flash silica gel chromatography (eluent with 0-30% of EtOAc in PE) to afford tert-butyl N-[[4-[(3-nitro-6-phenyl-2-pyridyl)amino]phenyl]methyl]carbamate A2 (26.0 g, 96.7% yield) as a yellow solid. 1H NMR (DMSO-d6, 400 MHz) δH=10.09 (s, 1H), 8.60 (d, J=8.8 Hz, 1H), 8.11-8.07 (m, 2H), 7.69 (d, J=8.4 Hz, 2H), 7.58 (d, J=8.8 Hz, 1H), 7.55-7.50 (m, 3H), 7.29 (d, J=8.4 Hz, 2H), 4.15 (d, J=6.0 Hz, 2H), 1.41 (m, 9H).

Step 2. To a solution of tert-butyl N-[[4-[(3-nitro-6-phenyl-2-pyridyl)amino]phenyl]methyl]carbamate A2 (30.0 g, 71.3 mmol) and 2-aminopyridine-3-carbaldehyde (11.8 g, 96.3 mmol) in DMSO (100 mL) and methanol (100 mL), Na₂S₂O₄ (37.3 g, 214 mmol) was added under N₂ and the mixture was stirred at 25° C., for 12 hrs. The reaction mixture was concentrated and aq. LiCl (30 mL) was added to the residue. The resulting mixture was extracted with EtOAc (30 mL×3) and the combined organic phase was washed with brine (30 mL), dried over anhydrous Na₂SO₄, filtered and concentrated. The residue was purified by flash silica gel chromatography (eluent with 0-5% of MeOH in DCM) to afford tert-butyl N-[[4-[2-(2-amino-3-pyridyl)-5-phenyl-imidazo[4,5-b]pyridin-3-yl]phenyl]methyl]carbamate A3 (5.90 g, 15.9% yield) as a yellow solid. ¹H NMR (DMSO-d₆, 400 MHz) δ_{H}=8.26 (d, J=8.4 Hz, 1H), 8.04-7.97 (m, 4H), 7.47-7.39 (m, 7H), 7.22-7.18 (m, 1H), 6.99 (s, 2H), 6.44-6.37 (m, 1H), 4.28-4.20 (m, 2H), 1.41 (s, 9H).

Step 3. To a solution of tert-butyl N-[[4-[2-(2-amino-3-pyridyl)-5-phenyl-imidazo[4,5-b]pyridin-3-yl]phenyl]methyl]carbamate A3 (100 mg, 0.203 mmol) in DCM (2.50 mL) TFA (0.500 mL) was added and the mixture was stirred at 25° C., for 12 hrs. The reaction mixture was concentrated directly and the resulting residue was purified by prep-HPLC (Column: Welch Xtimate C18 150*30 mm*5 μm; mobile phase: [water (0.225% FA)-ACN]; B %: 0%-26%; 36 min) to afford 3-(3-(4-(aminomethyl)phenyl)-5-phenyl-3H-imidazo[4,5-b]pyridin-2-yl)pyridin-2-amine A4 (19.1 mg, 24.0% yield) as a light yellow solid. ¹H NMR (METHANOL-d₄, 400 MHz) δ^{H}=8.21 (d, J=8.4 Hz, 1H), 8.04-7.93 (m, 4H), 7.68-7.57 (m, 4H), 7.45-7.35 (m, 4H), 6.48 (dd, J=5.2, 7.6 Hz, 1H), 4.22 (s, 2H). HPLC Rₜ=3.920 min in 8 min chromatography, purity 100%. LCMS Rₜ=2.005 min in 4 min chromatography, purity 99.5%, MS ESI calcd, for 392.17, [M+H]⁺ 393.17, found 393.4.

Step 4. To a solution of 3-(3-(4-(aminomethyl)phenyl)-5-phenyl-3H-imidazo[4,5-b]pyridin-2-yl)pyridin-2-amine A4 (120 mg, 0.306 mmol) in DMF (3.00 mL), 4-(3-methyl-5-oxo-4H-pyrazol-1-yl)benzoic acid (56.0 mg, 0.255 mmol), DIEA (99.0 mg, 0.764 mmol) and HATU (145 mg, 0.382 mmol) were added and the mixture stirred at 25° C., for 2 hrs. The reaction mixture was diluted with DMF (3 mL) and purified by prep-HPLC (column: welch xtimate c18 150*40*10 m; mobile phase: [water (FA)-ACN]; B %: 6%-46%; 36 min) to give N-[[4-[2-(2-amino-3-pyridyl)-5-phenyl-imidazo[4,5-b]pyridin-3-yl]phenyl]methyl]-4-(5-hydroxy-3-methyl-pyrazol-1-yl)benzamide I-1 (33.0 mg, 21.3% yield) as a white solid. ¹H NMR (400 MHz, DMSO-d₆) δ_{H}=11.72 (s, 1H), 9.14 (t, J=6.4 Hz, 1H), 8.29-8.25 (m, 1H), 8.07-7.97 (m, 6H), 7.93-7.85 (m, 2H), 7.52-7.48 (m, 6H), 7.48-7.37 (m, 1H), 7.28-7.27 (m, 1H), 7.06 (s, 2H), 6.46 (dd, J=5.2, 7.6 Hz, 1H), 5.39 (s, 1H), 4.62 (d, J=6.0, 2H), 2.13 (s, 3H). HPLC Rₜ=2.519 min in 8 min chromatography, purity 97.0%. LCMS Rₜ=2.586 min in 4 min chromatography, purity 94.6%, MS ESI calcd. For 592.23 [M+H]⁺ 593.23, found 593.2.

### Example 2—Synthesis of N-(4-(2-(2-aminopyridin-3-yl)-5-phenyl-3H-imidazo[4,5-b]pyridin-3-yl)benzyl)-4-(5-methoxy-3-methyl-1H-pyrazol-1-yl)benzamide (I-2)

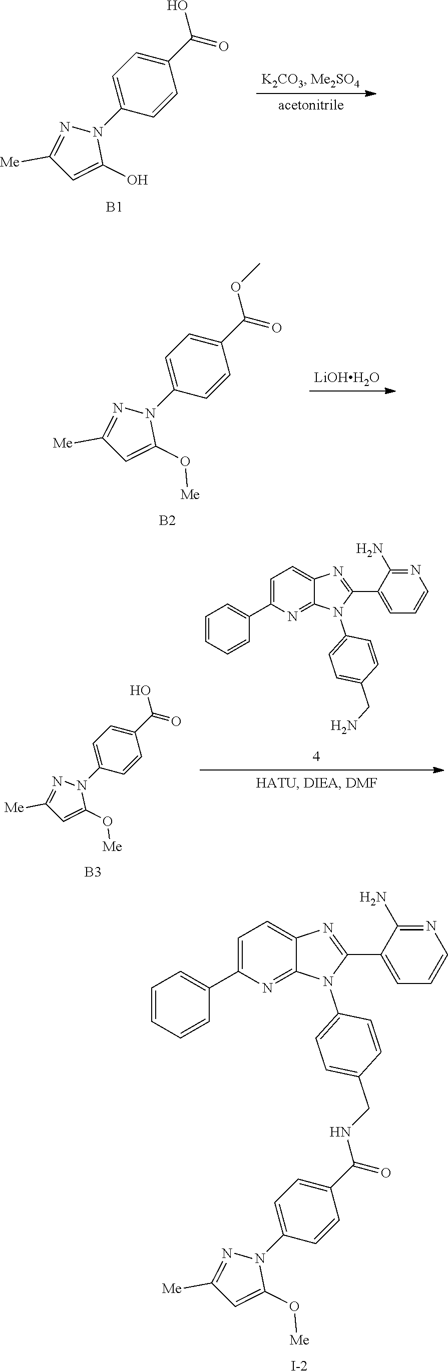

Step 1. To a solution of 4-(5-hydroxy-3-methyl-pyrazol-1-yl)benzoic acid B1 (1.00 g, 4.58 mmol) in MeCN (40.0 mL), dimethyl sulfate (1.70 mL, 18.3 mmol) and K₂CO₃ (3.80 g, 27.5 mmol) were added at 25° C., and the mixture allowed to stir at 70° C., for 7 hrs. The reaction mixture was added to H₂O (20 mL) and subsequently extracted with EtOAc (40 mL×3). The organic layer was dried over Na₂SO₄, filtered and concentrated and the crude material was purified by flash column (0-5% of dichloromethane in methanol) to give methyl 4-(5-methoxy-3-methyl-pyrazol-1-yl)benzoate B2 (240 mg, 21.3% yield) as a yellow solid. ¹H NMR (400 MHz, CHLOROFORM-d) δ=8.06 (d, J=8.8 Hz, 2H), 7.83 (d, J=8.8 Hz, 2H), 5.51 (s, 1H), 3.92 (d, J=12.0 Hz, 6H), 2.27 (s, 3H).

Step 2. To a solution of methyl 4-(5-methoxy-3-methyl-pyrazol-1-yl)benzoate B2 (240 mg, 0.975 mmol) in THF (6.00 mL) and water (6.00 mL), LiOH·H₂O (82.0 mg, 1.95 mmol) was added at 25° C., and the mixture allowed to stir for 2 hrs. The reaction mixture was added to H₂O (10 mL) and subsequently extracted with EtOAc (30 mL×3). The aqueous layer was then acidified to pH 5-6, extracted with EtOAc (30 mL×3) and the organic layer dried over Na₂SO₄, filtered and concentrated to give 4-(5-methoxy-3-methyl-pyrazol-1-yl)benzoic acid B3 (200 mg, 88.4% yield) as a white solid. ¹H NMR (400 MHz, DMSO-d₆) δ=12.93 (s, 1H), 8.04-7.95 (m, 2H), 7.86-7.76 (m, 2H), 5.78 (s, 1H), 3.95 (s, 3H), 2.18 (s, 3H).

Step 3. To a solution of 4-(5-methoxy-3-methyl-pyrazol-1-yl)benzoic acid B3 (50.0 mg, 0.215 mmol) in DMF (2.00 mL), HATU (98.0 mg, 0.258 mmol) and DIEA (0.110 mL, 0.646 mmol) were added at 25° C., and stirred for 15 mins, 3-(3-(4-(aminomethyl)phenyl)-5-phenyl-3H-imidazo[4,5-b]pyridin-2-yl)pyridin-2-amine A4 (84.0 mg, 0.215 mmol) in DMF (2.00 mL) was added and further stirred at 25° C., for 3 hrs. The reaction mixture was purified by prep-HPLC (C18 150×30 mm water (FA)-ACN) to give N-[[4-[2-(2-amino-3-pyridyl)-5-phenyl-imidazo[4,5-b]pyridin-3-yl]phenyl]methyl]-4-(5-methoxy-3-methyl-pyrazol-1-yl)benzamide I-2 (26.0 mg, 19.0% yield) as a white solid. ¹H NMR (400 MHz, DMSO-d₆) δ=9.18 (t, J=6.0 Hz, 1H) 8.26 (d, J=8.4 Hz, 1H), 8.06-7.95 (m, 6H), 7.82-7.74 (m, 2H), 7.52-7.34 (m, 7H), 7.22 (dd, J=7.6, 1.6 Hz, 1H), 6.95 (s, 2H), 6.45-6.38 (m, 1H) 5.77 (s, 1H), 4.62 (d, J=5.6 Hz, 2H), 3.95 (s, 3H) 2.18 (s, 3H). HPLC Rₜ=5.668 min in 8 min chromatography, Ultimate C18 3.0*50 mm, 3 m, purity 96.3%. LCMS Rₜ=2.943 min in 4 min chromatography, Xtimate C18 2.1*30 mm, 3 m, purity 98.6%, MS ESI calcd. For 606.25. [M+H]⁺ 607.25, found 607.2.

### Example 3. Synthesis of N-(4-(2-(2-aminopyridin-3-yl)-5-phenyl-3H-imidazo[4,5-b]pyridin-3-yl)benzyl)-3-(5-hydroxy-3-methyl-1H-pyrazol-1-yl)benzamide (I-3)

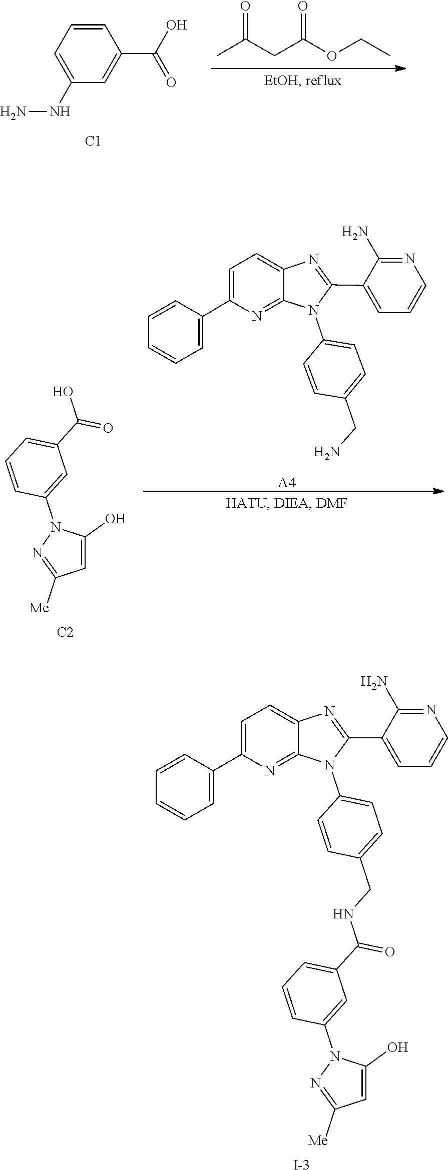

Step 1. To a mixture of 3-hydrazinylbenzoic acid C₁ (200 mg, 1.31 mmol) in ethanol (5.00 mL), ethyl 3-oxobutanoate (171 mg, 1.31 mmol) was added and the mixture stirred at 90° C., for 12 hrs. The reaction mixture was directly concentrated to give 3-(5-hydroxy-3-methyl-1H-pyrazol-1-yl)benzoic acid C2 (300 mg, 51.7% yield) as a yellow solid. ¹H NMR (400 MHz, DMSO-d₆) δ_{H}=8.33-8.28 (m, 1H), 8.04-7.90 (m, 2H), 7.81-7.78 (m, 1H), 7.62-7.52 (m, 2H), 2.17 (s, 3H).

Step 2. To a solution of 3-(3-(4-(aminomethyl)phenyl)-5-phenyl-3H-imidazo[4,5-b]pyridin-2-yl)pyridin-2-amine A4 (300 mg, 0.760 mmol) in DMF (5.00 mL), 3-(5-hydroxy-3-methyl-1H-pyrazol-1-yl)benzoic acid C₂ (167 mg, 0.760 mmol), DIEA (296 mg, 2.29 mmol), and then HATU (349 mg, 0.91 mmol) were added, and the mixture stirred at 25° C. for 12 hrs. The reaction mixture was poured into water (30 mL), extracted with EtOAc (20 mL×3) and the combined organic phase was washed with brine (30 mL×5), dried over anhydrous Na₂SO₄, filtered and the filtrate was concentrated. The crude product was purified by combin-flash column (MeOH in DCM, 2%) and then purified by prep-HPLC (Column: Welch Xtimate C18 150*30 mm*5 m; mobile phase: [water (FA)-ACN]; B %: 8%-48%; 25 min) to give N-(4-(2-(2-aminopyridin-3-yl)-5-phenyl-3H-imidazo[4,5-b]pyridin-3-yl)benzyl)-3-(5-hydroxy-3-methyl-1H-pyrazol-1-yl)benzamide I-3 (10.3 mg, 2.20% yield) as a white solid. ¹H NMR (400 MHz, DMSO-d₆) δ_{H}=9.24 (t, J=6.0 Hz, 1H), 8.30-8.21 (m, 2H), 8.07-7.86 (m, 5H), 7.79-7.72 (m, 1H), 7.56-7.51 (m, 1H), 7.49-7.42 (m, 6H), 7.41-7.35 (m, 1H), 7.22 (dd, J=1.6, 7.6 Hz, 1H), 6.95 (s, 2H), 6.42 (dd, J=4.4, 7.2 Hz, 1H), 5.38 (brs, 1H), 4.61 (d, J=6.0 Hz, 2H), 2.13 (s, 3H). HPLC Rₜ=2.431 min in 8 min chromatography, purity 98.9%. LCMS Rₜ=2.444 min in 4 min; Agilent PoroShell 120 EC-C18 2.7 μm 3.0*50 mm; purity 98.6%, MS ESI calcd, for 592.23, [M+H]⁺ 593.23, found 593.2.

### Example 4. Synthesis of N-[[4-[2-(2-amino-3-pyridyl)-5-phenyl-imidazo[4,5-b]pyridin-3-yl]phenyl]methyl]-2-fluoro-4-(5-hydroxy-3-methyl-pyrazol-1-yl)benzamide (I-4)

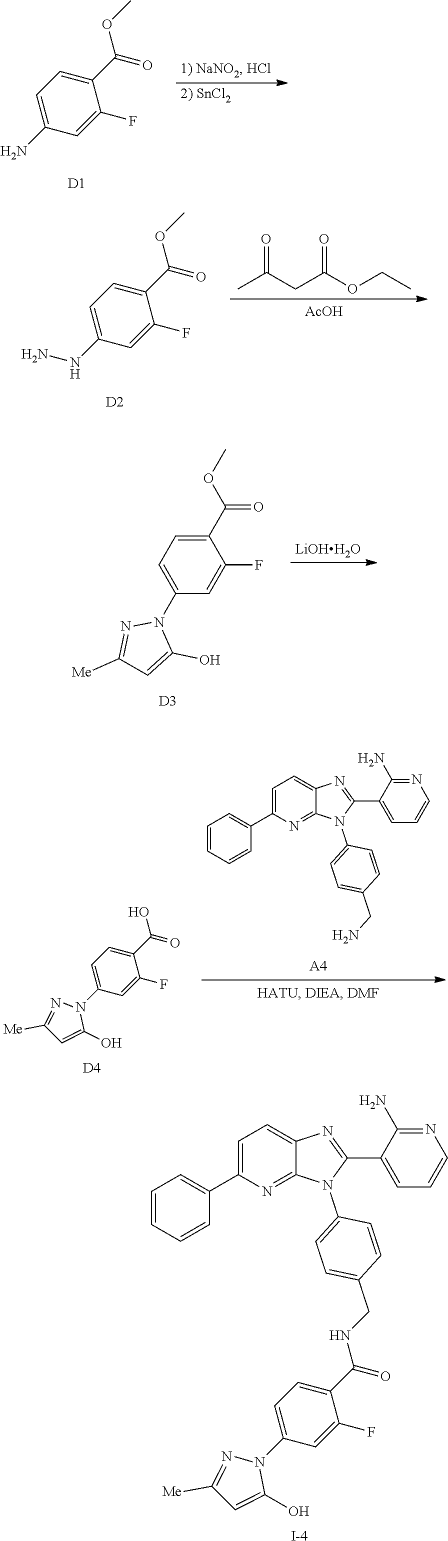

Step 1. To a solution of methyl 4-amino-2-fluoro-benzoate D1 (3.00 g, 17.7 mmol) in 3M HCl (20.0 mL), a solution of NaNO₂ (1.42 g, 20.6 mmol) in water (10.0 mL) was added dropwise at 0° C. The mixture was allowed to stir at 0° C., for 20 mins. A solution of SnCl₂ (10.0 g, 44.3 mmol) in 3M HCl (20.0 mL) was then added to the reaction mixture at 0° C., and the mixture stirred at 5° C. under N₂ for 1 hr. The reaction mixture was washed with EtOAc (20 mL) and the aqueous phase adjusted to pH 9 with a 5M NaOH solution. The mixture was extracted with DCM (50 mL×2) and the combined organic layers were dried over anhydrous sodium sulfate, filtered and the filtrate was concentrated. The crude material was purified by flash chromatography on silica gel (45-50% of EtOAc in PE) to afford methyl 2-fluoro-4-hydrazino-benzoate D2 (400 mg, 12.3% yield) as a white solid. ¹H NMR (400 MHz, DMSO-d₆) δ_{H}=7.89 (s, 1H), 7.59 (t, J=8.8 Hz, 1H), 6.64-6.33 (m, 2H), 4.30 (d, J=3.2 Hz, 2H), 3.72 (s, 3H).

Step 2. To a solution of methyl 2-fluoro-4-hydrazino-benzoate D2 (300 mg, 1.63 mmol) in ethanol (3.00 mL) and acetic acid (3.00 mL), ethyl 3-oxobutanoate (0.210 mL, 1.63 mmol) was added at 25° C. The mixture was stirred at 90° C., for 12 hrs. The reaction mixture was concentrated directly and the residue was purified by flash silica gel chromatography (eluent of 0-10% MeOH in DCM) to afford methyl 2-fluoro-4-(5-hydroxy-3-methyl-pyrazol-1-yl)benzoate D3 (300 mg, 73.6% yield) as a yellow solid. ¹H NMR (400 MHz, DMSO-d₆) δ=12.18 (s, 1H), 8.04-7.91 (m, 1H), 7.85-7.57 (m, 2H), 3.85 (s, 3H), 2.13 (s, 3H).

Step 3. To a stirred solution of methyl 2-fluoro-4-(5-hydroxy-3-methyl-pyrazol-1-yl)benzoate D3 (200 mg, 0.800 mmol) in THF (1.00 mL) and water (0.50 mL). LiOH·H₂O (64.0 mg, 1.60 mmol) was added and the reaction mixture stirred at 25° C. under N₂ for 2 hrs. The reaction mixture was concentrated and the crude product was purified by prep-HPLC (column: Welch Xtimate C18 150*30 mm*5 m, method: water (FA)-CAN, begin B: 17, end B: 47) to afford 2-fluoro-4-(5-hydroxy-3-methyl-pyrazol-1-yl)benzoic acid D4 (150 mg, 79.5% yield) as a white solid. ¹H NMR (400 MHz, DMSO-d₆) δ_{H}=13.06 (s, 1H), 8.07-7.87 (m, 1H), 7.82-7.58 (m, 2H), 5.39 (s, 1H), 2.13 (s, 3H).

Step 4. To a stirred solution of 2-fluoro-4-(5-hydroxy-3-methyl-pyrazol-1-yl)benzoic acid D4 (33.0 mg, 0.140 mmol) in DMF (2.00 mL), HATU (59.0 mg, 0.150 mmol) and DIEA (91.0 mg, 0.70 mmol) were added and the reaction mixture was stirred at 25° C., for 10 mins, 3-(3-(4-(Aminomethyl)phenyl)-5-phenyl-3H-imidazo[4,5-b]pyridin-2-yl)pyridin-2-amine A4 (55.0 mg, 0.140 mmol) was then added to the reaction mixture and the reaction stirred at 25° C., for 2 hrs. The reaction mixture was diluted with EtOAc (10 mL), washed with water (10 mL), brine (10 mL), dried over anhydrous sodium sulfate, filtered and concentrated. The crude product was purified by prep-HPLC (column: Welch Xtimate C18 150*30 mm*5 μm, method: water (FA)-CAN, begin B: 28, end B: 58) to afford N-[[4-[2-(2-amino-3-pyridyl)-5-phenyl-imidazo[4,5-b]pyridin-3-yl]phenyl]methyl]-2-fluoro-4-(5-hydroxy-3-methyl-pyrazol-1-yl)benzamide I-4 (4.40 mg, 5.01% yield) as an off-white solid. ¹H NMR (400 MHz, DMSO-d₆) δ=8.89 (s, 1H), 8.27 (d, J=8.4 Hz, 1H), 8.10-7.91 (m, 4H), 7.84-7.64 (m, 3H), 7.62-7.29 (m, 8H), 7.28-7.11 (m, 1H), 6.96 (s, 2H), 6.42 (dd, J=4.8, 7.6 Hz, 1H), 5.30 (s, 1H), 4.60 (d, J=6.0 Hz, 2H), 2.11 (s, 3H). HPLC Rₜ=3.937 min in 8 min chromatography, purity 98.2%. LCMS Rₜ=1.003 min in 2 min chromatography, purity 100%, MS ESI calcd, for 610.22 [M+H]⁺ 611.22, found 611.1.

### Example 5. Synthesis of 2-(3-((4-(2-(2-aminopyridin-3-yl)-5-phenyl-3H-imidazo[4,5-b]pyridin-3-yl)benzyl)carbamoyl)phenyl)propanoic acid (I-5)

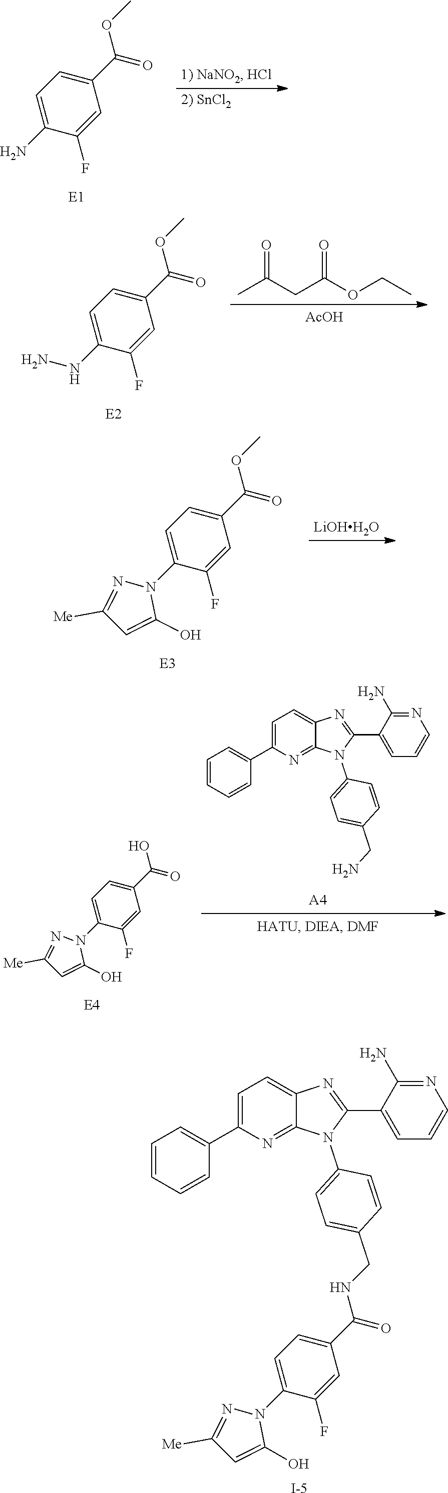

Step 1. To a solution of 3M HCl (20.0 mL), methyl 4-amino-3-fluoro-benzoate E1 (4.00 g, 23.6 mmol) was added dropwise at 0° C. A solution of NaNO₂ (1.89 g, 27.4 mmol) in 3N HCl (5.00 mL) was then added to the reaction mixture and the reaction mixture was stirred at 0° C. under N₂ for 20 min. A solution of SnCl₂ (13.4 g, 59.1 mmol) in 3M HCl (10.0 mL) was then added to the reaction mixture dropwise. The pH of the reaction mixture was adjusted to pH 7-8 with a 5M NaOH solution, H₂O (20 mL) was added and extracted with EtOAc (30 mL×3). Then organic layer was dried over Na₂SO₄, filtered and concentrated in vacuum. The crude material was purified by flash column (0-50% of EtOAc in PE) to give methyl 3-fluoro-4-hydrazino-benzoate E2 (160 mg, 14.7% yield) as a brown solid. ¹H NMR (400 MHz, DMSO-d₆) δ_{H}=10.92-10.43 (m, 1H), 9.25-8.69 (m, 1H), 7.78-7.64 (m, 1H), 7.23 (t, J=8.4 Hz, 1H), 3.81 (s, 3H), 3.59-3.41 (m, 2H). ¹⁹F NMR (400 MHz, DMSO-d₆), δ_{H}=−129.246.

Step 2. To a mixture of methyl 3-fluoro-4-hydrazino-benzoate E2 (780 mg, 4.24 mmol) in acetic acid (10.0 mL), ethyl 3-oxobutanoate (0.540 mL, 4.24 mmol) was added and the mixture stirred at 90° C., for 2 hrs. The mixture was subsequently concentrated, H₂O (20 mL) then added, and the whole extracted with EtOAc (30 mL×3). The organic layer was dried over Na₂SO₄, and concentrated in vacuum to provide the crude material. The crude material was purified by flash column (0-50% of EtOAc in PE) to give methyl 3-fluoro-4-(5-hydroxy-3-methyl-pyrazol-1-yl)benzoate E3 (300 mg, 28.3% yield) as a brown solid. ¹H NMR (400 MHz, DMSO-d₆)⁶H=7.92-7.81 (m, 2H), 7.68-7.57 (m, 1H), 5.37 (s, 1H), 3.90-3.86 (m, 3H), 2.14-2.08 (m, 3H). ¹⁹F NMR (400 MHz, DMSO-d₆) δ=−118.865.

Step 3. To the solution of methyl 3-fluoro-4-(5-hydroxy-3-methyl-pyrazol-1-yl)benzoate E3 (280 mg, 1.12 mmol) in THF (2.00 mL) and water (2.00 mL), LiOH·H₂O (94.0 mg, 2.24 mmol) was added at 25° C., and the reaction stirred for 2 hrs. Water (10 mL) was then added and the whole extracted with EtOAc (30 mL×3). The aqueous layer was purified by HPLC (column: Welch Xtimate C18 150*30 mm*5 m; mobile phase: water (FA)-CAN; B %: 20%-50%, 25 min) to give 3-fluoro-4-(5-hydroxy-3-methyl-pyrazol-1-yl)benzoic acid E4 (160 mg, 60.5% yield) as a white solid. ¹H NMR (400 MHz, DMSO-d₆) δ_{H}=7.76-7.87 (m, 2H) 7.59 (t, J=7.2 Hz, 1H) 5.36 (s, 1H), 2.11 (s, 3H). ¹⁹F NMR (400 MHz, DMSO-d₆) δ=−119.437.

Step 4. To the solution of 3-fluoro-4-(5-hydroxy-3-methyl-pyrazol-1-yl)benzoic acid E4 (50.0 mg, 0.210 mmol) in DMF (2.00 mL), HATU (97.0 mg, 0.250 mmol) and DIEA (0.110 mL, 0.640 mmol) were added at 25° C., and the mixture stirred for 15 mins, 3-(3-(4-(Aminomethyl)phenyl)-5-phenyl-3H-imidazo[4,5-b]pyridin-2-yl)pyridin-2-amine A4 (83.0 mg, 0.210 mmol) in DMF (2.00 mL) was then added at 25° C., and allowed to stir for 3 hrs. The reaction mixture was purified by HPLC Welch Xtimate C18 150*30 mm*5 m, water (FA)-CAN; B %: 6%-36%, 25 min) to give N-[[4-[2-(2-amino-3-pyridyl)-5-phenyl-imidazo[4,5-b]pyridin-3-yl]phenyl]methyl]-3-fluoro-4-(5-hydroxy-3-methyl-pyrazol-1-yl)benzamide I-5 (38.0 mg, 22.7% yield) as a yellow solid. ¹H NMR (400 MHz, DMSO-d₆) δH=11.35 (s, 1H) 9.31 (t, J=5.6 Hz, 1H), 8.27 (d, J=8.4 Hz, 1H), 8.07-7.95 (m, 4H), 7.93-7.82 (m, 2H), 7.66-7.32 (m, 8H), 7.22 (dd, J=7.6, 1.6 Hz, 1H), 6.94 (s, 2H), 6.42 (dd. J=7.6, 4.8 Hz, 1H), 5.37 (s, 1H), 4.63 (d, J=6.0 Hz, 2H), 2.10 (s, 3H). ¹⁹F NMR (400 MHz, DMSO-d₆) δ=−119.848. HPLC Rₜ=4.953 min in 8 min chromatography, Ultimate C18 3.0*50 mm, 3 μm, purity 93.7%. LCMS Rₜ=2.623 min in 4 min chromatography, Xtimate C18 2.1*30 mm, 3 μm, purity 98.6%, MS ESI calcd. For 610.22, [M+H]⁺ 611.22, found 611.4.

### Example 6. Synthesis of N-(4-(2-(2-aminopyridin-3-yl)-5-phenyl-3H-imidazo[4,5-b]pyridin-3-yl)benzyl)-4-(5-hydroxy-3-(trifluoromethyl)-1H-pyrazol-1-yl)benzamide (I-6)

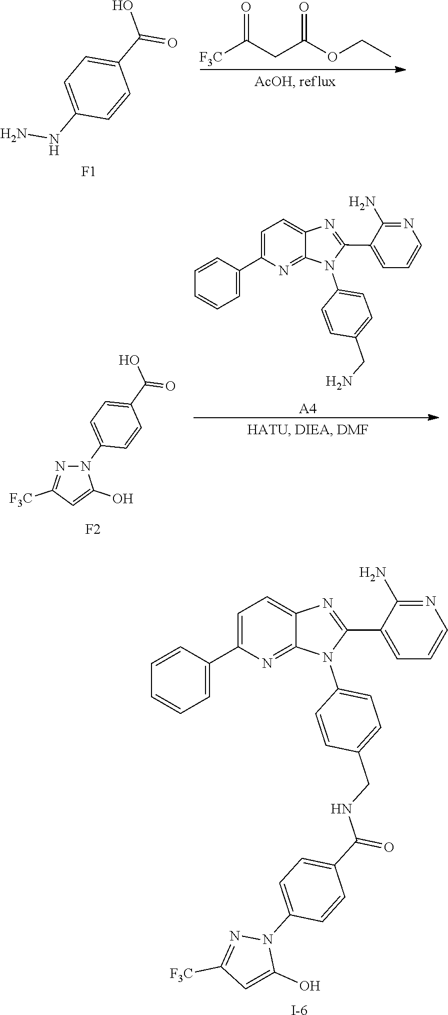

Step 1. To a solution of 4-hydrazineylbenzoic acid F1 (500 mg, 3.29 mmol) in acetic acid (5.00 mL), ethyl 4,4,4-trifluoro-3-oxo-butanoate (605 mg, 3.29 mmol) was added and the mixture stirred at 110° C., for 12 hrs. The reaction mixture was concentrated directly to give 4-(5-hydroxy-3-(trifluoromethyl)-1H-pyrazol-1-yl)benzoic acid F2 (660 mg, crude) as a yellow solid, which was used in the next step directly. ¹H NMR (DMSO-d₆, 400 MHz) δ_{H}=12.93 (brs, 2H), 8.07 (d, J=8.8 Hz, 2H), 7.91 (d, J=8.8 Hz, 2H), 5.97 (s, 1H).

Step 2. To a solution of 3-(3-(4-(aminomethyl)phenyl)-5-phenyl-3H-imidazo[4,5-b]pyridin-2-yl)pyridin-2-amine A4 (150 mg, 0.380 mmol) and 4-(5-hydroxy-3-(trifluoromethyl)-1H-pyrazol-1-yl)benzoic acid F2 (156 mg, 0.570 mmol) in DMF (5.00 mL), DIEA (148 mg, 1.15 mmol) and HATU (218 mg, 0.570 mmol) were added and the mixture stirred at 25° C., for 6 hrs. Water (5 mL) was added to the reaction mixture and the resulting mixture was extracted with EtOAc (10 mL×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (FA)-ACN]; B %: 26%-66%; 20 min) to afford N-(4-(2-(2-aminopyridin-3-yl)-5-phenyl-3H-imidazo[4,5-b]pyridin-3-yl)benzyl)-4-(5-hydroxy-3-(trifluoromethyl)-1H-pyrazol-1-yl)benzamide I-6 (24.7 mg, 9.63% yield) as a light yellow solid. 1H NMR (DMSO-d₆, 400 MHz) δ_{H}=9.22 (t, J=6.0 Hz, 1H), 8.26 (d, J=8.4 Hz, 1H), 8.06-7.97 (m, 6H), 7.95-7.90 (m, 2H), 7.52-7.44 (m, 6H), 7.41-7.36 (m, 1H), 7.25-7.20 (m, 1H), 6.95 (s, 2H), 6.42 (dd, J=4.8, 7.6 Hz, 1H), 5.81 (s, 1H), 4.63 (d, J=6.0 Hz, 2H). HPLC Rₜ═3.445 min in 8 min chromatography, purity 96.4%. LCMS Rₜ=1.441 min in 2 min chromatography, purity 96.3%, MS ESI calcd, for 646.21, [M+H]⁺ 647.21, found 647.0.

### Example 7. Synthesis of N-(4-(2-(2-aminopyridin-3-yl)-5-phenyl-3H-imidazo[4,5-b]pyridin-3-yl)benzyl)-3-(5-hydroxy-3-(trifluoromethyl)-1H-pyrazol-1-yl)benzamide (I-7)

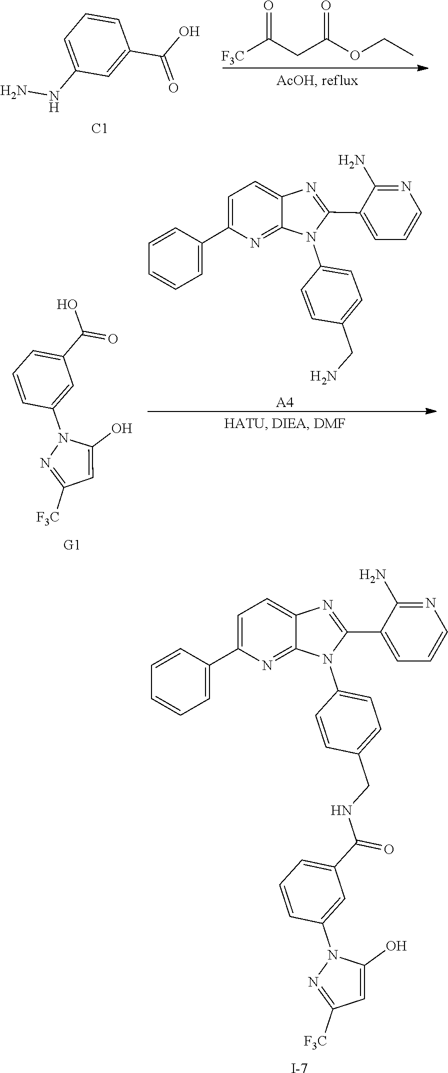

Step 1. To a solution of 3-hydrazineylbenzoic acid C1 (200 mg, 1.31 mmol) in acetic acid (5.00 mL), 4,4,4-trifluoro-3-oxo-butanoate (242 mg, 1.31 mmol) was added, and the mixture stirred at 110° C., for 12 hrs. The reaction mixture was concentrated directly to give 3-(5-hydroxy-3-(trifluoromethyl)-1H-pyrazol-1-yl)benzoic acid G1 (260 mg, crude) as a yellow solid, which was used in the next step directly. ¹H NMR (DMSO-d₆, 400 MHz) δ_{H}=13.34-12.61 (m, 2H), 8.30 (s, 1H), 8.03-7.90 (m, 3H), 5.96 (s, 1H).

Step 2. To a solution of 3-(3-(4-(aminomethyl)phenyl)-5-phenyl-3H-imidazo[4,5-b]pyridin-2-yl)pyridin-2-amine A4 (150 mg, 0.382 mmol) and 3-(5-hydroxy-3-(trifluoromethyl)-1H-pyrazol-1-yl)benzoic acid G1 (156 mg, 0.573 mmol) in DMF (5.00 mL), DIEA (148 mg, 1.15 mmol) and HATU (218 mg, 0.573 mmol) were added and the mixture stirred at 25° C., for 6 hrs. Water (5 mL) was subsequently added and the resulting mixture was extracted with EtOAc (10 mL×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (FA)-ACN]; B %: 26%-66%; 20 min) to afford N-(4-(2-(2-aminopyridin-3-yl)-5-phenyl-3H-imidazo[4,5-b]pyridin-3-yl)benzyl)-3-(5-hydroxy-3-(trifluoromethyl)-1H-pyrazol-1-yl)benzamide I-7 (13.8 mg, 5.52% yield) as a yellow solid. ¹H NMR (DMSO-d₆, 400 MHz) δ_{H}=9.32 (t, J=6.0 Hz, 1H), 8.30-8.24 (m, 2H), 8.04-7.91 (m, 6H), 7.63 (t, J=8.0 Hz, 1H), 7.52-7.43 (m, 6H), 7.41-7.35 (m, 1H), 7.25-7.19 (m, 1H), 6.96 (s, 2H), 6.42 (dd, J=4.8, 7.6 Hz, 1H), 5.94 (s, 1H), 4.63 (d, J=5.6 Hz, 2H). HPLC Rₜ=2.994 min in 8 min chromatography, purity 98.9%. LCMS Rₜ=1.442 min in 2 min chromatography, purity 98.7%, MS ESI calcd, for 646.21, [M+H]⁺ 647.21, found 647.0.

### Example 8. Synthesis of N-(4-(2-(2-aminopyridin-3-yl)-5-phenyl-3H-imidazo[4,5-b]pyridin-3-yl)benzyl)-4-(5-hydroxy-3-phenyl-1H-pyrazol-1-yl)benzamide (I-8)

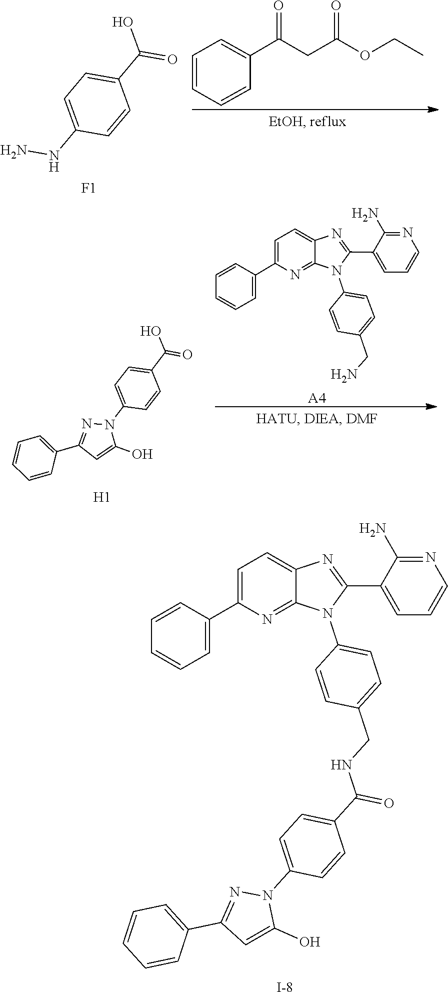

Step 1. To a solution of 4-hydrazineylbenzoic acid F1 (1.00 g, 6.57 mmol) in ethanol (10.0 mL), ethyl 3-oxo-3-phenylpropanoate (1.26 g, 6.57 mmol) was added at 25° C., and the mixture stirred at 90° C., for 2 hrs. The reaction mixture was concentrated to provide 4-(5-hydroxy-3-phenyl-1H-pyrazol-1-yl)benzoic acid H1 (2.10 g, crude) as a brown solid, which was used in the next step directly.

Step 2. To a solution of 3-(3-(4-(aminomethyl)phenyl)-5-phenyl-3H-imidazo[4,5-b]pyridin-2-yl)pyridin-2-amine A4 (100 mg, 0.255 mmol) in DCM (5.00 mL), 4-(5-hydroxy-3-phenyl-1H-pyrazol-1-yl)benzoic acid H1 (143 mg, 0.510 mmol), DIEA (99.0 mg, 0.764 mmol) and HATU (145 mg, 0.382 mmol) were added at 25° C., and the mixture stirred for 16 hrs. The reaction mixture was concentrated directly and the resultant residue was purified by flash silica gel chromatography (eluent with 0-10% of MeOH in DCM) and then purified by prep-HPLC (Column: Xtimate C18 150*30 mm*5 μm; Condition: water (HCl)-ACN; begin b: 22-62%) to afford N-(4-(2-(2-aminopyridin-3-yl)-5-phenyl-3H-imidazo[4,5-b]pyridin-3-yl)benzyl)-4-(5-hydroxy-3-phenyl-1H-pyrazol-1-yl)benzamide I-8 (3.30 mg, 1.96% yield) as a yellow solid. ¹H NMR (400 MHz, DMSO-d₆) δ=9.20 (t, J=6.0 Hz, 1H), 8.33 (d, J=8.4 Hz, 1H), 8.09-8.02 (m, 7H), 8.01-7.97 (m, 2H), 7.87-7.83 (m, 2H), 7.74 (d, J=4.4 Hz, 1H), 7.57-7.32 (m, 11H), 6.79 (t, J=6.4 Hz, 1H), 6.06 (s, 1H), 4.63 (d, J=6.0 Hz, 2H). HPLC Rₜ=4.473 min in 8 min chromatography, purity 99.2%. LCMS Rₜ=1.388 min in 2.5 min chromatography, purity 98.3%, MS ESI calcd, for 654.25 [M+H]⁺ 655.25, found 655.2.

### Example 9. Methyl 2-(1-(4-((4-(2-(2-aminopyridin-3-yl)-5-phenyl-3H-imidazo[4,5-b]pyridin-3-yl)benzyl)carbamoyl)phenyl)-5-hydroxy-1H-pyrazol-3-yl)acetate (I-9)

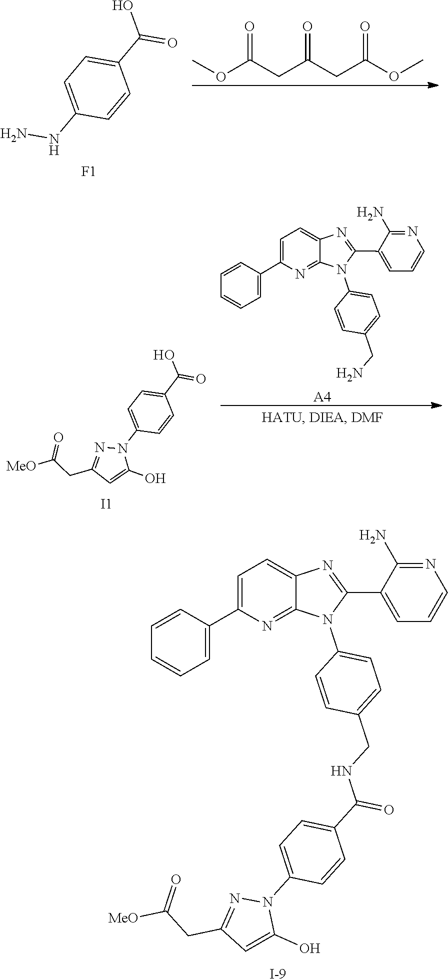

Step 1. To a solution of 4-hydrazineylbenzoic acid F1 (500 mg, 3.29 mmol) in ethanol (5.00 mL), dimethyl 3-oxopentanedioate (572 mg, 3.29 mmol) and acetic acid (5.00 mL) were added at 25° C., and the mixture stirred at 80° C., for 12 hrs. The reaction mixture was subsequently concentrated directly and the crude product was purified by prep-HPLC (column: Xtimate C18 150*40 mm*10 μm; mobile phase: [water (FA)-ACN]; B %: 0%-38%, 25 min) to give 4-(5-hydroxy-3-(2-methoxy-2-oxoethyl)-1H-pyrazol-1-yl)benzoic acid I1 (300 mg, 33.1% yield) as a white solid. ¹H NMR (400 MHz, DMSO-d₆) δ_{H}=12.89 (s, 1H), 12.01 (s, 1H), 8.00 (d, J=8.8 Hz, 2H), 7.89 (d, J=8.8 Hz, 2H), 5.53 (s, 1H), 3.64 (s, 3H), 3.59 (s, 2H). LCMS Rₜ=2.380 min in 7 min chromatography, purity 98.9%, MS ESI calcd, for 276.07 [M+H]⁺ 277.07, found 277.2.

Step 2. To a solution of 3-(3-(4-(aminomethyl)phenyl)-5-phenyl-3H-imidazo[4,5-b]pyridin-2-yl)pyridin-2-amine A4 (120 mg, 0.300 mmol) in DMF (1.00 mL), 4-(5-hydroxy-3-(2-methoxy-2-oxoethyl)-1H-pyrazol-1-yl)benzoic acid I1 (101 mg, 0.360 mmol) and DIEA (198 mg, 1.53 mmol) were added and the mixture stirred at 25° C., for 15 mins. HATU (174 mg, 0.450 mmol) was subsequently added at 0° C., and the resulting mixture stirred at 25° C. for 2 hrs. The mixture was diluted with H₂O (10 mL), extracted with EtOAc (10 mL×3), washed with brine (10 mL) and the organic layer was dried over Na₂SO₄, filtered and concentrated. The crude product was purified by prep-HPLC (column: Xtimate C18 150*40 mm*10 μm; mobile phase: water (FA)-ACN; B %: 8%-48%, 25 min) to give methyl 2-(1-(4-((4-(2-(2-aminopyridin-3-yl)-5-phenyl-3H-imidazo[4,5-b]pyridin-3-yl)benzyl)carbamoyl)phenyl)-5-hydroxy-1H-pyrazol-3-yl)acetate I-9 (24.1 mg, 11.9% yield) as a white solid. ¹H NMR (400 MHz, DMSO-d₆) δ_{H}=9.15 (d, J=6.8 Hz, 1H), 8.27 (d, J=8.4 Hz, 1H), 8.06-7.95 (m, 6H), 7.89 (d, J=7.6 Hz, 2H), 7.53-7.43 (m, 6H), 7.43-7.36 (m, 1H), 7.22 (dd, J=7.6, 1.6 Hz, 1H), 6.95 (s, 2H), 6.42 (dd, J=7.6, 4.8 Hz, 1H), 5.47 (s, 1H), 4.62 (d, J=5.6 Hz, 2H), 3.64 (s, 3H), 3.57 (s, 2H). HPLC Rₜ=4.353 min in 8 min chromatography, purity 98.6%. LCMS Rₜ=2.607 min in 4 min chromatography, purity 95.8%, MS ESI calcd, for 650.24 [M+H]⁺ 651.24, found 651.3.

### Example 10. Synthesis of 2-(1-(4-((4-(2-(2-aminopyridin-3-yl)-5-phenyl-3H-imidazo[4,5-b]pyridin-3-yl)benzyl)carbamoyl)phenyl)-5-hydroxy-1H-pyrazol-3-yl)acetic acid (I-10)

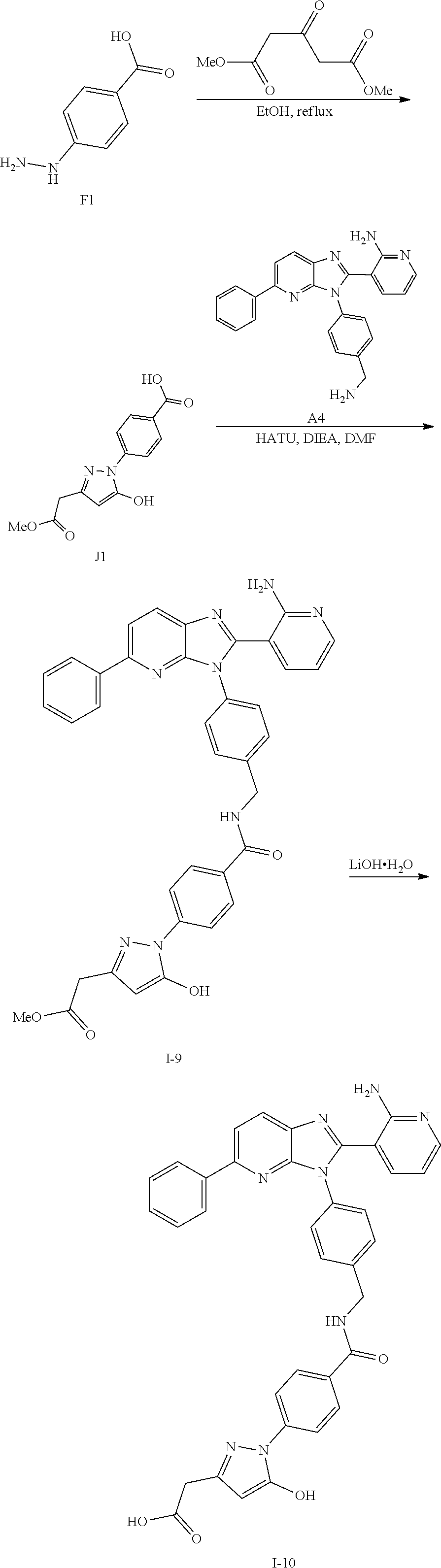

Step 1. To a solution of 4-hydrazineylbenzoic acid F1 (500 mg, 3.29 mmol) in ethanol (10.0 mL), dimethyl 3-oxopentanedioate (630 mg, 3.61 mmol) was added at 25° C. and the mixture stirred at 90° C., for 2 hrs. The reaction mixture was concentrated to give 4-(5-hydroxy-3-(2-methoxy-2-oxoethyl)-1H-pyrazol-1-yl)benzoic acid J1 (763 mg, 84.1% yield) as a yellow solid, which was used in the next step directly.

Step 2. To a solution of 3-(3-(4-(aminomethyl)phenyl)-5-phenyl-3H-imidazo[4,5-b]pyridin-2-yl)pyridin-2-amine A4 (200 mg, 0.510 mmol) in DCM (8.00 mL), 4-(5-hydroxy-3-(2-methoxy-2-oxoethyl)-1H-pyrazol-1-yl)benzoic acid J1 (211 mg, 0.764 mmol), DIEA (198 mg, 1.53 mmol) and HATU (291 mg, 0.764 mmol) were added at 25° C., and the mixture stirred for 16 hrs. The reaction mixture was concentrated directly and the residue was purified by flash silica gel chromatography (eluent with 0-63% of EtOAc in PE) and subsequently purified by prep-HPLC (Column: Xtimate C18 150*30 mm*5 μm; Condition: water (HCl)-ACN; begin B: 22-62%) to give methyl 2-(1-(4-((4-(2-(2-aminopyridin-3-yl)-5-phenyl-3H-imidazo[4,5-b]pyridin-3-yl)benzyl)carbamoyl)phenyl)-5-hydroxy-1H-pyrazol-3-yl)acetate I-9 (133 mg, 40.1% yield) as a brown solid. LCMS Rₜ=0.527 min in 1.0 min chromatography, purity 64.9%, MS ESI calcd, for 650.24 [M+H]⁺ 651.24, found 651.4.

Step 3. To a solution of methyl 2-(1-(4-((4-(2-(2-aminopyridin-3-yl)-5-phenyl-3H-imidazo[4,5-b]pyridin-3-yl)benzyl)carbamoyl)phenyl)-5-hydroxy-1H-pyrazol-3-yl)acetate 9 (100 mg, 0.154 mmol) in THF (2.00 mL) and water (2.00 mL), LiOH·H₂O (13.0 mg, 0.307 mmol) was added at 25° C., and the mixture stirred for 2 hrs and subsequently concentrated to remove THF. The residue was purified by prep-HPLC (Column: Xtimate C18 150*30 mm*5 μm; Condition: water (HCl)-ACN; begin B: 10-50%) to give 2-(1-(4-((4-(2-(2-aminopyridin-3-yl)-5-phenyl-3H-imidazo[4,5-b]pyridin-3-yl)benzyl)carbamoyl)phenyl)-5-hydroxy-1H-pyrazol-3-yl)acetic acid I-10 (34.0 mg, 34.8% yield) as a yellow solid. ¹H NMR (400 MHz, DMSO-d₆) δ=9.19 (t, J=6.0 Hz, 1H), 8.35 (d, J=8.4 Hz, 2H), 8.12-7.98 (m, 7H), 7.90-7.84 (m, 3H), 7.58-7.38 (m, 8H), 6.89 (t, J=7.2 Hz, 1H), 5.53 (s, 1H), 4.61 (d, J=6.0 Hz, 2H), 3.47 (s, 2H). HPLC Rₜ=2.327 min in 8.0 min chromatography, purity 99.2%. LCMS Rₜ=1.179 min in 2.5 min chromatography, purity 99.1%, MS ESI calcd, for 636.22 [M+H]⁺ 637.22, found 637.2.

### Example 11. Synthesis of 1-(4-((4-(2-(2-aminopyridin-3-yl)-5-phenyl-3H-imidazo[4,5-b]pyridin-3-yl)benzyl)carbamoyl)phenyl)-1H-pyrazole-4-carboxylic acid (I-11)

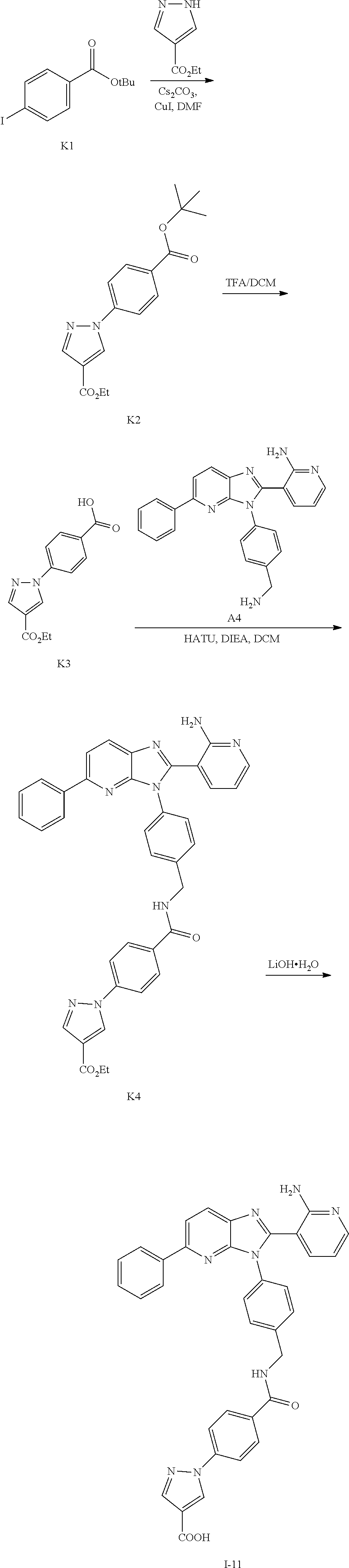

Step 1. To a solution of tert-butyl 4-iodobenzoate K1 (600 mg, 1.97 mmol) in DMF (8.00 mL) was added ethyl 1H-pyrazole-4-carboxylate (276 mg, 1.97 mmol), Cs₂CO₃ (1.28 g, 3.95 mmol) and CuI (38.0 mg, 0.200 mmol) at 25° C. The mixture was stirred at 100° C., for 16 hrs. Aq. LiCl (20 mL, 3%) was added and the resulting mixture was extracted with EtOAc (30 mL×3). The combined organic phase was washed with brine (10 mL), water (10 mL), dried over anhydrous Na₂SO₄, filtered and concentrated. The residue was purified by flash silica gel chromatography (eluent with 0-16% of EtOAc in PE) to give ethyl 1-(4-(tert-butoxycarbonyl) phenyl)-1H-pyrazole-4-carboxylate K2 (230 mg, 36.9% yield) as a white solid. 1H NMR (400 MHz, DMSO-d₆) δ_{H}=9.22 (s, 1H), 8.20 (s, 1H), 8.11-8.06 (m, 2H), 8.05-8.00 (m, 2H), 4.29 (q, J=6.8 Hz, 2H), 1.57 (s, 9H), 1.31 (t, J=7.2 Hz, 3H).

Step 2. To a solution of ethyl 1-(4-(tert-butoxycarbonyl)phenyl)-1H-pyrazole-4-carboxylate K2 (100 mg, 0.320 mmol) in DCM (1.00 mL), TFA (1.00 mL) was added at 25° C., and the mixture stirred for 2 hrs. The reaction mixture was concentrated to give 4-(4-(ethoxycarbonyl)-1H-pyrazol-1-yl)benzoic acid K3 (80.0 mg, 97.3% yield) as a colorless oil, which was used in the next step directly. LCMS Rₜ=0.518 min in 1.0 min chromatography, purity 84.9%, MS ESI calcd, for 260.08 [M+H]⁺ 261.08, found 261.0.

Step 3. To a solution of 4-(4-(ethoxycarbonyl)-1H-pyrazol-1-yl)benzoic acid K3 (80.0 mg, 0.310 mmol) in DCM (5.00 mL), DIEA (148 mg, 1.15 mmol), HATU (218 mg, 0.570 mmol) and 3-(3-(4-(aminomethyl)phenyl)-5-phenyl-3H-imidazo[4,5-b]pyridin-2-yl)pyridin-2-amine A4 (150 mg, 0.380 mmol) were added at 25° C., and the mixture stirred for 2 hrs. The reaction mixture was concentrated directly and the resultant residue was purified by flash silica gel chromatography (eluent with 0-6% of MeOH in DCM) to give 1-(4-((4-(2-(2-aminopyridin-3-yl)-5-phenyl-3H-imidazo[4,5-b]pyridin-3-yl)benzyl)carbamoyl)phenyl)-1H-pyrazole-4-carboxylate K4 (200 mg, 82.5% yield) as a yellow solid. ¹H NMR (400 MHz, DMSO-d₆)⁶H=9.26 (t, J=6.0 Hz, 1H), 9.22 (s, 1H), 8.20-8.16 (m, 2H), 8.10-7.98 (m, 10H), 7.51-7.38 (m, 7H), 7.23 (dd, J=7.6, 2.0 Hz, 1H), 6.43 (dd, J=7.6, 4.8 Hz, 1H), 4.63 (d, J=6.0 Hz, 2H), 4.29 (d, J=7.2 Hz, 2H), 1.31 (s, 3H).

Step 4. To a solution of 1-(4-((4-(2-(2-aminopyridin-3-yl)-5-phenyl-3H-imidazo[4,5-b]pyridin-3-yl)benzyl)carbamoyl)phenyl)-1H-pyrazole-4-carboxylate K4 (100 mg, 0.160 mmol) in THF (2.00 mL) and water (2.00 mL), LiOH·H₂O (13.0 mg, 0.320 mmol) was added at 25° C., and the mixture stirred for 2 hrs. The reaction mixture was concentrated to remove THF and the residue was purified by prep-HPLC (column: Xtimate C18 150*40 mm*10 μm; condition: water (HCl)-ACN; begin B: 10-50) to give 1-(4-((4-(2-(2-aminopyridin-3-yl)-5-phenyl-3H-imidazo[4,5-b]pyridin-3-yl)benzyl)carbamoyl)phenyl)-1H-pyrazole-4-carboxylic acid I-11 (11.0 mg, 11.4% yield) as a yellow solid. ¹H NMR (400 MHz, DMSO-d₆) δ_{H}=9.27 (t, J=6.0 Hz, 1H), 9.14 (s, 1H), 8.39-8.18 (m, 2H), 8.15-8.02 (m, 10H), 7.88-7.82 (m, 1H), 7.57-7.46 (m, 6H), 7.44-7.38 (m, 1H), 6.86 (dd, J=7.2, 6.4 Hz, 1H), 4.63 (d, J=6.0 Hz, 2H). HPLC Rₜ=4.345 min in 8 min chromatography, purity 96.4%. LCMS Rₜ=1.983 min in 4 min chromatography, purity 98.4%. MS ESI calcd, for 606.21 [M+H]⁺ 607.21, found 607.3.

### Example 12. Synthesis of N-(4-(2-(2-aminopyridin-3-yl)-5-phenyl-3H-imidazo[4,5-b]pyridin-3-yl)benzyl)-4-(4-cyano-1H-pyrazol-1-yl)benzamide (I-12)

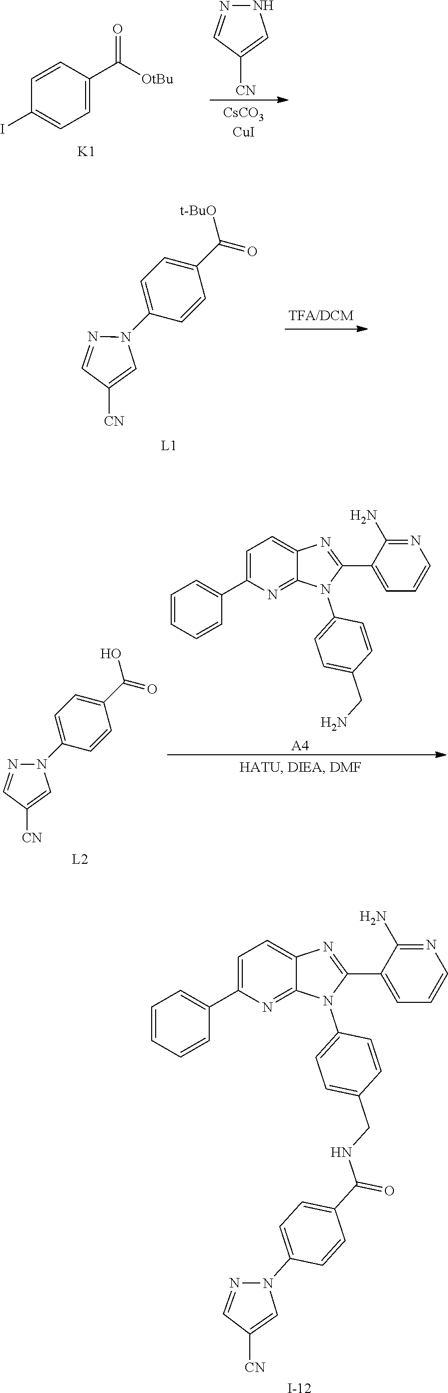

Step 1. To a solution of tert-butyl 4-iodobenzoate K1 (1.00 g, 3.29 mmol) in DMF (10.0 mL) was added 1H-pyrazole-4-carbonitrile (612 mg, 6.58 mmol), Cs₂CO₃ (2.14 g, 6.58 mmol) and CuI (63.0 mg, 0.330 mmol) at 25° C. The mixture was stirred at 100° C., for 24 hrs. Aq. LiCl (20 mL, 3%) was added and the resulting mixture was extracted with EtOAc (30 mL×3). The combined organic phase was washed with brine (10 mL), water (10 mL), dried over anhydrous Na₂SO₄, filtered and concentrated. The residue was purified by flash silica gel chromatography (eluent with 0-18% of EtOAc in PE) to give tert-butyl 4-(4-cyano-1H-pyrazol-1-yl)benzoate L1 (210 mg, 23.7% yield) as a gray solid. ¹H NMR (400 MHz, DMSO-d₆) δ_{H}=9.47 (s, 1H), 8.42 (s, 1H), 8.08-8.04 (m, 2H), 8.03-7.98 (m, 2H), 1.57 (s, 9H).

Step 2. To a solution of tert-butyl 4-(4-cyano-1H-pyrazol-1-yl)benzoate L1 (100 mg, 0.370 mmol) in DCM (1.00 mL), TFA (1.00 mL) was added at 25° C., and the mixture stirred for 2 hrs. The reaction mixture was concentrated to give 4-(4-cyano-1H-pyrazol-1-yl)benzoic acid L2 (79.0 mg, 99.8% yield) as a white solid, which was used in the next step directly. ¹H NMR (400 MHz, DMSO-d₆)⁶H=13.18 (s, 1H), 9.46 (s, 1H), 8.42 (s, 1H), 8.13-8.08 (m, 2H), 8.03-7.98 (m, 2H).

Step 3. To a solution of 4-(4-cyano-1H-pyrazol-1-yl)benzoic acid L2 (79.0 mg, 0.370 mmol) in DMF (2.00 mL). DIEA (144 mg, 1.11 mmol). HATU (211 mg, 0.560 mmol) and 3-(3-(4-(aminomethyl)phenyl)-5-phenyl-3H-imidazo[4,5-b]pyridin-2-yl)pyridin-2-amine A4 (145 mg, 0.370 mmol) were added at 25° C., and the mixture stirred for 2 hrs. Aq. LiCl (10 mL, 3%) was added and the resulting mixture was extracted with EtOAc (20 mL×3). The combined organic phase was washed with brine (5 mL), water (5 mL), dried over anhydrous Na₂SO₄, filtered and concentrated. The residue was purified by prep-HPLC (column: Xtimate C18 150*40 mm*10 μm; condition: condition: water (HCl)-ACN; begin B: 14-54%) to give N-(4-(2-(2-aminopyridin-3-yl)-5-phenyl-3H-imidazo[4,5-b]pyridin-3-yl)benzyl)-4-(4-cyano-1H-pyrazol-1-yl)benzamide I-12 (30.0 mg, 13.9% yield) as a yellow solid. ¹H NMR (400 MHz, DMSO-d₆) δ_{H}=9.46 (s, 1H), 9.31 (t, J=6.0 Hz, 1H), 8.42 (s, 1H), 8.38-8.19 (m, 2H), 8.16-8.08 (m, 3H), 8.07-7.98 (m, 5H), 7.85 (dd, J=7.6, 1.2 Hz, 1H), 7.58-7.38 (m, 7H), 6.86 (dd, J=7.2, 6.4 Hz, 1H), 4.63 (d, J=6.0 Hz, 2H). HPLC Rₜ=4.501 min in 8 min chromatography, purity 99.8%. LCMS Rₜ=1.272 min in 2 min chromatography, purity 96.3%, MS ESI calcd, for 587.22 [M+H]⁺ 588.22, found 588.0.

### Example 13. Synthesis of N-(4-(2-(2-aminopyridin-3-yl)-5-phenyl-3H-imidazo[4,5-b]pyridin-3-yl)benzyl)-4-(5-cyano-3-methyl-1H-pyrazol-1-yl)benzamide (I-13)

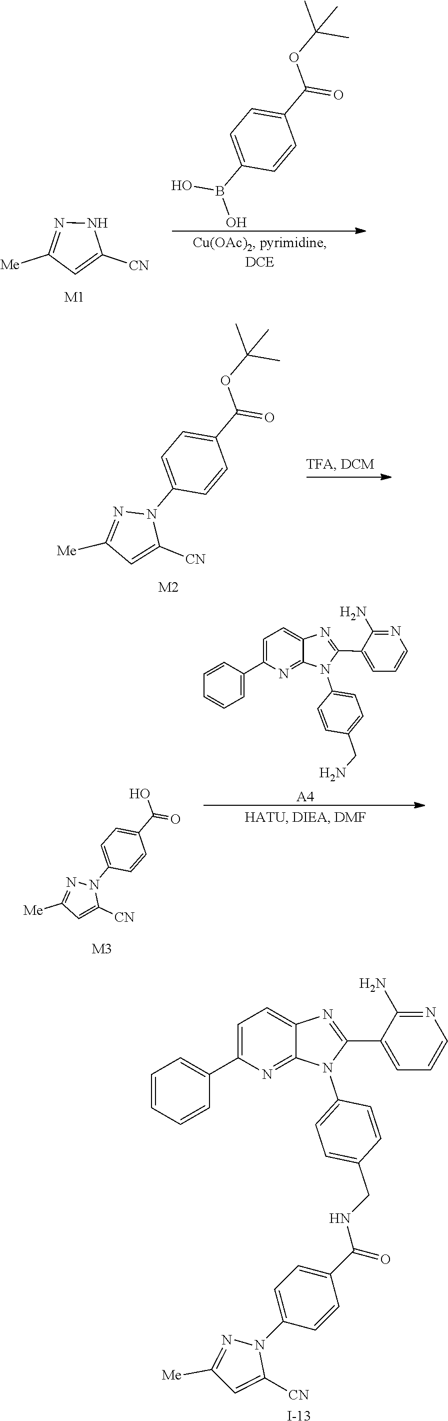

Step 1. A solution of 3-methyl-1H-pyrazole-5-carbonitrile M1 (500 mg, 4.67 mmol), (4-tert-butoxycarbonylphenyl)boronic acid (2.07 g, 9.34 mmol), Cu(OAc)₂ (1.69 g, 9.34 mmol) and pyridine (1.84 g, 23.3 mmol) in DCE (5.00 mL) was stirred at 60° C., for 12 hrs. The reaction mixture was filtered and the filter cake dissolved in water (100 mL). The resulting suspension was extracted with EtOAc (100 mL×3) and the combined organic phase was washed with water (100 mL), dried over anhydrous Na₂SO₄, filtered and concentrated. The residue was purified by flash silica gel chromatography (ISCO®; 12 g SepaFlash® Silica Flash Column, Eluent with 12% of Ethyl acetate in Petroleum ether gradient (@40 mL/min) to give tert-butyl 4-(5-cyano-3-methyl-1H-pyrazol-1-yl)benzoate M2 (158 mg, 12.0% yield) as colorless liquid. ¹H NMR (DMSO-d₆, 400 MHz) δ=8.09 (d, J=8.4 Hz, 2H), 7.85 (d, J=8.8 Hz, 2H), 7.34 (s, 1H), 2.34 (s, 3H), 1.57 (s, 9H). LCMS Rₜ=1.760 min in 2.5 min chromatography, purity 56.7%, MS ESI calcd, for 283.13 [M+H]⁺ 284.13, found 284.2.

Step 2. To a solution of tert-butyl 4-(5-cyano-3-methyl-1H-pyrazol-1-yl)benzoate M2 (158 mg, 0.550 mmol) in DCM (5.00 mL), TFA (2.00 mL) was added at 25° C., and the mixture stirred for 12 hrs. The reaction mixture was concentrated directly to give 4-(5-cyano-3-methyl-1H-pyrazol-1-yl)benzoic acid M3 (161 mg, crude) as a white solid. The crude product was used in the next step without further purification. ¹H NMR (DMSO-d₆, 400 MHz) δ=8.13 (d, J=8.8 Hz, 2H), 7.85 (d, J=8.8 Hz, 2H), 7.33 (s, 1H), 2.49 (s, 3H). LCMS Rₜ=1.156 min in 2.5 min chromatography, purity 85.8%, MS ESI calcd, for 227.07 [M+H]⁺ 228.07, found 228.1.

Step 3. To a solution of 3-(3-(4-(aminomethyl)phenyl)-5-phenyl-3H-imidazo[4,5-b]pyridin-2-yl)pyridin-2-amine A4 (60.0 mg, 0.150 mmol) in DMF (1.00 mL), 4-(5-cyano-3-methyl-1H-pyrazol-1-yl)benzoic acid M3 (35.0 mg, 0.150 mmol) and DIEA (99.0 mg, 0.760 mmol) were added. After addition, the mixture was stirred at 25° C., for 5 mins, and then HATU (87 mg, 0.22 mmol) was added. The resulting mixture was stirred at 25° C., for a further 2 hrs. The crude product was purified by prep-HPLC (column: Xtimate C18 150*40 mm*10 μm; mobile phase: water (FA)-ACN; B %: 16%-56%, 25 min) to give N-(4-(2-(2-aminopyridin-3-yl)-5-phenyl-3H-imidazo[4,5-b]pyridin-3-yl)benzyl)-4-(5-cyano-3-methyl-1H-pyrazol-1-yl)benzamide I-13 (17.5 mg, 18.6% yield) as a white solid. ¹H NMR (DMSO-d₆, 400 MHz) δ=9.32 (d, J=6.0 Hz, 1H), 8.26 (d, J=8.4 Hz, 1H), 8.13 (d, J=8.4 Hz, 2H), 8.06-7.95 (m, 4H), 7.84 (d, J=8.8 Hz, 2H), 7.55-7.42 (m, 6H), 7.42-7.35 (m, 1H), 7.32 (s, 1H), 7.22 (dd, J=7.6, 1.6 Hz, 1H), 6.94 (s, 2H), 6.42 (dd, J=7.6, 4.8 Hz, 1H), 4.63 (d, J=5.2 Hz, 2H), 2.34 (s, 3H). HPLC Rₜ=4.855 min in 8 min chromatography, purity 97.5%. LCMS Rₜ=2.766 min in 4 min chromatography, purity 95.6%, MS ESI calcd, for 601.23 [M+H]⁺ 602.23, found 602.3.

### Example 14. Synthesis of N-(4-(2-(2-aminopyridin-3-yl)-5-phenyl-3H-imidazo[4,5-b]pyridin-3-yl)benzyl)-4-(5-cyano-3-methyl-1H-pyrazol-1-yl)benzamide (I-14)

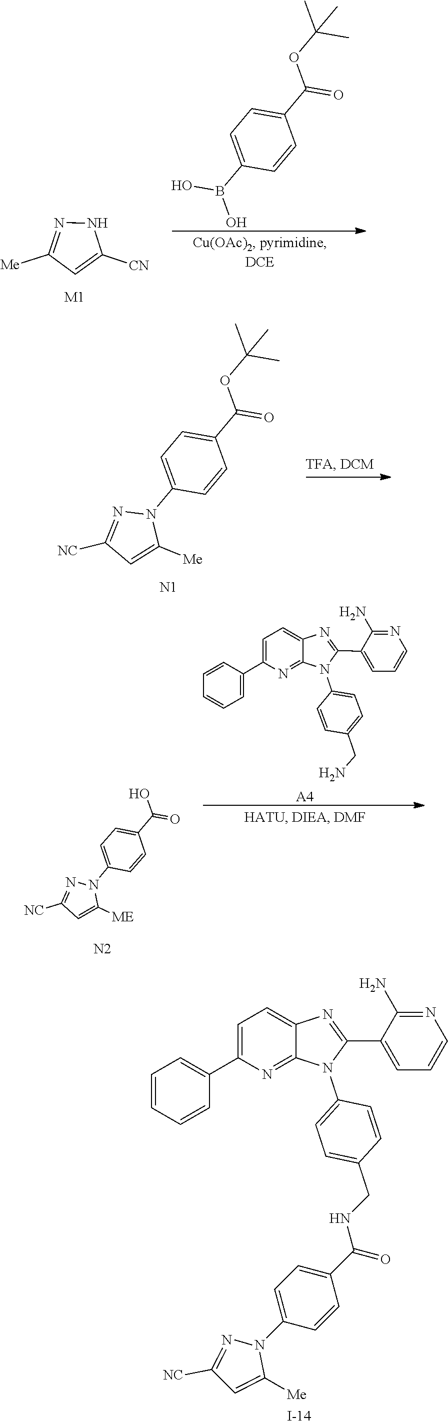

Step 1. A solution of 3-methyl-1H-pyrazole-5-carbonitrile M1 (500 mg, 4.67 mmol), (4-tert-butoxycarbonylphenyl)boronic acid (2.07 g, 9.34 mmol), Cu(OAc)₂ (1.69 g, 9.34 mmol) and pyridine (1.84 g, 23.3 mmol) in DCE (5.00 mL) was stirred at 60° C., for 12 hrs. The reaction mixture was filtered and the filter cake dissolved in water (100 mL). The resulting suspension was extracted with EtOAc (100 mL×3). The combined organic phase was washed with water (100 mL), dried over anhydrous Na₂SO₄, filtered and concentrated. The residue was purified by flash silica gel chromatography (ISCO®; 12 g SepaFlash@Silica Flash Column, Eluent with 12% of Ethyl acetate in Petroleum ether gradient @40 mL/min) and then purified by prep-HPLC (column: Xtimate C18 150*40 mm*10 μm; mobile phase: water (NH₄HCO₃)-ACN; B %: 40%-80%, 32 min) to give tert-butyl 4-(3-cyano-5-methyl-1H-pyrazol-1-yl)benzoate N1 (192 mg, 14.5% yield) as a white solid. 1H NMR (DMSO-d₆, 400 MHz) δ_{H}=8.07 (d, J=8.4 Hz, 2H), 7.74 (d, J=8.8 Hz, 2H), 7.02 (s, 1H), 2.38 (s, 3H), 1.57 (s, 9H).

Step 2. To a solution of tert-butyl 4-(3-cyano-5-methyl-1H-pyrazol-1-yl)benzoate N1 (50.0 mg, 0.17 mmol) in DCM (2.00 mL). TFA (2.00 mL) was added at 25° C., and the mixture stirred for 12 hrs. The reaction mixture was concentrated directly to give 4-(3-cyano-5-methyl-1H-pyrazol-1-yl)benzoic acid N2 (47.0 mg, crude) as a white solid, which was used in the next step without further purification. ¹H NMR (DMSO-d₆, 400 MHz) δ_{H}=8.11 (d, J=8.8 Hz, 2H), 7.74 (d, J=8.8 Hz, 2H), 7.03 (s, 1H), 2.39 (s, 3H).

Step 3. To a solution of 3-(3-(4-(aminomethyl)phenyl)-5-phenyl-3H-imidazo[4,5-b]pyridin-2-yl)pyridin-2-amine A4 (80.0 mg, 0.200 mmol) in DMF (3.00 mL), 4-(3-cyano-5-methyl-1H-pyrazol-1-yl)benzoic acid N2 (47.0 mg, 0.200 mmol) and DIEA (79.0 mg, 0.610 mmol) were added. After addition, the mixture was stirred at 25° C., for 5 mins, and then HATU (117 mg, 0.300 mmol) was added. The resulting mixture was stirred at 25° C., for 2 hrs. The mixture was diluted with H₂O (10 mL), extracted with EtOAc (10 mL×3), washed with brine (30 mL) and the organic layer was dried over Na₂SO₄, filtered and concentrated. The crude product was purified by prep-HPLC (column: Xtimate C18 150*40 mm*10 μm; mobile phase: water (FA)-ACN; B %: 16%-56%, 25 min) to give N-(4-(2-(2-aminopyridin-3-yl)-5-phenyl-3H-imidazo[4,5-b]pyridin-3-yl)benzyl)-4-(5-cyano-3-methyl-1H-pyrazol-1-yl)benzamide I-14 (27.4 mg, 21.1% yield) as a white solid. ¹H NMR (DMSO-d₆, 400 MHz) δ_{H}=9.33 (d, J=6.0 Hz, 1H), 8.26 (d, J=7.6 Hz, 1H), 8.12 (d, J=8.8 Hz, 2H), 8.06-7.96 (m, 4H), 7.74 (d, J=8.4 Hz, 2H), 7.54-7.43 (m, 6H), 7.43-7.36 (m, 1H), 7.24 (dd, J=7.6, 2.0 Hz, 1H), 7.02 (s, 1H), 6.98 (s, 2H), 6.43 (dd, J=7.6, 4.8 Hz, 1H), 4.64 (d, J=5.6 Hz, 2H), 2.39 (s, 3H). HPLC Rₜ=4.747 min in 8 min chromatography, purity 94.8%. LCMS Rₜ=2.695 min in 4 min chromatography, purity 93.6%, MS ESI calcd, for 601.23 [M+H]⁺ 602.23, found 602.2.

### Example 15. Synthesis of N-(4-(2-(2-aminopyridin-3-yl)-5-phenyl-3H-imidazo[4,5-b]pyridin-3-yl)benzyl)-4-(3-cyano-5-hydroxy-1H-pyrazol-1-yl)benzamide (I-15)

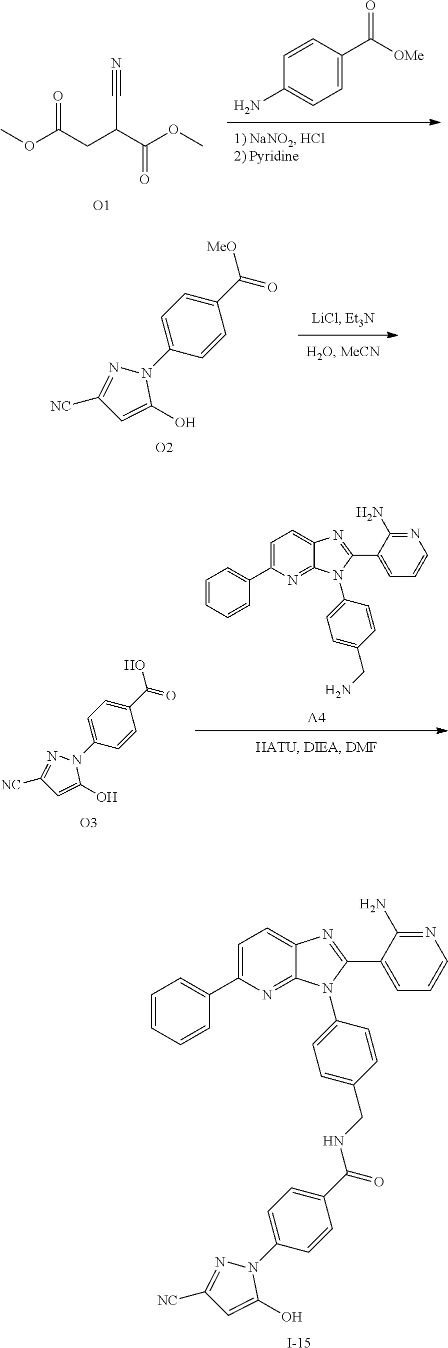

Step 1. To a mixture of water (5.86 mL) and HCl (0.720 mL) was added dimethyl 2-cyanosuccinate 01 (500 mg, 2.92 mmol). A solution of NaNO₂ (202 mg, 2.92 mmol) in water (1.17 mL) was added slowly at 0° C. After addition, the mixture was stirred at 0° C., for 1 hr. The solution was added to methyl 4-aminobenzoate (442 mg, 2.92 mmol) in pyridine (5.86 mL) dropwise at 0° C. The resulting mixture was stirred at 0° C., for 1 hr and 20° C., for 1 hr, 2% aqueous NaOH (12 mL) was added to the reaction mixture. The resulting mixture was stirred 20° C., for 2 hrs. 35% HCl (12 mL) was added to the reaction mixture under ice-cooling. The reaction mixture was filtered and the filter cake was concentrated to dryness. The residue was purified by combi-flash column (EtOAc in PE=0%-23%) to give methyl 4-(3-cyano-5-hydroxy-1H-pyrazol-1-yl)benzoate 02 (500 mg, 70.4% yield) as colorless oil. ¹H NMR (DMSO-d₆, 400 MHz)^{6H}=8.13-8.08 (m, 2H), 7.98-7.92 (m, 2H), 6.23 (s, 1H), 3.88 (s, 3H).

Step 2. To a solution of methyl 4-(3-cyano-5-hydroxy-1H-pyrazol-1-yl)benzoate 02 (400 mg, 1.64 mmol) in MeCN (1.00 mL), methanol (1.50 mL), THF (1.50 mL), water (1.50 mL), LiCl (0.700 mg, 0.0164 mmol), TEA (1.10 mL, 8.22 mmol), LiOH·H₂O (207 mg, 4.93 mmol) were added at 20° C., and the mixture was stirred at 50° C., for 2 hrs. The reaction mixture was subsequently concentrated to remove THF and MeOH, and the resulting mixture was extracted with PE (10 mL×3). The combined water phase was then adjusted to pH~5-6 by aq. HCl (1M). The reaction mixture was filtered and the filter cake was concentrated to give 4-(3-cyano-5-hydroxy-1H-pyrazol-1-yl)benzoic acid 03 (400 mg, crude) as a yellow solid, which was used to the next step without purification. LCMS Rₜ=1.094 min in 2.5 min chromatography, purity 61.5%, MS ESI calcd, for 229.05, [M+H]⁺ 230.05, found 230.0.

Step 3. To a solution of 3-(3-(4-(aminomethyl)phenyl)-5-phenyl-3H-imidazo[4,5-b]pyridin-2-yl)pyridin-2-amine A4 (100 mg, 0.250 mmol) in DMF (1.00 mL), 4-(3-cyano-5-hydroxy-1H-pyrazol-1-yl)benzoic acid 03 (58.0 mg, 0.250 mmol), DIEA (0.160 g, 1.27 mmol) and HATU (0.190 g, 0.510 mmol) were added and the resulting mixture was stirred at 25° C., for 2 hrs. The reaction mixture was concentrated directly and the residue was purified by prep-HPLC (condition: water (FA)-ACN; Column: Xtimate C18 150*40 mm*10 μm; B %: 12%-52%; Gradient Time, 25 min) to give N-(4-(2-(2-aminopyridin-3-yl)-5-phenyl-3H-imidazo[4,5-b]pyridin-3-yl)benzyl)-4-(3-cyano-5-hydroxy-1H-pyrazol-1-yl)benzamide I-15 (20.1 mg, 13.1% yield) as a white solid. ¹H NMR (DMSO-d₆, 400 MHz) δ_{H}=9.26 (s, 1H), 8.27 (d, J=8.0 Hz, 1H), 8.07 (d, J=8.8 Hz, 2H), 8.05-7.97 (m, 4H), 7.88 (d, J=8.8 Hz, 2H), 7.54-7.42 (m, 6H), 7.41 (s, 1H), 7.25 (d, J=8.0 Hz, 1H), 7.01 (s, 2H), 6.48-6.43 (m, 1H), 6.21 (s, 1H), 4.63 (d, J=6.0 Hz, 2H). HPLC Rₜ=3.046 min in 8 min chromatography, purity 97.7%. LCMS Rₜ=1.810 min in 4 min chromatography, purity 97.7%, MS ESI calcd, for 603.21, [M+H]⁺ 604.21, found 604.2.

### Example 16. Synthesis of 1-(4-((4-(2-(2-aminopyridin-3-yl)-5-phenyl-3H-imidazo[4,5-b]pyridin-3-yl)benzyl)carbamoyl)phenyl)-3-methyl-1H-pyrazole-5-carboxamide (I-16)

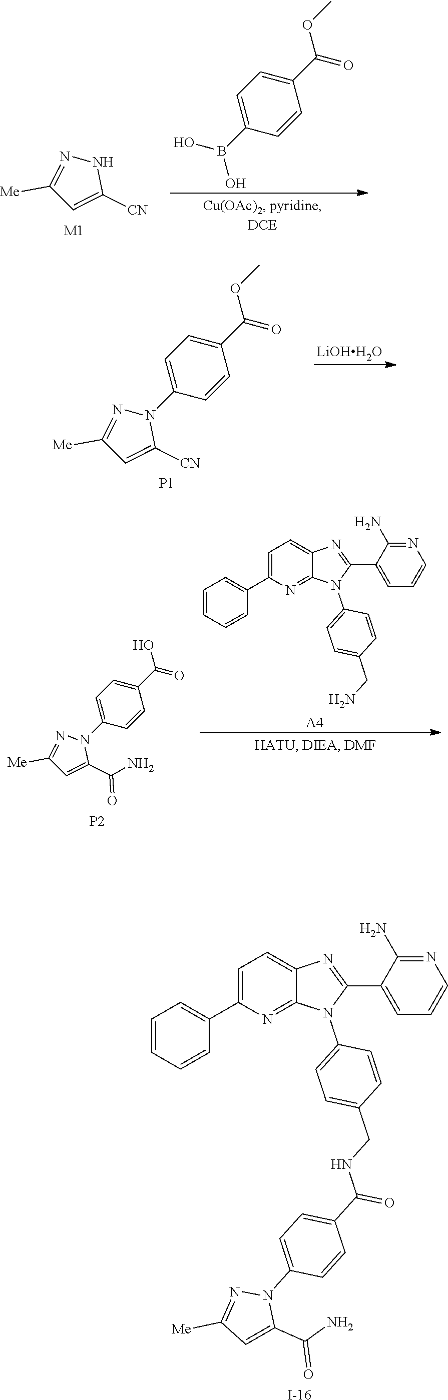

Step 1. A mixture of 3-methyl-1H-pyrazole-5-carbonitrile M1 (1.00 g, 9.34 mmol), (4-methoxycarbonylphenyl)boronic acid (3.36 g, 18.7 mmol). Cu(OAc)₂ (3.39 g, 18.7 mmol) and pyridine (3.68 g, 46.7 mmol) in DCE (15.0 mL) was stirred at 60° C., for 12 hrs. The reaction mixture was filtered and the residue dissolved in water (100 mL). The resulting suspension was extracted with EtOAc (50 mL×3), and the combined organic phase was dried over anhydrous Na₂SO₄, filtered and concentrated. The residue was purified by flash silica gel chromatography (ISCO®; 20 g SepaFlashX Silica Flash Column, Eluent of 15% Ethyl acetate/Petroleum ether gradient @50 mL/min) and then purified by prep-HPLC (column: Xtimate C18 150*40 mm*10 μm; mobile phase: water (NH₄HCO₃)-ACN; B %: 28%-68%, 32 min) to give methyl 4-(5-cyano-3-methyl-1H-pyrazol-1-yl)benzoate P1 (330 mg, 14.7% yield) as a white solid. ¹H NMR (DMSO-d₆, 400 MHz) δ=8.15 (d, J=8.8 Hz, 2H), 7.88 (d, J=8.8 Hz, 2H), 7.34 (s, 1H), 3.89 (s, 3H), 2.33 (s, 3H). LCMS Rₜ=2.154 min in 4 min chromatography, purity 99.4%, MS ESI calcd, for 241.09 [M+H]⁺ 242.09, found 242.2.

Step 2. To a solution of methyl 4-(5-cyano-3-methyl-1H-pyrazol-1-yl)benzoate P1 (150 mg, 0.620 mmol) in methanol (3.00 mL) was added hydroxylithium;hydrate (130 mg, 3.11 mmol), THF (2.00 mL) and water (2.00 mL). The resulting mixture was stirred at 50° C. for 2 hrs. The reaction mixture was concentrated, diluted with H₂O (10 mL) and extracted with PE (10 mL×3). The pH of the aqueous phase was adjusted to pH~5 with HCl (2M). The resulting mixture was extracted with DCM (10 mL×3). The combined organic phase was dried over anhydrous Na₂SO₄, filtered and concentrated to give 4-(5-carbamoyl-3-methyl-1H-pyrazol-1-yl)benzoic acid P2 (149 mg, 97.5% yield) as a white solid, which was used in the next step without further purification. LCMS Rₜ=0.860 min in 2.5 min chromatography, purity 49.0%, MS ESI calcd, for 245.08 [M+H]⁺ 246.08, found 246.1.

Step 3. To a solution of 3-(3-(4-(aminomethyl)phenyl)-5-phenyl-3H-imidazo[4,5-b]pyridin-2-yl)pyridin-2-amine A4 (238 mg, 0.600 mmol) in DMF (3.00 mL), 4-(5-carbamoyl-3-methyl-1H-pyrazol-1-yl)benzoic acid P2 (149 mg, 0.600 mmol) and DIEA (235 mg, 1.82 mmol) were added. After addition, the mixture was stirred at 25° C., for 5 mins, and then HATU (346 mg, 0.90 mmol) was added. The resulting mixture was stirred at 25° C., for an additional 2 hrs. The mixture was diluted with H₂O (10 mL), extracted with EtOAc (10 mL×3), washed with brine (10 mL) and the organic layer dried over Na₂SO₄, filtered and concentrated. The crude product was purified by prep-HPLC (column: Xtimate C18 150*40 mm*10 μm; mobile phase: water (NH₄HCO₃)-ACN; B %: 18%-58%, 32 min) to give 1-(4-((4-(2-(2-aminopyridin-3-yl)-5-phenyl-3H-imidazo[4,5-b]pyridin-3-yl)benzyl)carbamoyl)phenyl)-3-methyl-1H-pyrazole-5-carboxamide I-16 (38.5 mg, 10.1% yield) as a white solid. ¹H NMR (DMSO-d₆, 400 MHz) δ=9.23 (d, J=6.0 Hz, 1H), 8.26 (d, J=8.4 Hz, 1H), 8.06-7.96 (m, 7H), 7.58 (s, 1H), 7.52-7.43 (m, 8H), 7.42-7.35 (m, 1H), 7.22 (dd, J=7.6, 2.0 Hz, 1H), 6.94 (s, 2H), 6.71 (s, 1H), 6.42 (dd, J=7.6, 4.8 Hz, 1H), 4.63 (d, J=6.0 Hz, 2H), 2.25 (s, 3H). HPLC Rₜ=3.791 min in 8 min chromatography, purity 98.6%. LCMS Rₜ=2.178 min in 4 min chromatography, purity 97.3%, MS ESI calcd, for 619.24 [M+H]⁺ 620.24, found 620.3.

### Example 17. Synthesis of 1-(4-((4-(2-(2-aminopyridin-3-yl)-5-phenyl-3H-imidazo[4,5-b]pyridin-3-yl)benzyl)carbamoyl)phenyl)-5-methyl-1H-pyrazole-3-carboxamide (I-17)

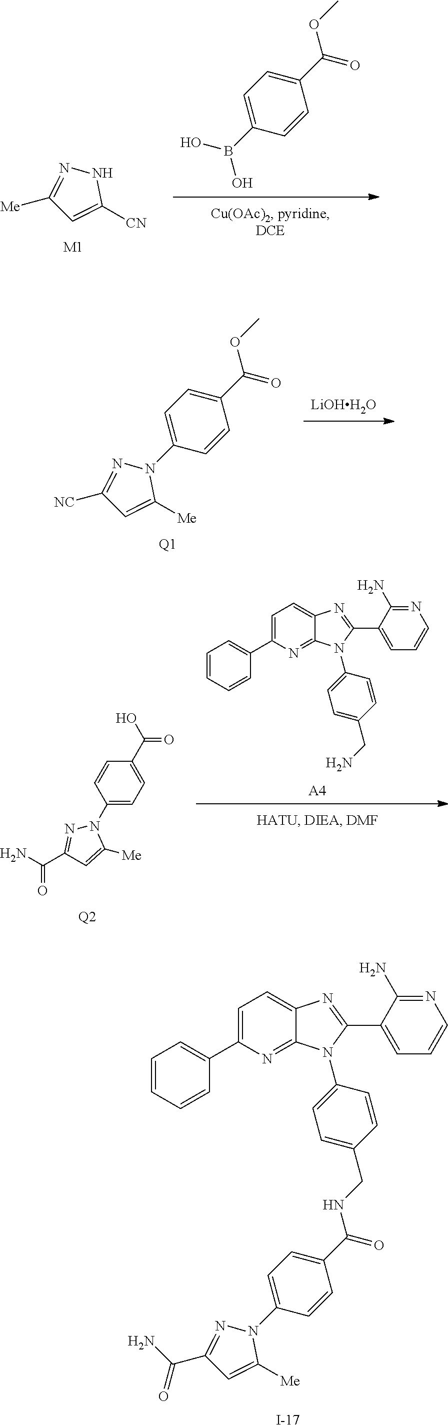

Step 1. A mixture of 3-methyl-1H-pyrazole-5-carbonitrile M1 (500 mg, 4.67 mmol), (4-methoxycarbonylphenyl)boronic acid (1.68 g, 9.34 mmol). Cu(OAc)₂ (1.69 g, 9.34 mmol), and pyridine (1.84 g, 23.3 mmol) in DCE (10.0 mL) was stirred at 60° C., for 16 hrs. The reaction mixture was filtered and the filtrate was concentrated. The residue was dissolved in water (10 mL) and the resulting suspension was extracted with EtOAc (20 mL×3). The combined organic phase was washed with brine (20 mL), dried over anhydrous Na₂SO₄, filtered and concentrated. The residue was purified by flash silica gel chromatography (ISCO®; 12 g SepaFlash® Silica Flash Column, Eluent of 13% Ethyl acetate/Petroleum ether gradient @35 mL/min) to give methyl 4-(3-cyano-5-methyl-1H-pyrazol-1-yl)benzoate Q1 (407 mg, 36.1% yield) as a white solid. ¹H NMR (DMSO-d₆. 400 MHz) δ=8.14 (d, J=8.8 Hz, 2H), 7.79 (d, J=8.8 Hz, 2H), 7.03 (s, 1H), 3.90 (s, 3H), 2.40 (s, 3H).

Step 2. To a solution of methyl 4-(3-cyano-5-methyl-1H-pyrazol-1-yl)benzoate Q1 (407 mg, 1.69 mmol) in methanol (5.00 mL), hydroxylithium;hydrate (354 mg, 8.43 mmol), THF (3.00 mL) and water (3.00 mL) were added. The resulting mixture was stirred at 25° C. for 2 hrs and the reaction mixture was concentrated to give the crude product. The mixture was diluted with H₂O (10 mL) and extracted with PE (10 mL×3). The pH of the aqueous phase was adjusted to pH~4 with HCl (2M). The resulting mixture was extracted with DCM (10 mL×3). The combined organic phase was dried over anhydrous Na₂SO₄, filtered and concentrated to give 4-(3-carbamoyl-5-methyl-1H-pyrazol-1-yl)benzoic acid Q2 (269 mg, 64.9% yield) as a white solid, which was used in the next step without further purification. LCMS Rₜ=0.884 min in 2.5 min chromatography, purity 53.7%. MS ESI calcd, for 245.08 [M+H]⁺ 246.08, found 246.1.

Step 3. To a solution of 3-(3-(4-(aminomethyl)phenyl)-5-phenyl-3H-imidazo[4,5-b]pyridin-2-yl)pyridin-2-amine A4 (258 mg, 0.650 mmol) in DMF (4.00 mL), 4-(3-carbamoyl-5-methyl-1H-pyrazol-1-yl)benzoic acid Q2 (269 mg, 1.10 mmol), DIEA (425 mg, 3.29 mmol) and then HATU (375 mg, 0.980 mmol) were added at 25° C., and the reaction mixture stirred for 3 hrs. The mixture was diluted with H₂O (10 mL), extracted with EtOAc (10 mL×3), washed with brine (20 mL). The organic layer was dried over Na₂SO₄, concentrated and the crude product was purified by prep-HPLC (column: Xtimate C18 150*40 mm*10 μm; mobile phase: [water (HCl)-ACN]; B %: 10%-50%, 36 min) to give 1-(4-((4-(2-(2-aminopyridin-3-yl)-5-phenyl-3H-imidazo[4,5-b]pyridin-3-yl)benzyl)carbamoyl)phenyl)-5-methyl-1H-pyrazole-3-carboxamide I-17 (19.9 mg, 4.76% yield) as a white solid. ¹H NMR (DMSO-d₆, 400 MHz) δ=9.34 (d, J=6.0 Hz, 1H), 8.35 (d, J=8.4 Hz, 1H), 8.15-8.09 (m, 3H), 8.09-8.01 (m, 3H), 7.89 (d, J=7.6 Hz, 1H), 7.74 (d, J=8.4 Hz, 2H), 7.61-7.38 (m, 8H), 7.30 (s, 1H), 6.89 (d, J=6.4 Hz, 1H), 6.66 (s, 1H), 4.63 (d, J=5.6 Hz, 2H), 2.39 (s, 3H). HPLC Rₜ=4.131 min in 8 min chromatography, purity 97.4%. LCMS Rₜ=2.331 min in 4 min chromatography, purity 98.0%. MS ESI calcd, for 619.24 [M+H]⁺ 620.24, found 620.3.

### Example 18. Synthesis of N-(4-(2-(2-aminopyridin-3-yl)-5-phenyl-3H-imidazo[4,5-b]pyridin-3-yl)benzyl)-6-(5-hydroxy-3-methyl-1H-pyrazol-1-yl)nicotinamide (I-18)

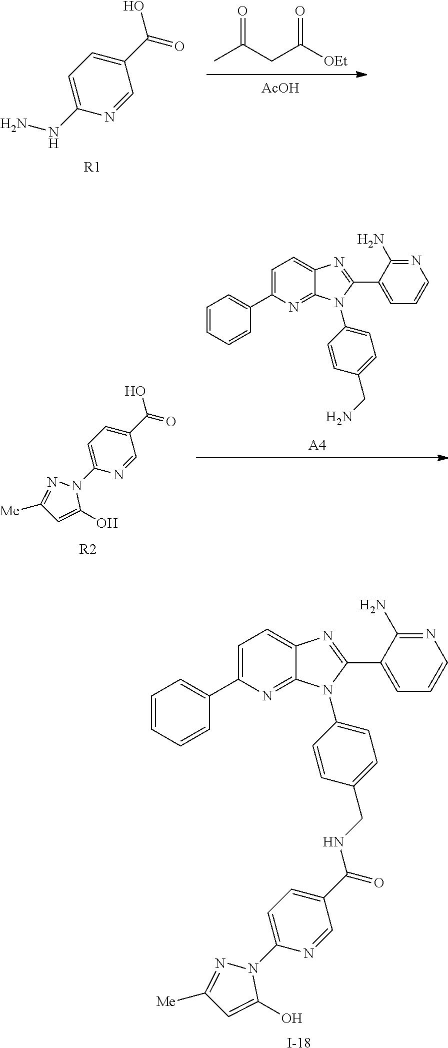

Step 1. To a mixture of 6-hydrazineylnicotinic acid RI (200 mg, 1.31 mmol) in acetic acid (4.00 mL), ethyl 3-oxobutanoate (170 mg, 1.31 mmol) was added. The mixture was stirred at 90° C., for 2 hrs. The mixture was directly concentrated to give 6-(5-hydroxy-3-methyl-TH-pyrazol-1-yl)nicotinic acid R² (300 mg, 87.6% yield) as a white solid. ¹H NMR (400 MHz, DMSO-d₆) δ_{H}=13.00-12.42 (m, 1H), 12.22 (s, 1H), 8.91-8.86 (m, 1H), 8.60-8.46 (m, 1H), 8.40-8.28 (m, 1H), 5.25-5.00 (m, 1H), 1.91 (s, 3H).

Step 2. To a solution of 3-(3-(4-(aminomethyl)phenyl)-5-phenyl-3H-imidazo[4,5-b]pyridin-2-yl)pyridin-2-amine A4 (500 mg, 1.26 mmol) in pyridine (4.00 mL), 6-(5-hydroxy-3-methyl-1H-pyrazol-1-yl)nicotinic acid R² (246 mg, 1.26 mmol) and EDCI (73.0 mg, 0.380 mmol) were added, and the mixture stirred at 25° C., for 2 hrs. The reaction mixture was diluted with water (20 mL) and extracted with EtOAc (30 mL×2). The combined organic phase was washed with brine (30 mL×2), dried over anhydrous Na₂SO₄, filtered and concentrated. The residue was purified by prep-HPLC (Column: Welch Xtimate C18 150*40 mm*10 μm; mobile phase: [water (HCl)-ACN]; B %: 8%-48%; 36 min) to give N-(4-(2-(2-aminopyridin-3-yl)-5-phenyl-3H-imidazo[4,5-b]pyridin-3-yl)benzyl)-6-(5-hydroxy-3-methyl-1H-pyrazol-1-yl)nicotinamide I-18 (6.30 mg, 4.0% yield) as a yellow solid. ¹H NMR (400 MHz, DMSO-d₆) δ_{H}=9.40 (t, J=1.6 Hz, 1H), 8.97 (d, J=1.6 Hz, 1H), 8.52-8.40 (m, 2H), 8.39-8.34 (m, 2H), 8.13 (dd, J=1.6, 6.4 Hz, 1H), 8.07-8.02 (m, 3H), 7.91 (dd, J=1.6, 7.2 Hz, 1H), 7.59-7.51 (m, 4H), 7.51-7.46 (m, 2H), 7.44-7.38 (m, 1H), 6.93-6.87 (m, 1H), 5.22 (s, 1H), 4.63 (d, J=5.6 Hz, 2H), 2.19 (s, 3H). HPLC Rₜ=2.854 min in 8 min chromatography, purity 97.0%. LCMS Rₜ=1.724 min in 4 min; Agilent PoroShell 120 EC-C18 2.7 μm 3.0*50 mm; purity 98.5%. MS ESI calcd, for 593.23. [M+H]⁺ 594.23, found 594.3.

### Example 19. Synthesis of N-(4-(2-(2-aminopyridin-3-yl)-5-phenyl-3H-imidazo[4,5-b]pyridin-3-yl)benzyl)-5-(5-hydroxy-3-methyl-1H-pyrazol-1-yl)picolinamide (I-19)

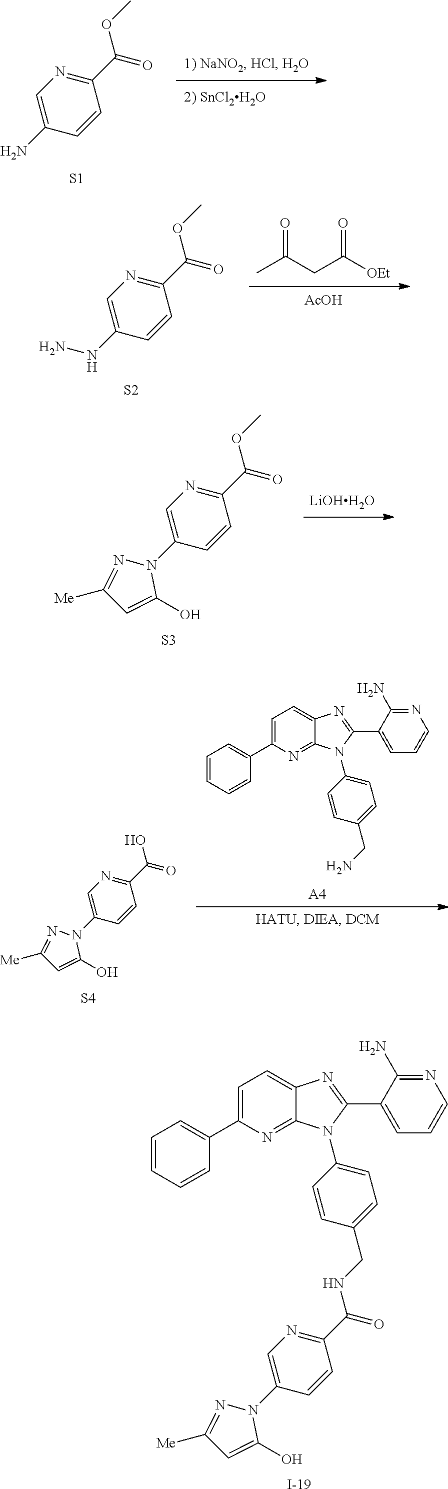

Step 1. A solution of methyl 5-aminopicolinate S1 (1.00 g, 6.58 mmol) in hydrochloric acid (10.0 mL, 12M) was cooled to 0° C. A solution of sodium nitrite (680 mg, 9.86 mmol) in water (10.0 mL) was added dropwise and the reaction mixture stirred at 0° C. for 20 mins. Tin(II) chloride dihydrate (2.97 g, 13.1 mmol) was added to the reaction mixture over 1 hr and the mixture stirred at 0° C., for 1 hr. The reaction mixture was quenched with 5M sodium hydroxide solution and adjusted to pH~8 by aq. NaOH. The resulting mixture was extracted with DCM (30 mL×3), and the combined organic phase washed with brine (10 mL), water (10 mL), dried over anhydrous Na₂SO₄, filtered and concentrated to give methyl 5-hydrazineylpicolinate S2 (300 mg, 27.3% yield) as a yellow solid. ¹H NMR (400 MHz, DMSO-d₆) δ=8.10 (d, J=2.4 Hz, 1H), 7.84-7.79 (m, 2H), 7.11 (dd, J=8.8, 2.8 Hz, 1H), 4.32 (d, J=2.0 Hz, 2H), 3.76 (s, 3H).

Step 2. To a solution of methyl 5-hydrazineylpicolinate S2 (250 mg, 1.50 mmol) in ethanol (3.00 mL) and acetic acid (3.00 mL), ethyl 3-oxobutanoate (0.190 mL, 1.50 mmol) was added at 25° C. The mixture was then allowed to stir at 90° C., for 16 hrs. The reaction mixture was concentrated directly and the residue was purified by flash silica gel chromatography (eluent with 0-10% of MeOH in DCM) to give methyl 5-(5-hydroxy-3-methyl-1H-pyrazol-1-yl)picolinate S3 (200 mg, 57.3% yield) as a yellow solid. ¹H NMR (400 MHz, DMSO-d₆) δ=9.12 (d, J=2.4 Hz, 1H), 8.31 (dd, J=8.8, 2.4 Hz, 1H), 8.13 (d, J=8.4 Hz, 1H), 5.43 (s, 1H), 3.88 (s, 3H), 2.15 (s, 3H).

Step 3. To a solution of methyl 5-(5-hydroxy-3-methyl-1H-pyrazol-1-yl)picolinate S3 (200 mg, 0.858 mmol) in THF (2.00 mL) and water (2.00 mL), LiOH·H₂O (74.0 mg, 1.80 mmol) was added at 25° C., and the mixture was stirred for 2 hrs. The reaction mixture was concentrated to remove THF and the residue was purified by prep-HPLC (column: Xtimate C18 150*40 mm*10 μm; condition: water (NH₄HCO₃)-ACN; begin B: 0-6%) to give 5-(5-hydroxy-3-methyl-1H-pyrazol-1-yl)picolinic acid S4 (80.0 mg, 42.6% yield) as a yellow solid. ¹H NMR (400 MHz, DMSO-d₆) δ=9.34 (d, J=2.0 Hz, 1H), 8.41 (dd, J=8.8, 2.4 Hz, 1H), 7.80 (d, J=8.4 Hz, 1H), 4.31 (s, 1H), 1.90 (s, 3H).

Step 4. To a solution of 5-(5-hydroxy-3-methyl-1H-pyrazol-1-yl)picolinic acid S4 (45.0 mg, 0.204 mmol) in DCM (5.00 mL), DIEA (0.100 mL, 0.612 mmol), HATU (116 mg, 0.306 mmol) and 3-(3-(4-(aminomethyl)phenyl)-5-phenyl-3H-imidazo[4,5-b]pyridin-2-yl)pyridin-2-amine A4 (80.0 mg, 0.204 mmol) were added at 25° C., and the mixture stirred for 16 hrs. The reaction mixture was concentrated directly and the residue was purified by flash silica gel chromatography (eluent with 0-11% of MeOH in DCM) and then purified by prep-HPLC (column: Xtimate C18 150*40 mm*10 μm; condition: water (HCl)-ACN; begin B: 10-50) to give N-(4-(2-(2-aminopyridin-3-yl)-5-phenyl-3H-imidazo[4,5-b]pyridin-3-yl)benzyl)-5-(5-hydroxy-3-methyl-1H-pyrazol-1-yl)picolinamide 1-19 (6.70 mg, 5.41% yield) as a yellow solid. ¹H NMR (400 MHz, DMSO-d₆) δ=9.45 (t, J=6.4 Hz, 1H), 9.15-9.05 (m, 1H), 8.58-8.25 (m, 4H), 8.16-8.09 (m, 2H), 8.08-8.01 (m, 3H), 7.89 (d, J=7.6 Hz, 1H), 7.56-7.45 (m, 6H), 7.43-7.38 (m, 1H), 6.89 (t, J=6.8 Hz, 1H), 5.45 (s, 1H), 4.61 (d, J=6.4 Hz, 2H), 2.15 (s, 3H). HPLC Rₜ=4.580 min in 8 min chromatography, purity 97.8%. LCMS Rₜ=1.167 min in 2 min chromatography, purity 98.6%, MS ESI calcd, for 593.23 [M+H]⁺ 594.23, found 594.2.

### Example 20. Synthesis of N-(4-(2-(2-aminopyridin-3-yl)-5-phenyl-3H-imidazo[4,5-b]pyridin-3-yl)benzyl)-4-(2H-tetrazol-5-yl)benzamide (I-20)

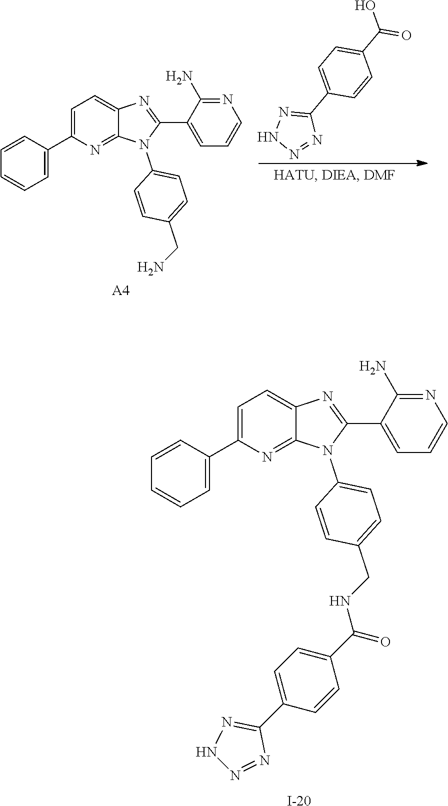

Step 1. To a solution of 3-(3-(4-(aminomethyl)phenyl)-5-phenyl-3H-imidazo[4,5-b]pyridin-2-yl)pyridin-2-amine A4 (200 mg, 0.510 mmol) in DMF (3.00 mL), DIEA (198 mg, 1.53 mmol), 4-(2H-tetrazol-5-yl) benzoic acid (97.0 mg, 0.510 mmol), and then HATU (252 mg, 0.660 mmol) were added and the mixture stirred at 25° C., for 2 hrs. The reaction was diluted with water (20 mL), extracted with EtOAc (20 mL×3) and the combined organic phase was washed with brine (30 mL×3), dried over anhydrous Na₂SO₄, filtered and concentrated. The residue was purified by prep-HPLC (Column: Welch Xtimate C18 150*30 mm*5 μm; mobile phase: [water (FA)-ACN]; B %: 8%-48%; 36 min) to give N-(4-(2-(2-aminopyridin-3-yl)-5-phenyl-3H-imidazo[4,5-b]pyridin-3-yl)benzyl)-4-(2H-tetrazol-5-yl)benzamide I-20 (32.2 mg, 10.9% yield) as a yellow solid. ¹H NMR (400 MHz, DMSO-d₆) δH=9.34 (t, J=6.0 Hz, 1H), 8.27 (d, J=8.4 Hz, 1H), 8.20-8.11 (m, 4H), 8.05-7.96 (m, 4H), 7.55-7.43 (m, 6H), 7.42-7.35 (m, 1H), 7.28-7.20 (m, 1H), 7.00 (s, 2H), 6.44 (dd, J=4.8, 7.6 Hz, 1H), 4.64 (d, J=6.0 Hz, 2H). HPLC Rₜ=2.352 min in 8 min chromatography, purity 97.7%. LCMS Rₜ=2.162 min in 4 min; Agilent PoroShell 120 EC-C18 2.7 μm 3.0*50 mm; purity 98.0%, MS ESI calcd, for 564.21, [M+H]⁺ 565.21, found 565.3.

### Example 21. Synthesis of N-(4-(2-(2-aminopyridin-3-yl)-5-phenyl-3H-imidazo[4,5-b]pyridin-3-yl)benzyl)-3-(2H-tetrazol-5-yl)benzamide (I-21)

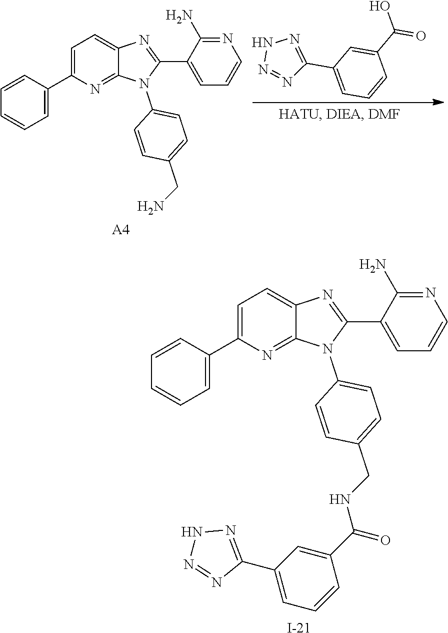

Step 1. To a solution of 3-(3-(4-(aminomethyl)phenyl)-5-phenyl-3H-imidazo[4,5-b]pyridin-2-yl)pyridin-2-amine A4 (200 mg, 0.510 mmol) in DMF (3.00 mL), DIEA (198 mg, 1.53 mmol), 3-(2H-tetrazol-5-yl) benzoic acid (97.0 mg, 0.510 mmol), and then HATU (252 mg, 0.660 mmol) were added and the mixture stirred at 25° C., for 2 hrs. The reaction was diluted with water (20 mL) and extracted with EtOAc (20 mL×3). The combined organic phase was washed with brine (30 mL×3), dried over anhydrous Na₂SO₄, filtered and concentrated. The crude product was purified by combine-flash column (EtOAc in PE=55%) and then purified by prep-HPLC (Column: Welch Xtimate C18 150*30 mm*5 μm; mobile phase: [water (FA)-ACN]; B %: 8%-48%; 36 min) to give N-(4-(2-(2-aminopyridin-3-yl)-5-phenyl-3H-imidazo[4,5-b]pyridin-3-yl)benzyl)-3-(2H-tetrazol-5-yl)benzamide I-21 (52.6 mg, 18.0% yield) as a yellow solid. ¹H NMR (400 MHz, DMSO-d₆)⁶H=9.40 (t, J=6.0 Hz, 1H), 8.63 (s, 1H), 8.27 (d, J=8.4 Hz, 1H), 8.24-8.08 (m, 3H), 8.06-7.96 (m, 4H), 7.80-7.70 (m, 1H), 7.55-7.43 (m, 6H), 7.41-7.33 (m, 1H), 7.25 (dd, J=2.0, 7.6 Hz, 1H), 7.00 (s, 2H), 6.44 (dd, J=4.8, 7.6 Hz, 1H), 4.65 (d, J=6.0 Hz, 2H). HPLC Rₜ=2.395 min in 8 min chromatography, purity 98.6%. LCMS Rₜ=2.188 min in 4 min; Agilent PoroShell 120 EC-C18 2.7 μm 3.0*50 mm; purity 99.0%, MS ESI calcd, for 564.21, [M+H]⁺ 565.21, found 565.2.

### Example 22. Synthesis of N-[[6-[2-(2-amino-3-pyridyl)-5-phenyl-imidazo[4,5-b]pyridin-3-yl]-3-pyridyl]methyl]-4-(5-hydroxy-3-methyl-pyrazol-1-yl)benzamide (I-22)

### Synthesis of tert-butyl ((6-aminopyridin-3-yl)methyl)carbamate T2

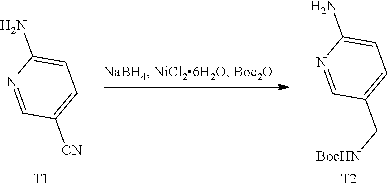

### Synthesis of N-[[6-[2-(2-amino-3-pyridyl)-5-phenyl-imidazo[4,5-b]pyridin-3-yl]-3-pyridyl]methyl]-4-(5-hydroxy-3-methyl-pyrazol-1-yl)benzamide (I-22)

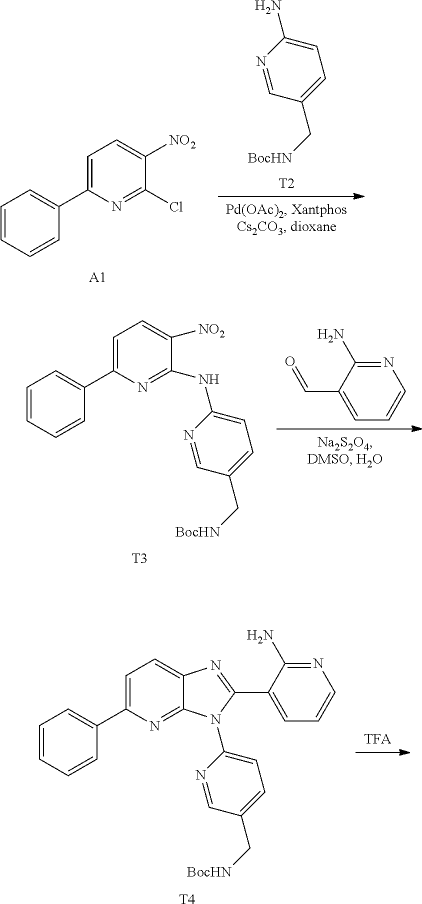

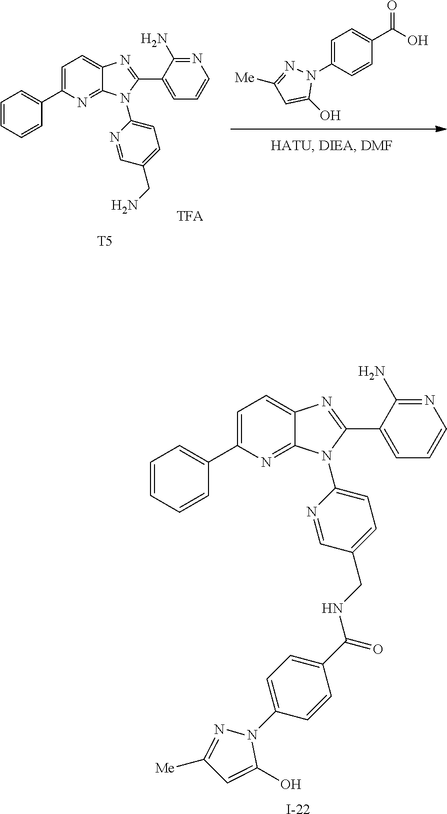

Step 1. To a solution of 6-aminopyridine-3-carbonitrile T1 (5.00 g, 42.0 mmol) in methanol (250 mL), NiCl₂·6H₂O (998 mg, 4.20 mmol), tert-butoxycarbonyl tert-butyl carbonate (18.3 g, 83.9 mmol), and then NaBH₄ (9.53 g, 252 mmol) were added to the reaction mixture slowly at 0° C., and the reaction mixture stirred at 0° C. to 25° C., for 16 hrs. The reaction mixture was poured slowly into ice water (200 mL) and concentrated to remove MeOH. The resulting mixture was extracted with EtOAc (150 mL×3) and the combined organic phase washed with brine (50 mL), water (50 mL), dried over anhydrous Na₂SO₄, filtered and concentrated. The residue was purified by flash silica gel chromatography (eluent with 0-4% of MeOH in DCM) to give tert-butyl N-[(6-amino-3-pyridyl)methyl]carbamate T2 (1.80 g, 19.2% yield) as yellow oil. LCMS Rₜ=0.345 min in 1.0 min chromatography, purity 67.3%, MS ESI calcd, for 223.14 [M+H]⁺ 224.14, found 224.1.

Step 2. To a solution of 2-chloro-3-nitro-6-phenyl-pyridine A1 (1.80 g, 7.67 mmol) and tert-butyl N-[(6-amino-3-pyridyl)methyl]carbamate T2 (1.80 g, 8.06 mmol) in 1,4-dioxane (30.0 mL), Xantphos (444 mg, 0.767 mmol), Cs₂CO₃ (7.50 g, 23.0 mmol) and Pd(OAc)₂ (172 mg, 0.767 mmol) were added at 25° C., and the mixture stirred at 90° C., for 16 hrs. The reaction mixture was concentrated, water (100 mL) was added to the residue and the resulting mixture extracted with EtOAc (50 mL×3). The combined organic phase was washed with brine (20 mL), water (20 mL), dried over anhydrous Na₂SO₄, filtered and concentrated. The residue was purified by flash silica gel chromatography (eluent with 0-37% of EtOAc in PE) to give tert-butyl N-[[6-[(3-nitro-6-phenyl-2-pyridyl)amino]-3-pyridyl]methyl]carbamate T3 (2.30 g, 71.1% yield) as a yellow solid. LCMS Rₜ=0.639 min in 1.0 min chromatography, purity 97.2%, MS ESI calcd, for 421.18 [M+H]⁺ 422.18, found 422.1.

Step 3. To a solution of tert-butyl N-[[6-[(3-nitro-6-phenyl-2-pyridyl)amino]-3-pyridyl]methyl]carbamate T3 (1.00 g, 2.37 mmol) in DMSO (23.0 mL) and methanol (23.0 mL), 2-aminopyridine-3-carbaldehyde (319 mg, 2.61 mmol) and Na₂S₂O₄ (1.03 g, 5.93 mmol) were added at 25° C., and the mixture stirred at 100° C., for 16 hrs. The reaction mixture was concentrated to remove MeOH, water (50 mL) was added to the residue and the resulting mixture extracted with EtOAc (100 mL×3). The combined organic phase was washed with brine (20 mL), water (20 mL), dried over anhydrous Na₂SO₄, filtered and concentrated. The residue was purified by flash silica gel chromatography (eluent with 0-49% of EtOAc in PE) to give tert-butyl ((6-(2-(2-aminopyridin-3-yl)-5-phenyl-3H-imidazo[4,5-b]pyridin-3-yl)pyridin-3-yl)methyl)carbamate T4 (420 mg, 45.0% yield) as a yellow solid. 1H NMR (400 MHz, DMSO-d₆) δ=8.55 (d, J=8.4 Hz, 2H), 8.43 (d, J=4.0 Hz, 1H), 8.23 (s, 1H), 8.17 (d, J=7.6 Hz, 2H), 7.94 (d, J=8.4 Hz, 1H), 7.67 (d, J=8.8 Hz, 1H), 7.53 (t, J=7.2 Hz, 2H), 7.48-7.38 (m, 2H), 7.12 (dd, J=4.8, 7.6 Hz, 1H), 4.11 (d, J=6.0 Hz, 2H), 1.40 (s, 9H).

Step 4. To a solution of tert-butyl ((6-(2-(2-aminopyridin-3-yl)-5-phenyl-3H-imidazo[4,5-b]pyridin-3-yl)pyridin-3-yl)methyl)carbamate T4 (100 mg, 0.203 mmol) in DCM (4.00 mL). TFA (2.00 mL) was added at 25° C., and the mixture stirred for 2 hrs. The reaction mixture was concentrated to give 3-[3-[5-(aminomethyl)-2-pyridyl]-5-phenyl-imidazo[4,5-b]pyridin-2-yl]pyridin-2-amine trifluoroacetate T5 (79.0 mg, 99.1% yield) as a brown oil, which was used in the next step directly. LCMS Rₜ=0.480 min in 1.0 min chromatography, purity 69.4%, MS ESI calcd, for 393.17 [M+H]⁺ 394.17, found 394.1.

Step 5. To a solution of 4-(5-hydroxy-3-methyl-1H-pyrazol-1-yl) benzoic acid (66.0 mg, 0.304 mmol) in DMF (4.00 mL), DIEA (149 mg, 1.15 mmol), HATU (219 mg, 0.577 mmol) and 3-[3-[5-(aminomethyl)-2-pyridyl]-5-phenyl-imidazo[4,5-b]pyridin-2-yl]pyridin-2-amine trifluoroacetate T5 (80.0 mg, 0.203 mmol) were added at 25° C., and the mixture stirred for 2 hrs. Aq. LiCl (20 mL, 3%) was added and the resulting mixture was extracted with EtOAc (10 mL×3). The combined organic phase was washed with brine (5 mL), water (5 mL), dried over anhydrous Na₂SO₄, filtered and concentrated. The residue was purified by flash silica gel chromatography (eluent with 0-13% of MeOH in DCM) and prep-HPLC (column: Xtimate C18 150*40 mm*10 μm; condition: water (NH₄HCO₃)-ACN; begin B: 4-44%) to give N-[[6-[2-(2-amino-3-pyridyl)-5-phenyl-imidazo[4,5-b]pyridin-3-yl]-3-pyridyl]methyl]-4-(5-hydroxy-3-methyl-pyrazol-1-yl)benzamide I-22 (4.50 mg, 3.62% yield) as a yellow solid. ¹H NMR (400 MHz, DMSO-d₆) δ=14.01-13.81 (m, 1H), 12.44-12.37 (m, 1H), 9.09-8.99 (m, 1H), 8.62-8.50 (m, 2H), 8.48-8.39 (m, 1H), 8.38-8.32 (m, 1H), 8.31-8.12 (m, 3H), 7.98-7.82 (m, 5H), 7.77 (s, 1H), 7.57-7.50 (m, 2H), 7.48-7.42 (m, 1H), 7.16-7.08 (m, 1H), 4.48 (d, J=5.6 Hz, 2H), 2.12 (s, 3H). HPLC Rₜ=2.824 min in 8 min chromatography, purity 96.7%. LCMS Rₜ=1.924 min in 4 min chromatography, purity 96.7%. MS ESI calcd, for 593.23 [M+H]⁺ 594.23, found 594.3.

### Example 23. Synthesis of N-(4-(2-(2-aminopyridin-3-yl)-6-phenyl-3H-imidazo[4,5-b]pyridin-3-yl)benzyl)-4-(5-hydroxy-3-methyl-1H-pyrazol-1-yl)benzamide (I-23)

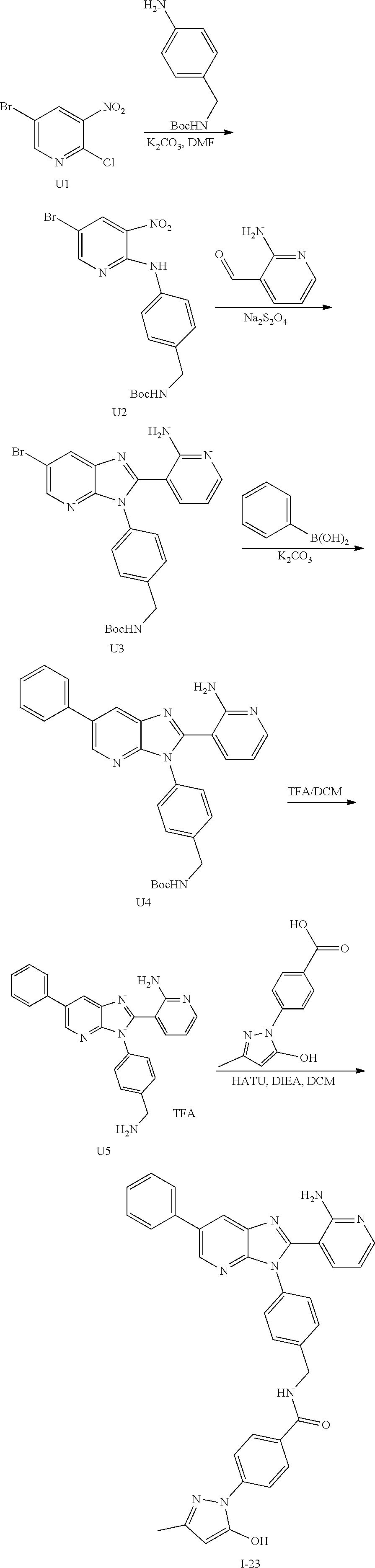

Step 1. To a solution of 5-bromo-2-chloro-3-nitro-pyridine U1 (5.00 g, 21.1 mmol) in DMSO (10.0 mL), tert-butyl N-[(4-aminophenyl)methyl]carbamate (4.92 g, 22.1 mmol) and DIEA (5.60 mL, 33.7 mmol) were added at 25° C., and the mixture stirred at 80° C., for 16 hrs. Water (20 mL) was added and the resulting mixture extracted with EtOAc (20 mL×3). The combined organic phase was washed with brine (10 mL), water (10 mL), dried over anhydrous Na₂SO₄, filtered and concentrated. The residue was purified by flash silica gel chromatography (eluent with 0-16% of EtOAc/PE) to give tert-butyl (4-((5-bromo-3-nitropyridin-2-yl)amino)benzyl)carbamate U2 (1.20 g, 67.3% yield) as a red solid. ¹H NMR (400 MHz, DMSO-d₆) δ=9.93 (s, 1H), 8.67 (d, J=2.4 Hz, 1H), 8.58 (d, J=2.4 Hz, 1H), 7.50 (d, J=8.4 Hz, 2H), 7.40 (t, J=6.0 Hz, 1H), 7.22 (d, J=8.4 Hz, 2H), 4.11 (d, J=6.0 Hz, 2H), 1.40 (s, 9H).

Step 2. To a solution of tert-butyl (4-((5-bromo-3-nitropyridin-2-yl)amino)benzyl)carbamate U2 (2.00 g, 4.73 mmol) in DMSO (12.0 mL) and methanol (12.0 mL), 2-aminopyridine-3-carbaldehyde (779 mg, 6.38 mmol) and Na₂S₂O₄ (2.06 mg, 11.8 mmol) were added at 25° C., and the mixture stirred at 100° C., for 16 hrs. The reaction mixture was concentrated to remove MeOH, water (10 mL) was subsequently added and the resulting mixture extracted with EtOAc (10 mL×3). The combined organic phase was washed with brine (10 mL), water (10 mL), dried over anhydrous Na₂SO₄, filtered and concentrated. The residue was purified by flash silica gel chromatography (eluent with 0~44% of EtOAc/PE) to give tert-butyl (4-(2-(2-aminopyridin-3-yl)-6-bromo-3H-imidazo[4,5-b]pyridin-3-yl)benzyl)carbamate U3 (400 mg, 17.1% yield) as yellow oil, ¹H NMR (400 MHz, DMSO-d₆) δ=8.50 (d, J=2.4 Hz, 1H), 8.40 (d, J=2.4 Hz, 1H), 8.02-7.98 (m, 1H), 7.78 (t, J=6.4 Hz, 1H), 7.55-7.36 (m, 4H), 7.26-7.21 (m, 1H), 6.95 (brs, 2H), 6.40 (dd, J=4.8 Hz, 7.6 Hz, 1H), 4.20 (d, J=6.0 Hz, 2H), 1.41 (s, 9H).

Step 3. To a solution of tert-butyl (4-(2-(2-aminopyridin-3-yl)-6-bromo-3H-imidazo[4,5-b]pyridin-3-yl)benzyl)carbamate U3 (400 mg, 0.807 mmol) and phenylboronic acid (197 mg, 1.61 mmol) in 1,4-dioxane (6.00 mL) and water (0.500 mL), K₂CO₃ (335 mg, 2.42 mmol) and Pd(dppf)Cl₂ (33.0 mg, 0.0404 mmol) were added at 25° C., and the mixture stirred at 90° C., for 16 hrs. The reaction mixture was concentrated directly and the residue was purified by flash silica gel chromatography (eluent with 0-5% of MeOH/DCM) to give tert-butyl (4-(2-(2-aminopyridin-3-yl)-6-phenyl-3H-imidazo[4,5-b]pyridin-3-yl)benzyl)carbamate U4 (316 mg, 79.5% yield) as brown oil. ¹H NMR (400 MHz, DMSO-d₆) δ=8.63-8.49 (m, 1H), 8.48-8.39 (m, 1H), 8.02-7.98 (m, 1H), 7.78 (d, J=7.6 Hz, 2H), 7.55-7.36 (m, 8H), 7.26-7.21 (m, 1H), 7.07-6.92 (m, 2H), 6.44-6.37 (m, 1H), 4.24-4.18 (m, 2H), 1.40 (s, 9H).

Step 4. To a solution of tert-butyl (4-(2-(2-aminopyridin-3-yl)-6-phenyl-3H-imidazo[4,5-b]pyridin-3-yl)benzyl)carbamate U4 (300 mg, 0.609 mmol) in DCM (2.00 mL), TFA (2.00 mL) was added at 25° C., and the mixture was stirred for 2 hrs. The reaction mixture was concentrated to give 3-(3-(4-(aminomethyl)phenyl)-6-phenyl-3H-imidazo[4,5-b]pyridin-2-yl)pyridin-2-amine trifluoroacetate U5 (300 mg, crude) as yellow oil, which was used in the next step directly. LCMS R₁=0.474 min in 1.0 min chromatography, purity 81.1%, MS ESI calcd, for 392.17 [M+H]⁺ 393.17, found 393.2.

Step 5. To a solution of 3-(3-(4-(aminomethyl)phenyl)-6-phenyl-3H-imidazo[4,5-b]pyridin-2-yl)pyridin-2-amine trifluoroacetate U5 (100 mg, 0.255 mmol) in DCM (5.00 mL), 4-(5-hydroxy-3-methyl-1H-pyrazol-1-yl)benzoic acid (83.0 mg, 0.382 mmol), DIEA (99.0 mg, 0.764 mmol) and HATU (145 mg, 0.382 mmol) were added at 25° C., and the mixture stirred for 3 hrs. The reaction mixture was concentrated directly and the residue was purified by flash silica gel chromatography (eluent with 0-100% of EtOAc in PE) to give N-[[4-[2-(2-amino-3-pyridyl)-6-phenyl-imidazo[4,5-b]pyridin-3-yl]phenyl]methyl]-4-(5-hydroxy-3-methyl-pyrazol-1-yl)benzamide I-23 (12.0 mg, 8.03% yield) as a yellow solid, ¹H NMR (400 MHz, DMSO-d₆) δ=9.19 (d, J=5.6 Hz, 1H), 8.72 (d, J=2.0 Hz, 1H), 8.54 (d, J=2.0 Hz, 1H), 8.17-8.08 (m, 1H), 8.05-7.91 (m, 3H), 7.89-7.75 (m, 4H), 7.58-7.47 (m, 6H), 7.47-7.39 (m, 1H), 6.91 (t, J=6.8 Hz, 1H), 5.43 (d, J=1.6 Hz, 1H), 4.58 (d, J=5.6 Hz, 2H), 2.14 (s, 3H). HPLC Rₜ=4.093 min in 8 min chromatography, purity 98.6%. LCMS Rₜ=1.932 min in 4 min chromatography, purity 99.4%, MS ESI calcd, for 592.23 [M+H]⁺ 593.23, found 593.5.

### Example 24. Synthesis of N-[[4-[2-(2-amino-3-pyridyl)-6-(3-pyridyl)imidazo[4,5-b]pyridin-3-yl]phenyl]methyl]-4-(5-hydroxy-3-methyl-pyrazol-1-yl)benzamide (I-24)

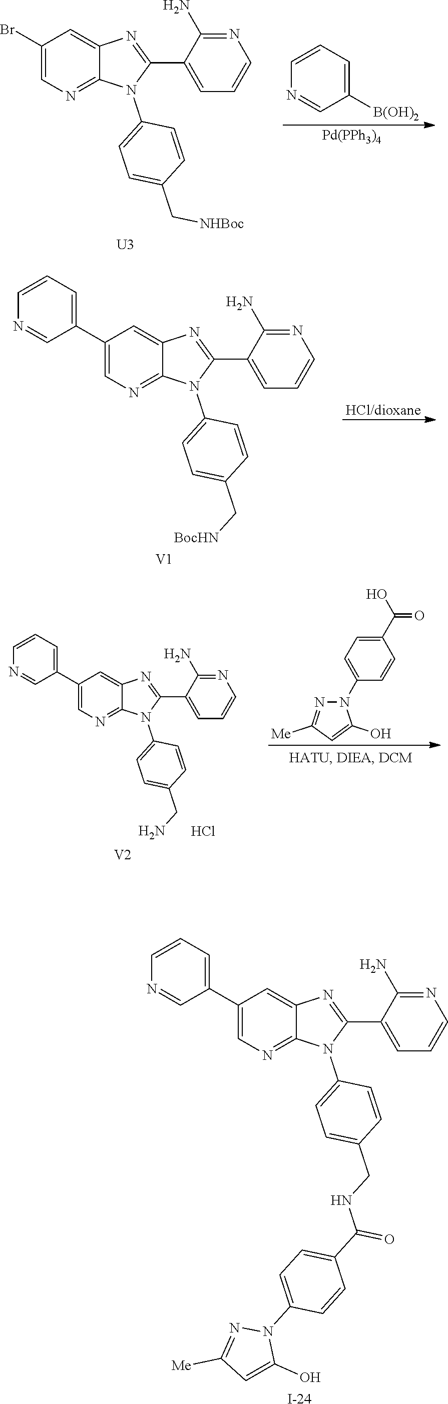

Step 1. Tert-butyl (4-(2-(2-aminopyridin-3-yl)-6-bromo-3H-imidazo[4,5-b]pyridin-3-yl)benzyl)carbamate U3 (330 mg, 0.666 mmol) and 3-pyridylboronic acid (246 mg, 2.00 mmol) were suspended in ethanol (5.00 mL) and toluene (5 mL). A solution of aqueous NaHCO₃ (1 mL, 1.1 M) was added and the mixture was degassed with nitrogen for 5 mins. Pd(PPh₃)₄ (38.0 mg, 0.0333 mmol) was added and the mixture was heated to 100° C., for 16 hrs. The reaction mixture was concentrated and the residue was purified by flash silica gel chromatography (eluent with 0-64% of EtOAc/PE) to give tert-butyl N-[[4-[2-(2-amino-3-pyridyl)-6-(3-pyridyl)imidazo[4,5-b]pyridin-3-yl]phenyl]methyl]carbamate V1 (101 mg, 30.7% yield) as brown oil. ¹H NMR (400 MHz, DMSO-d₆) δ=9.01 (d, J=2.0 Hz, 1H), 8.67 (d, J=2.0 Hz, 1H), 8.62 (dd, J=1.6, 4.8 Hz, 1H), 8.57 (d, J=2.0 Hz, 1H), 8.24-8.18 (m, 1H), 8.01 (dd, J=1.6, 4.8 Hz, 1H), 7.55 (dd, J=5.2, 7.6 Hz, 1H), 7.49 (t, J=4.8 Hz, 1H), 7.45-7.37 (m, 4H), 7.25 (dd, J=1.6, 7.6 Hz, 1H), 7.03 (s, 2H), 6.42 (dd, J=4.4, 7.2 Hz, 1H), 4.22 (d, J=6.0 Hz, 2H), 1.41 (s, 9H).

Step 2. To a solution of tert-butyl N-[[4-[2-(2-amino-3-pyridyl)-6-(3-pyridyl)imidazo[4,5-b]pyridin-3-yl]phenyl]methyl]carbamate V1 (100 mg, 0.203 mmol) in methanol (2.00 mL), HCl/dioxane (2.00 mL, 4 M) was added at 25° C., and the mixture stirred for 16 hrs. The reaction mixture was concentrated directly and the residue was purified by prep-HPLC (Column: Phenomenex C18 75*30 mm*3 μm; Condition: water (HCl)-ACN; Begin B: 0%-24%) to give 3-(3-(4-(aminomethyl)phenyl)-6-(pyridin-3-yl)-3H-imidazo[4,5-b]pyridin-2-yl)pyridin-2-amine hydrochloride V2 (35.0 mg, 43.7% yield) as yellow oil. ¹H NMR (400 MHz, DMSO-d₆) δ=9.27 (s, 1H), 8.87 (d, J=2.0 Hz, 1H), 8.83 (d, J=5.2 Hz, 1H), 8.79 (d, J=2.0 Hz, 1H), 8.70 (d, J=6.4 Hz, 1H), 8.62-8.24 (m, 5H), 8.16 (dd, J=1.6, 6.0 Hz, 1H), 7.95 (d, J=7.6 Hz, 2H), 7.72-7.66 (m, 2H), 7.64-7.58 (m, 2H), 6.90 (t, J=6.8 Hz, 1H), 4.12 (d, J=6.0 Hz, 2H). HPLC Rₜ=1.354 min in 8 min chromatography, purity 99.3%. LCMS Rₜ=0.959 min in 2 min chromatography, purity 98.6%, MS ESI calcd, for 393.17 [M+H]⁺ 394.17, found 394.0.

Step 3. To a solution of 3-(3-(4-(aminomethyl)phenyl)-6-(pyridin-3-yl)-3H-imidazo[4,5-b]pyridin-2-yl)pyridin-2-amine hydrochloride V2 (100 mg, 0.254 mmol) in DCM (3.00 mL), 4-(5-hydroxy-3-methyl-1H-pyrazol-1-yl)benzoic acid (99.0 mg, 0.762 mmol), DIEA (99.0 mg, 0.762 mmol) and HATU (145 mg, 0.381 mmol) were added at 25° C., and the mixture stirred for 3 hrs. The reaction mixture was concentrated directly and the residue purified by flash silica gel chromatography (eluent with 0-11% of MeOH in DCM) and then purified by flash silica gel chromatography (eluent with 0~100% of EtOAc in PE) to give N-[[4-[2-(2-amino-3-pyridyl)-6-(3-pyridyl)imidazo[4,5-b]pyridin-3-yl]phenyl]methyl]-4-(5-hydroxy-3-methyl-pyrazol-1-yl)benzamide I-24 (19.0 mg, 12.6% yield) as a light yellow solid. ¹H NMR (400 MHz, DMSO-d₆) δ=9.42-9.25 (m, 1H), 9.17 (t, J=5.6 Hz, 1H), 8.95-8.73 (m, 4H), 8.67-8.19 (m, 2H), 8.17-8.10 (m, 1H), 8.08-7.91 (m, 4H), 7.86 (d, J=8.8 Hz, 2H), 7.52 (s, 4H), 6.91 (t, J=6.8 Hz, 1H), 5.40 (s, 1H), 4.58 (d, J=6.0 Hz, 2H), 2.14 (s, 3H). HPLC Rₜ=2.460 min in 8 min chromatography, purity 98.7%. LCMS R₁=0.828 min in 2.5 min chromatography, purity 96.6%. MS ESI calcd, for 593.23 [M+H]⁺ 594.23, found 594.4.

### Example 25. Synthesis of (3-(4-(2-(2-aminopyridin-3-yl)-5-phenyl-3H-imidazo[4,5-b]pyridin-3-yl)phenyl)azetidin-1-yl)(4-(5-hydroxy-3-methyl-1H-pyrazol-1-yl)phenyl) methanone (I-25)

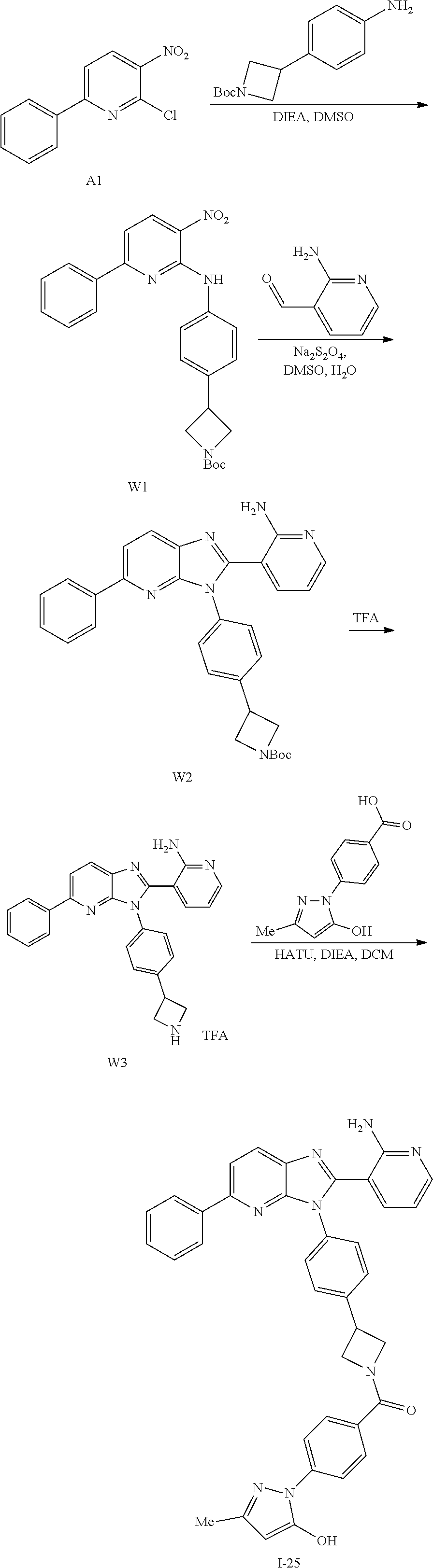

Step 1. To a solution of 2-chloro-3-nitro-6-phenyl-pyridine A1 (1.80 g, 7.67 mmol) in DMSO (15.0 mL), tert-butyl 3-(4-aminophenyl)azetidine-1-carboxylate (2.00 g, 8.05 mmol) and DIEA (2.10 mL, 12.3 mmol) were added at 25° C., and the mixture stirred at 80° C., for 16 hrs. Water (50 mL) was added to the residue and the resulting mixture was extracted with EtOAc (20 mL×3). The combined organic phase was washed with brine (50 mL), water (50 mL), dried over anhydrous Na₂SO₄, filtered and concentrated. The residue was purified by flash silica gel chromatography (eluent with 0-33% of EtOAc in PE) to give tert-butyl 3-(4-((3-nitro-6-phenylpyridin-2-yl)amino)phenyl)azetidine-1-carboxylate W1 (1.20 g, 67.3% yield) as a red solid. ¹H NMR (400 MHz, CHLOROFORM-d) 5=10.31 (s, 1H), 8.59 (d, J=8.8 Hz, 1H), 8.10-8.00 (m, 2H), 7.76 (d, J=8.4 Hz, 2H), 7.57-7.45 (m, 3H), 7.38 (d, J=8.4 Hz, 2H), 7.31 (d, J=8.8 Hz, 1H), 4.36 (t, J=8.8 Hz, 2H), 4.02 (dd, J=8.4, 6.0 Hz, 2H), 3.82-3.73 (m, 1H), 1.49 (s, 9H).

Step 2. To a solution of tert-butyl 3-(4-((3-nitro-6-phenylpyridin-2-yl)amino)phenyl) azetidine-1-carboxylate W1 (2.30 g, 5.15 mmol) in DMSO (23.0 mL) and methanol (23.0 mL) 2-aminonicotinaldehyde (849 mg, 6.95 mmol) and Na₂S₂O₄ (1.79 g, 10.3 mmol) were added at 25° C., and the mixture stirred at 100° C., for 16 hrs. The reaction mixture was concentrated to remove MeOH and water (50 mL) was subsequently added to the residue. The resulting mixture was extracted with EtOAc (100 mL×3). The combined organic phase was washed with brine (20 mL), water (20 mL), dried over anhydrous Na₂SO₄, filtered and concentrated. The residue was purified by flash silica gel chromatography (eluent with 0-66% of EtOAc in PE) to give tert-butyl 3-(4-(2-(2-aminopyridin-3-yl)-5-phenyl-3H-imidazo[4,5-b]pyridin-3-yl)phenyl)azetidine-1-carboxylate W2 (400 mg, 17.1% yield) as yellow oil, ¹H NMR (400 MHz, DMSO-d₆) δ=8.74 (d, J=2.4 Hz, 1H), 8.71 (d, J=2.4 Hz, 1H), 8.34-8.28 (m, 3H), 8.08 (dd, J=8.4, 2.0 Hz, 2H), 7.94 (dd, J=4.8, 2.0 Hz, 1H), 7.51 (d, J=8.4 Hz, 2H), 7.39 (dd, J=8.4, 6.4 Hz, 4H), 7.13 (dd, J=7.6, 1.6 Hz, 1H), 6.49-6.43 (m, 1H), 3.17 (s, 2H), 3.16 (s, 2H), 3.05 (s, 1H), 1.40 (s, 9H).

Step 3. To a solution of tert-butyl 3-(4-(2-(2-aminopyridin-3-yl)-5-phenyl-3H-imidazo[4,5-b]pyridin-3-yl)phenyl)azetidine-1-carboxylate W2 (200 mg, 0.390 mmol) in DCM (4.00 mL), TFA (2.00 mL) was added at 25° C., and the mixture stirred for 2 hrs. The reaction mixture was concentrated to give 3-(3-(4-(azetidin-3-yl)phenyl)-5-phenyl-3H-imidazo[4,5-b]pyridin-2-yl)pyridin-2-amine trifluoroacetate W3 (161 mg, 99.8% yield) as brown oil, which was used in the next step directly. LCMS Rₜ=0.410 min in 1.0 min chromatography, purity 79.5%, MS ESI calcd, for 418.19 [M+H]⁺ 419.19, found 419.1.

Step 4. To a solution of 3-(3-(4-(azetidin-3-yl)phenyl)-5-phenyl-3H-imidazo[4,5-b]pyridin-2-yl)pyridin-2-amine trifluoroacetate W3 (100 mg, 0.240 mmol) in DCM (5.00 mL), 4-(5-hydroxy-3-methyl-1H-pyrazol-1-yl)benzoic acid (52.0 mg, 0.240 mmol), DIEA (93.0 mg, 0.720 mmol), and then HATU (109 mg, 0.280 mmol) were added to the mixture, and the mixture was stirred at 25° C., for 12 hrs. The reaction mixture was concentrated and purified by prep-HPLC (Column: Welch Xtimate C18 150*30 mm*5 μm; mobile phase: [water (FA)-ACN]; B %: 12%-52%; 25 min) to give (3-(4-(2-(2-aminopyridin-3-yl)-5-phenyl-3H-imidazo[4,5-b]pyridin-3-yl)phenyl)azetidin-1-yl)(4-(5-hydroxy-3-methyl-1H-pyrazol-1-yl)phenyl)methanone I-25 (8.70 mg, 5.80% yield) as a yellow solid. ¹H NMR (400 MHz, DMSO-d₆) δ_{H}=11.74 (s, 1H), 8.27 (d, J=8.4 Hz, 1H), 8.06-7.97 (m, 4H), 7.88-7.73 (m, 4H), 7.64-7.57 (m, 2H), 7.54-7.43 (m, 4H), 7.42-7.36 (m, 1H), 7.25-7.19 (m, 1H), 6.95 (s, 2H), 6.43 (dd, J=4.8, 7.6 Hz, 1H), 5.41 (s, 1H), 4.83-4.70 (m, 1H), 4.60-4.41 (m, 2H), 4.21-4.01 (m, 2H), 2.18-2.08 (m, 3H). HPLC Rₜ=2.585 min in 8 min chromatography, purity 99.8%. LCMS Rₜ=2.670 min in 4 min; Agilent PoroShell 120 EC-C18 2.7 μm 3.0*50 mm; purity 100%, MS ESI calcd, for 618.25, [M+H]⁺ 618.25, found 619.3.

### Example 26. Synthesis of (4-(2H-tetrazol-5-yl)phenyl)(3-(4-(2-(2-aminopyridin-3-yl)-5-phenyl-3H-imidazo[4,5-b]pyridin-3-yl)phenyl)azetidin-1-yl)methanone (I-26)

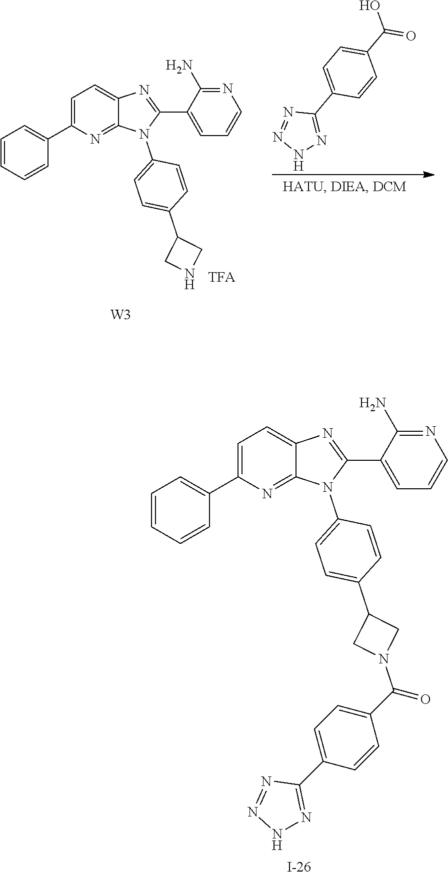

Step 1. To a solution of 3-(3-(4-(azetidin-3-yl)phenyl)-5-phenyl-3H-imidazo[4,5-b]pyridin-2-yl)pyridin-2-amine trifluoroacetate W3 (100 mg, 0.240 mmol) in DCM (10.0 mL), 4-(2H-tetrazol-5-yl)benzoic acid (68.0 mg, 0.356 mmol), DIEA (93.0 mg, 0.720 mmol) and HATU (136 mg, 0.360 mmol) were added at 25° C., and the mixture stirred for 2 hrs. The reaction mixture was concentrated and the residue was purified by prep-HPLC (Column: Xtimate C18 150*40 mm*10 μm; condition: water (HCl)-ACN; begin B: 18-58) to give (4-(2H-tetrazol-5-yl)phenyl)(3-(4-(2-(2-aminopyridin-3-yl)-5-phenyl-3H-imidazo[4,5-b]pyridin-3-yl)phenyl)azetidin-1-yl)methanone I-26 (41.0 mg, 27.5% yield) as a yellow solid. ¹H NMR (400 MHz, DMSO-d₆) δ=8.50-8.30 (m, 2H), 8.20-7.97 (m, 6H), 7.97-7.84 (m, 3H), 7.68-7.56 (m, 4H), 7.53-7.39 (m, 3H), 6.94-6.86 (m, 1H), 4.77 (t, J=8.4 Hz, 1H), 4.61-4.49 (m, 2H), 4.17-4.07 (m, 2H). HPLC Rₜ=3.710 min in 8 min chromatography, purity 95.8%. LCMS R₁=1.170 min in 2.5 min chromatography, purity 96.1%, MS ESI calcd, for 590.23 [M+H]⁺ 591.23, found 591.4.

### Example 27. Synthesis of (3-(2H-tetrazol-5-yl)phenyl)(3-(4-(2-(2-aminopyridin-3-yl)-5-phenyl-3H-imidazo[4,5-b]pyridin-3-yl)phenyl)azetidin-1-yl)methanone (I-27)

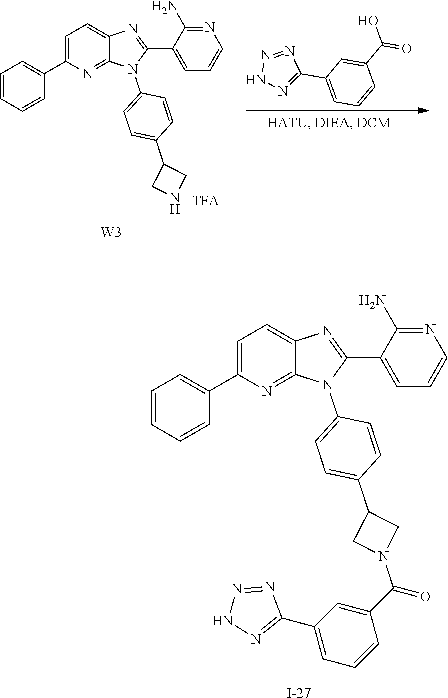

Step 1. To a solution of 3-(3-(4-(azetidin-3-yl)phenyl)-5-phenyl-3H-imidazo[4,5-b]pyridin-2-yl)pyridin-2-amine trifluoroacetate W3 (100 mg, 0.240 mmol) in DCM (10.0 mL), 3-(2H-tetrazol-5-yl)benzoic acid (68.0 mg, 0.360 mmol), DIEA (93.0 mg, 0.720 mmol) and HATU (136 mg, 0.360 mmol) were added at 25° C., and the mixture stirred for 2 hrs. The reaction mixture was concentrated and purified by prep-HPLC (Column: Xtimate C18 150*40 mm*10 μm; condition: water (HCl)-ACN; begin B: 18-58) to give (3-(2H-tetrazol-5-yl)phenyl)(3-(4-(2-(2-aminopyridin-3-yl)-5-phenyl-3H-imidazo[4,5-b]pyridin-3-yl)phenyl)azetidin-1-yl)methanone I-27 (11.0 mg, 7.73% yield) as a yellow solid. ¹H NMR (400 MHz, DMSO-d₆) δ=8.45-8.28 (m, 3H), 8.22 (d, J=7.6 Hz, 1H), 8.10 (d, J=6.0 Hz, 1H), 8.08-8.00 (m, 3H), 7.92 (d, J=7.8 Hz, 1H), 7.87 (d, J=4.0 Hz, 1H), 7.72 (t, J=7.6 Hz, 1H), 7.68-7.62 (m, 2H), 7.62-7.56 (m, 2H), 7.52-7.46 (m, 2H), 7.45-7.39 (m, 1H), 6.93-6.84 (m, 1H), 4.80 (t, J=8.8 Hz, 1H), 4.62-4.51 (m, 2H), 4.20-4.08 (m, 2H). HPLC Rₜ=3.773 min in 8 min chromatography, purity 98.3%. LCMS Rₜ=1.187 min in 2.5 min chromatography, purity 95.9%, MS ESI calcd, for 590.23 [M+H]⁺ 591.23, found 591.4.

### Example 28. Synthesis of N-[[4-[2-(2-amino-3-pyridyl)-6-phenyl-benzimidazol-1-yl]phenyl]methyl]-4-(5-hydroxy-3-methyl-pyrazol-1-yl)benzamide (I-28)

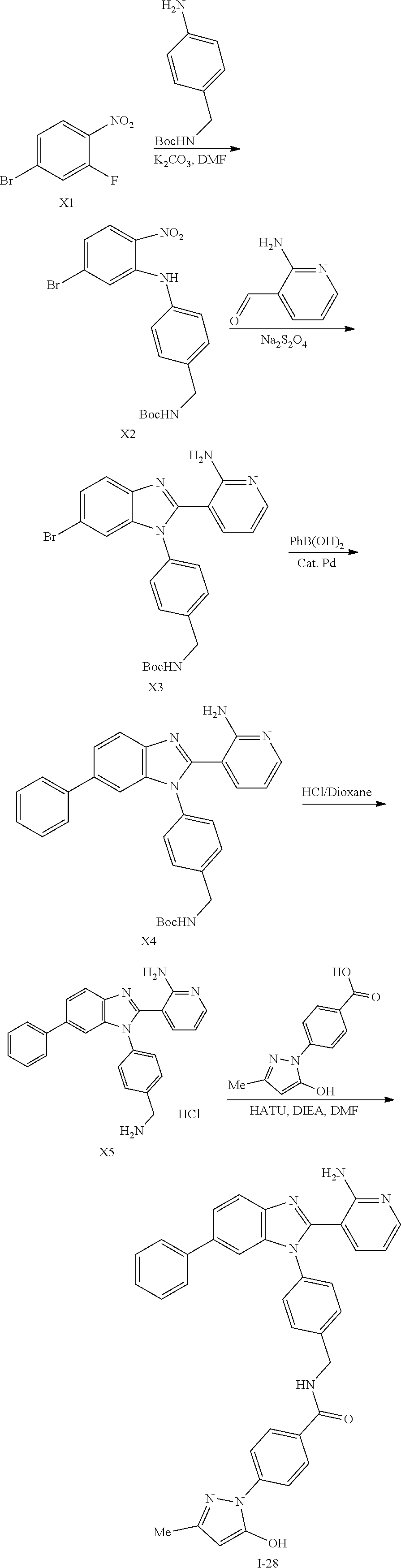

Step 1. To a solution of tert-butyl N-[(4-aminophenyl)methyl]carbamate (4.70 g, 21.5 mmol) and 4-bromo-2-fluoro-1-nitro-benzene X1 (4.50 g, 20.5 mmol) in DMF (20.0 mL), K₂CO₃ (5.65 mg, 40.9 mmol) was added and the mixture stirred at 90° C., for 5 hrs. The resulting mixture was allowed to cool to 25° C., diluted with EtOAc (100 mL) and washed with 5% aq. LiCl solution (200 mL×3). The organic layer was dried with Na₂SO₄, filtered and concentrated. The residue was purified by column chromatography (15% of EtOAc in PE) to give tert-butyl N-[[4-(5-bromo-2-nitro-anilino)phenyl]methyl]carbamate X2 (2.58 g, 29.9% yield) as a yellow solid. ¹H NMR (400 MHz, DMSO-d₆) δ=9.48 (s, 1H), 8.05 (d, J=9.2 Hz, 1H), 7.42 (s, 1H), 7.35-7.25 (m, 4H), 7.11 (d, J=1.6 Hz, 1H), 7.00 (dd, J=2.0, 8.8 Hz, 1H), 4.15 (d, J=6.0 Hz, 2H), 1.40 (s, 9H).

Step 2. To a stirred solution of 2-aminopyridine-3-carbaldehyde (195 mg, 1.60 mmol), tert-butyl N-[[4-(5-bromo-2-nitro-anilino)phenyl]methyl]carbamate X2 (500 mg, 1.18 mmol) in DMSO (10.0 mL) and methanol (2.0 mL), Na₂S₂O₄ (618 mg, 3.60 mmol) was added. The resulting mixture was stirred at 100° C. under N₂ for 12 hrs. The reaction mixture was diluted with EtOAc (50 mL) and washed with brine (50 mL×2), dried over anhydrous sodium sulfate, filtered and the filtrate was concentrated to afford a residue. The crude product was purified by flash chromatography on silica gel (60% of EtOAc in PE) to give tert-butyl N-[[4-[2-(2-amino-3-pyridyl)-6-bromo-benzimidazol-1-yl]phenyl]methyl]carbamate X3 (400 mg, 68.3% yield) as a yellow solid. LCMS Rₜ=0.50 min in 1.0 min chromatography, purity 98.1%, MS ESI calcd, for 493.11 [M+H]⁺ 494.11, found 494.1.

Step 3. To a stirred solution of phenylboronic acid (296 mg, 2.43 mmol), tert-butyl N-[[4-[2-(2-amino-3-pyridyl)-6-bromo-benzimidazol-1-yl]phenyl]methyl]carbamate X3 (400 mg, 0.810 mmol) and Cs₂CO₃ (791 mg, 2.43 mmol) in 1,4-dioxane (5.00 mL) and water (1.00 mL), Pd(dppf)C₁₂ (59.0 mg, 0.0810 mmol) was added under N₂ and the reaction stirred at 90° C. under N₂ for 12 hrs. The reaction mixture was concentrated to remove dioxane and diluted with EtOAc (10 mL), washed with brine (20 mL), dried over anhydrous sodium sulfate, filtered and the filtrate was concentrated. The residue was purified by flash chromatography on silica gel (70% of EtOAc in PE) to give tert-butyl N-[[4-[2-(2-amino-3-pyridyl)-6-phenyl-benzimidazol-1-yl]phenyl]methyl]carbamate X4 (150 mg, 37.7% yield) as a yellow solid. LCMS Rₜ=0.550 min in 1.0 min chromatography, purity 97.1%. MS ESI calcd, for 491.23 [M+H]⁺ 492.23, found 492.2.

Step 4. To a stirred solution of tert-butyl N-[[4-[2-(2-amino-3-pyridyl)-6-phenyl-benzimidazol-1-yl]phenyl]methyl]carbamate X4 (150 mg, 0.310 mmol) in 1,4-dioxane (2.00 mL) HCl/dioxane (2.00 mL, 4 M) was added and the reaction mixture was stirred at 25° C. under N₂ for 2 hrs. The reaction mixture was concentrated to afford 3-[1-[4-(aminomethyl)phenyl]-6-phenyl-benzimidazol-2-yl]pyridin-2-amine hydrochloride X5 (160 mg, crude) as a yellow solid. ¹H NMR (400 MHz, DMSO-d₆) δ=8.42 (d, J=2.4 Hz, 4H), 8.11 (dd, J=1.6, 6.0 Hz, 1H), 7.96 (d, J=8.4 Hz, 1H), 7.77-7.68 (m, 4H), 7.67-7.60 (m, 4H), 7.50-7.45 (m, 2H), 7.41-7.34 (m, 2H), 6.90-6.75 (m, 1H), 4.25-4.10 (m, 2H).

Step 5. To a stirred solution of 4-(5-hydroxy-3-methyl-pyrazol-1-yl)benzoic acid (11.0 mg, 0.0511 mmol) in DMF (2.00 mL), DIEA (20.0 mg, 0.150 mmol) and HATU (21.0 mg, 0.0500 mmol) were added and the reaction mixture stirred at 25° C., for 10 mins, 3-[1-[4-(Aminomethyl)phenyl]-6-phenyl-benzimidazol-2-yl]pyridin-2-amine hydrochloride X5 (20.0 mg, 0.0510 mmol) was then added to the reaction and the resulting mixture was stirred at 25° C., for 2 hrs. The reaction mixture was diluted with MeOH (1 mL) and purified by prep-HPLC (column: welch xtimate c18 150*25 mm*5 μm, method: water (NH₄HCO₃)-CAN, begin B: 30, end B: 60) to afford N-[[4-[2-(2-amino-3-pyridyl)-6-phenyl-benzimidazol-1-yl]phenyl]methyl]-4-(5-hydroxy-3-methyl-pyrazol-1-yl)benzamide I-28 (1.00 mg, 3.31% yield) as an off-white solid. ¹H NMR (400 MHz, DMSO-d₆) δ=11.75 (s, 1H), 9.14 (s, 1H), 8.04-7.94 (m, 3H), 7.93-7.79 (m, 3H), 7.66-7.58 (m, 3H), 7.56-7.39 (m, 6H), 7.38-7.28 (m, 2H), 7.15 (dd, J=1.6, 7.6 Hz, 1H), 7.04 (s, 2H), 6.39 (dd, J=4.8, 7.6 Hz, 1H), 5.41 (s, 1H), 4.61 (d, J=5.6 Hz, 2H), 2.13 (s, 3H). HPLC Rₜ=3.883 min in 8 min chromatography, purity 96.5%. LCMS Rₜ=2.367 min in 4 min chromatography, purity 97.6%, MS ESI calcd. for 591.24 [M+H]⁺ 592.24, found 592.2.

### Example 29. Synthesis of N-[[4-[2-(2-amino-3-pyridyl)-5-phenyl-imidazo[4,5-b]pyridin-3-yl]phenyl]methyl]-4-(5-hydroxy-1-methyl-pyrazol-4-yl)benzamide (I-29)

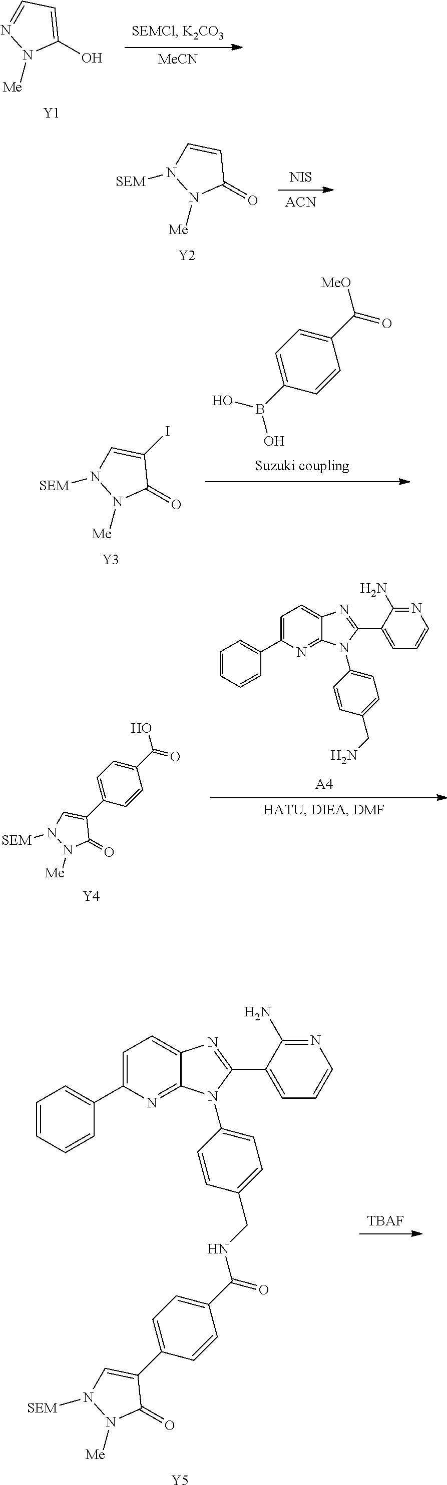

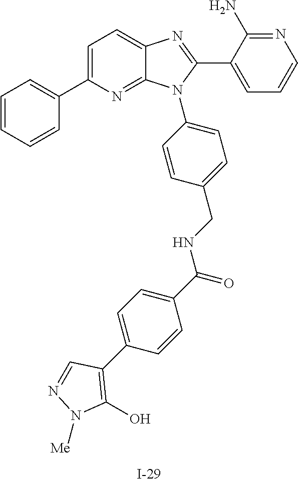

Step 1. To the solution of 2-methylpyrazol-3-ol Y1 (1.00 g, 10.2 mmol) in MeCN (30.0 mL), K₂CO₃ (3.10 g, 22.4 mmol) was added and stirred at 25° C., for 0.5 hr. A solution of 2-(chloromethoxy)ethyl-trimethyl-silane (3.10 mL, 17.3 mmol) in MeCN (30.0 mL) was then added to the reaction mixture at 25° C. under N₂. After stirring for 16 hrs, the reaction mixture was filtered and filtrate was concentrated in vacuum. The crude was purified by flash column (0-30% of MeOH in EtOAc) to give 2-methyl-1-(2-trimethylsilylethoxymethyl)pyrazol-3-one Y2 (1.30 g, 55.8% yield) as a yellow solid. ¹H NMR (400 MHz, DMSO-d₆) δ₁=7.85 (d, J=3.6 Hz, 1H), 5.26 (d, J=3.6 Hz, 1H), 5.11 (s, 2H), 3.41 (t, J=8.0 Hz, 2H), 3.24 (s, 3H), 0.81 (t, J=8.0 Hz, 2H), 0.04 (s, 9H).

Step 2. To a solution of 2-methyl-1-(2-trimethylsilylethoxymethyl)pyrazol-3-one Y2 (500 mg, 2.19 mmol) in MeCN (9.00 mL), NIS (473 mg, 2.10 mmol) was added at 0° C. The reaction was then warmed to 25° C., and stirred for 2 hrs. Water (20 mL) was then added to the reaction mixture and the whole extracted with EtOAc (30 mL×3). The organic layer was dried over Na₂SO₄, filtered and concentrated in vacuum. The crude was purified by flash column (0-8% of MeOH in EtOAc) to give 4-iodo-2-methyl-1-(2-trimethylsilylethoxymethyl)pyrazol-3-one Y3 (650 mg, 83.8% yield) as a white solid. ¹H NMR (400 MHz, DMSO-d₆) δ_{H}=8.13 (s, 1H), 5.12 (s, 2H), 3.43 (t, J=8.0 Hz, 2H), 3.31 (s, 3H), 0.80 (t, J=8.0 Hz, 2H), 0.04 (s, 9H).

Step 3. To a solution of 4-iodo-2-methyl-1-(2-trimethylsilylethoxymethyl)pyrazol-3-one Y3 (100 mg, 0.282 mmol) in ethanol (4.00 mL) and water (1.00 mL), (4-methoxycarbonylphenyl)boronic acid (61.0 mg, 0.339 mmol), XPhos (5.40 mg, 0.011 mmol), Pd₂(dba)₃ (5.20 mg, 0.006 mmol) and K₂CO₃ (98.0 mg, 0.706 mmol) were added under N₂. The reaction was stirred at 100° C., for 9 hrs. The reaction mixture was filtered and purified by prep-HPLC (column: Welch Xtimate C18 150*30 mm*5 μm; mobile phase: water (FA)-ACN; B %: 22%-52%, 25 min) to give 4-[2-methyl-3-oxo-1-(2-trimethylsilylethoxymethyl)pyrazol-4-yl]benzoic acid Y4 (66.0 mg, 67.1% yield). ¹H NMR (400 MHz, DMSO-d₆) δ_{H}=8.58 (s, 1H), 8.01 (d, J=8.4 Hz, 2H), 7.90 (d, J=8.4 Hz, 2H), 5.26 (s, 2H), 3.49 (t, J=8.0 Hz, 2H), 3.37 (s, 3H), 0.84 (1, J=8.0 Hz, 2H), 0.05 (s, 9H).

Step 4. To the solution of 3-(3-(4-(aminomethyl)phenyl)-5-phenyl-3H-imidazo[4,5-b]pyridin-2-yl)pyridin-2-amine A4 (74.0 mg, 0.189 mmol) in DMF (2.00 mL), HATU (86.0 mg, 0.227 mmol) and DIEA (0.100 mL, 0.568 mmol) were added and the reaction mixture stirred at 25° C., for 30 mins, 4-[2-methyl-3-oxo-1-(2-trimethylsilylethoxymethyl)pyrazol-4-yl]benzoic acid Y4 (66.0 mg, 0.189 mmol) was added and the mixture was stirred at 25° C. for 12 hrs. Water (10 ml) was added to the reaction mixture and the whole extracted with EtOAc (30 mL×3). The organic layer was washed with brine (10 mL×3), then dried over Na₂SO₄, filtered and concentrated in vacuum to give N-[[4-[2-(2-amino-3-pyridyl)-5-phenyl-imidazo[4,5-b]pyridin-3-yl]phenyl]methyl]-4-[2-methyl-3-oxo-1-(2-trimethylsilylethoxymethyl)pyrazol-4-yl]benzamide Y5 (120 mg, 87.6% yield) as a yellow solid. ¹H NMR (400 MHz, DMSO-d₆) δ_{H}=9.08 (t, J=6.4 Hz, 1H), 8.56 (s, 1H), 8.30-8.23 (m, 1H), 8.06-7.88 (m, 7H), 7.62-7.32 (m, 8H), 7.22 (dd, J=7.6, 2.0 Hz, 1H), 6.96 (s, 2H), 6.46-6.37 (m, 1H), 5.24 (s, 2H), 4.61 (d, J=6.0 Hz, 2H), 3.49 (t, J=8.0 Hz, 2H), 3.45 (s, 3H), 0.84 (t, J=8.0 Hz, 2H), 0.05 (s, 9H).

Step 5. A mixture of N-[[4-[2-(2-amino-3-pyridyl)-5-phenyl-imidazo[4,5-b]pyridin-3-yl]phenyl]methyl]-4-[2-methyl-3-oxo-1-(2-trimethylsilylethoxymethyl)pyrazol-4-yl]benzamide Y5 (120 mg, 0.166 mmol) and TBAF solution (2.00 mL, 1 M in THF) was stirred at 50° C., for 16 hrs. Water (10 mL) was added to the reaction mixture and the whole extracted with EtOAc (30 mL×3). The combined organic layer was dried over Na₂SO₄, filtered and concentrated in vacuum. The crude was purified by prep-HPLC (column: Welch Xtimate C18 150*25 mm*5 μm; mobile phase: water (NH₃H₂O)-ACN; B %: 25%-55%, 25 min) to give N-[[4-[2-(2-amino-3-pyridyl)-5-phenyl-imidazo[4,5-b]pyridin-3-yl]phenyl]methyl]-4-(5-hydroxy-1-methyl-pyrazol-4-yl)benzamide I-29 (14.0 mg, 13.5% yield) as a white solid. ¹H NMR (400 MHz, DMSO-d₆) δ_{H}=9.02 (t, J=6.0 Hz, 1H), 8.26 (d, J=8.4 Hz, 1H), 8.07-7.96 (m, 4H), 7.93-7.72 (m, 5H), 7.53-7.43 (m, 6H), 7.41-7.35 (m, 1H), 7.25-7.18 (m, 1H), 6.95 (s, 2H), 6.42 (dd, J=8.0, 5.2 Hz, 1H) 4.60 (d, J=6.0 Hz, 2H), 3.50 (s, 3H). LCMS Rₜ=2.483 min in 4 min chromatography, purity 100%, MS ESI calcd. For 592.23 [M+H]⁺ 593.23, found 593.1. HPLC Rₜ=4.696 min in 8 min chromatography, purity 98.1%.

### Example 30. Synthesis of (1r,4r)-N-(5-(2-(2-aminopyridin-3-yl)-5-phenyl-3H-imidazo[4,5-b]pyridin-3-yl)-6-methylpyridin-2-yl)-4-(5-oxo-4,5-dihydro-1,2,4-thiadiazol-3-yl)cyclohexane-1-carboxamide (I-38)

### Synthesis of (1r,4r)-4-(5-oxo-4,5-dihydro-1,2,4-thiadiazol-3-yl)cyclohexane-1-carboxylic acid

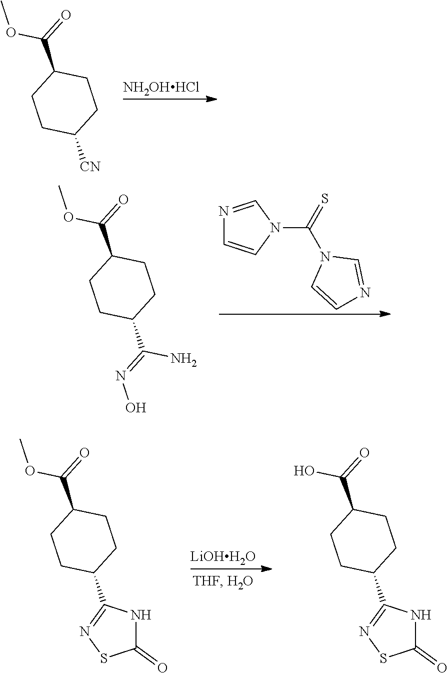

Step 1. To a solution of methyl (1r,4r)-4-cyanocyclohexane-1-carboxylate (2.00 g, 12.0 mmol) in ethanol (200 mL) was added NaHCO₃ (1.05 g, 12.0 mmol) and water (15.0 mL). Hydroxylamine hydrochloride (831 mg, 12.0 mmol) was then added and the reaction mixture was stirred at 85° C., for 16 hrs. The reaction mixture was concentrated directly and the mixture was diluted with H₂O (30 mL), extracted with EtOAc (40 mL×3), and washed with brine (30 mL). The organic layer was dried over Na₂SO₄ and concentrated to give methyl (1r,4r)-4-((Z)—N′-hydroxycarbamimidoyl)cyclohexane-1-carboxylate (2.00 g, 97.1% yield) as a light yellow solid, which was used in the next step without further purification. LCMS Rₜ=0.559 min in 2.5 min chromatography, purity 85.4%. MS ESI calcd. For 200.12 [M+H]⁺ 201.12, found 201.2.

Step 2. To a solution of methyl (1r,4r)-4-((Z)—N′-hydroxycarbamimidoyl)cyclohexane-1-carboxylate (2.00 g, 9.99 mmol) in THF (20.0 mL) was added di(imidazol-1-yl)methanethione (2.67 g, 15.0 mmol), and the mixture was stirred at 25° C., for 1 hr under N₂ atmosphere. The mixture was poured into water (50 mL) and extracted with DCM (50 mL×3). The organic phase was washed with brine (20 mL), dried with Na₂SO₄, concentrated, and the residue was dissolved in THF (30 mL). To the mixture was added BF₃·Et₂O (2.00 mL, 30.0 mmol) dropwise, and the mixture was stirred at 25° C., for 3 hrs under N₂ atmosphere. The mixture was diluted with water (50 mL) and extracted with EtOAc (50 mL×3). The organic phase was washed with brine (10 mL), dried with Na₂SO₄ and concentrated. The crude product was purified by prep-HPLC (column: Welch Xtimate C18 250*50 mm*10 μm; mobile phase: [water (FA)-ACN]; B %: 5%-50%, 20 min) to give methyl (1r,4r)-4-(5-oxo-4,5-dihydro-1,2,4-thiadiazol-3-yl)cyclohexane-1-carboxylate (1.0 g, 50.0% yield) as a yellow solid.

Step 3. To a solution of methyl (1r,4r)-4-(5-oxo-4,5-dihydro-1,2,4-thiadiazol-3-yl)cyclohexane-1-carboxylate (300 mg, 1.24 mmol) in THF (5.00 mL) and water (2.00 mL) was added hydroxylithium hydrate (156 mg, 3.71 mmol), and the reaction mixture was stirred at 25° C., for 1 hr. The reaction mixture was concentrated to give the crude product. The mixture was then diluted with H₂O (10 mL), extracted with PE (10 mL×3), and the pH of the aqueous phase was adjusted to pH~4 with HCl (2 M). The resulting mixture was extracted with DCM (10 mL×3), the combined organic phase was dried over anhydrous Na₂SO₄, filtered and concentrated to give (1r,4r)-4-(5-oxo-4,5-dihydro-1,2,4-thiadiazol-3-yl)cyclohexane-1-carboxylic acid (240 mg, 84.9% yield) as a white solid, which was used in the next step without further purification. LCMS Rₜ=0.793 min in 2.0 min chromatography, purity 88.5%, MS ESI calcd. For 228.06 [M+H]⁺ 229.06, found 229.0.

### Synthesis of (1r,4r)-N-(5-(2-(2-aminopyridin-3-yl)-5-phenyl-3H-imidazo[4,5-b]pyridin-3-yl)-6-methylpyridin-2-yl)-4-(5-oxo-4,5-dihydro-1,2,4-thiadiazol-3-yl)cyclohexane-1-carboxamide (I-38)

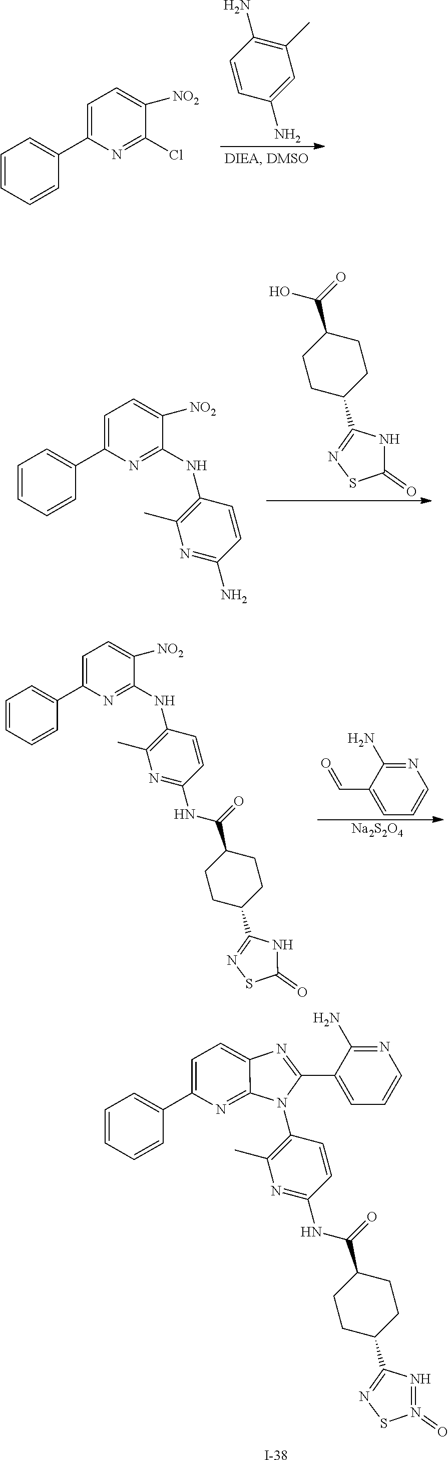

Step 1. To a solution of 2-chloro-3-nitro-6-phenylpyridine (4.00 g, 17.0 mmol) and 6-methylpyridine-2,5-diamine (2.52 g, 20.5 mmol) in DMSO (50.0 mL) was added DIEA (8.50 mL, 51.1 mmol) and the mixture was stirred at 80° C., for 12 hrs. Water (10 mL) was added to the residue and the resulting mixture was extracted with EtOAc (10 mL×3). The combined organic phase was washed with brine (20 mL), dried over anhydrous Na₂SO₄, filtered and concentrated. The residue was purified by flash silica gel chromatography (eluent of 0-10% MeOH in DCM) to give 6-methyl-N5-(3-nitro-6-phenylpyridin-2-yl)pyridine-2,5-diamine (5.38 g, 15.2 mmol) as a yellow solid. LCMS Rₜ=0.549 min in 1 min chromatography, purity 91.0%, MS ESI calcd, for 321.12, [M+H]⁺ 322.12, found 322.0.

Step 2. To a solution of 6-methyl-N5-(3-nitro-6-phenylpyridin-2-yl)pyridine-2,5-diamine (200 mg, 0.622 mmol) and (1r,4r)-4-(5-oxo-4,5-dihydro-1,2,4-thiadiazol-3-yl)cyclohexane-1-carboxylic acid (170 mg, 0.747 mmol) in pyridine (3.00 mL) was added EDCI (358 mg, 1.87 mmol) at 25° C. The reaction mixture was subsequently stirred at 85° C. for 2 hrs under N₂ atmosphere. The mixture was diluted with H₂O (30 mL), extracted with EtOAc (30 mL×3), and washed with brine (30 mL). The organic layer was dried over Na₂SO₄ and concentrated to give (1r,4r)-N-(6-methyl-5-((3-nitro-6-phenylpyridin-2-yl)amino)pyridin-2-yl)-4-(5-oxo-4,5-dihydro-1,2,4-thiadiazol-3-yl)cyclohexane-1-carboxamide (310 mg, 93.7% yield) as a red solid, which was used in the next step without further purification. LCMS Rₜ=1.519 min in 2.5 min chromatography, purity 67.7%. MS ESI calcd. For 531.17 [M+H]⁺ 532.17, found 532.2.

Step 3. To a solution of (1r,4r)-N-(6-methyl-5-((3-nitro-6-phenylpyridin-2-yl)amino)pyridin-2-yl)-4-(5-oxo-4,5-dihydro-1,2,4-thiadiazol-3-yl)cyclohexane-1-carboxamide (200 mg, 0.376 mmol) and 2-aminonicotinaldehyde (92.0 mg, 0.752 mmol) in DMSO (5.00 mL) and methanol (1.00 mL) was added Na₂S₂O₄ (393 mg, 2.26 mmol) at 25° C. The reaction mixture was stirred at 100° C., for 12 hrs. The mixture was diluted with saturated aqueous NaHCO₃ (20 mL), extracted with EtOAc (20 mL×3), washed with brine (20 mL) and the organic layer was dried over Na₂SO₄ and concentrated. The crude product was purified by prep-HPLC (column: Welch Xtimate C18 40*200 mm 7 μm; mobile phase: [water (FA)-ACN]; B %: 10%-50%, 25 min) to give (1r,4r)-N-(5-(2-(2-aminopyridin-3-yl)-5-phenyl-3H-imidazo[4,5-b]pyridin-3-yl)-6-methylpyridin-2-yl)-4-(5-oxo-4,5-dihydro-1,2,4-thiadiazol-3-yl)cyclohexane-1-carboxamide I-38 (82.4 mg, 35.5% yield) as a yellow solid. LCMS Rₜ=2.621 min in 4 min chromatography, purity 98.2%, MS ESI calcd. For 603.23 [M+H]⁺ 604.23, found 598.1. HPLC Rₜ=4.136 min in 8 min chromatography, purity 97.9%, 1H NMR (400 MHz, DMSO-d₆) δ=10.74-10.70 (m, 1H), 8.30 (d, J=8.4 Hz, 1H), 8.15-8.09 (m, 2H), 8.04-7.98 (m, 4H), 7.87 (d, J=8.8 Hz, 1H), 7.49-7.43 (m, 2H), 7.42-7.37 (m, 1H), 7.22-7.18 (m, 1H), 7.16 (s, 2H), 6.49-6.44 (m, 1H), 2.58-2.54 (m, 2H), 2.07 (s, 3H), 2.04-1.90 (m, 4H), 1.55-1.46 (m, 4H).

### Example 31. Synthesis of (1r,4r)-N-(5-(2-(2-aminopyridin-3-yl)-5-phenyl-3H-imidazo[4,5-b]pyridin-3-yl)-6-methylpyridin-2-yl)-4-(2H-tetrazol-5-yl)cyclohexane-1-carboxamide (I-39)

### Synthesis of (1r,4r)-4-(2-trityl-2H-tetrazol-5-yl)cyclohexane-1-carboxylic acid

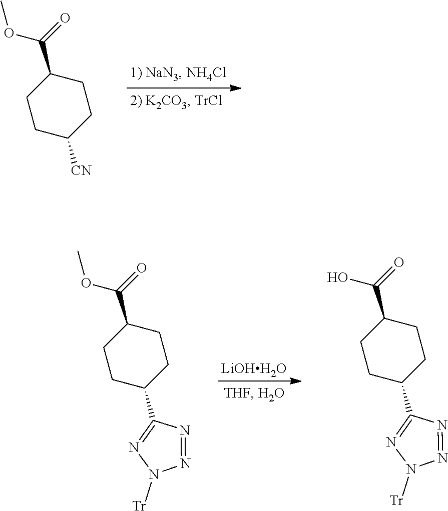

Step 1. To a solution of methyl (1r,4r)-4-cyanocyclohexane-1-carboxylate (500 mg, 2.99 mmol) in DMF (8.00 mL) was added NaN₃ (390 mg, 6.00 mmol) under N₂ atmosphere. The mixture was stirred at 110° C., for 12 hrs and the mixture was cooled to room temperature. TrCl (1.25 g, 4.49 mmol) and TEA (908 mg, 8.97 mmol) was added and the mixture stirred at 25° C., for 12 hrs. Water (5 mL) was added into the mixture and the resulting mixture was extracted with EtOAc (10 mL×3). The combined organic phase was washed with brine (15 mL), dried over anhydrous Na₂SO₄, filtered and concentrated. The residue was purified by combi-flash column (EtOAC in PE=0%-2%). The mixture was purified by prep-HPLC (column: Xtimate C18 200*40 mm*7 μm; mobile phase: [water (NH₄HCO₃)-ACN]; B %: 52%-92%, 35 min) to afford methyl (1r,4r)-4-(2-trityl-2H-tetrazol-5-yl)cyclohexane-1-carboxylate (162 mg, 43.0% yield) as a white solid. ¹H NMR (DMSO-d₆, 400 MHz) δ₁=7.45-7.30 (m, 9H), 7.09-6.87 (m, 6H), 3.60 (s, 3H), 2.98-2.87 (m, 1H), 2.43-2.35 (m, 1H), 2.08-2.02 (m, 2H), 2.01-1.94 (m, 2H), 1.61-1.42 (m, 4H).

Step 2. A solution of methyl (1r,4r)-4-(2-trityl-2H-tetrazol-5-yl)cyclohexane-1-carboxylate (60.0 mg, 0.354 mmol) and LiOH·H₂O (45.0 mg, 1.06 mmol) in THF (2.00 mL) and water (2.00 mL) was stirred at 25° C., for 2 hrs. Water (2 mL) was added and the mixture was then adjusted to pH~4 by aqueous HCl (2 M). The resulting mixture was extracted with DCM (5 mL×3). The combined organic phase was washed with brine (3 mL), water (3 mL), dried over anhydrous Na₂SO₄, filtered and concentrated to afford (1r,4r)-4-(2-trityl-2H-tetrazol-5-yl)cyclohexane-1-carboxylic acid (149 mg, 96% yield) as a white solid. ¹H NMR (DMSO-d₆, 400 MHz) δ_{H}=7.42-7.34 (m, 9H), 6.99 (dd, J=2.8, 6.4 Hz, 6H), 2.99-2.86 (m, 1H), 2.31-2.20 (m, 1H), 2.09-2.02 (m, 2H), 2.00-1.93 (m, 2H), 1.57-1.43 (m, 4H).

### Synthesis of (1r,4r)-N-(5-(2-(2-aminopyridin-3-yl)-5-phenyl-3H-imidazo[4,5-b]pyridin-3-yl)-6-methylpyridin-2-yl)-4-(2H-tetrazol-5-yl)cyclohexane-1-carboxamide (I-39)

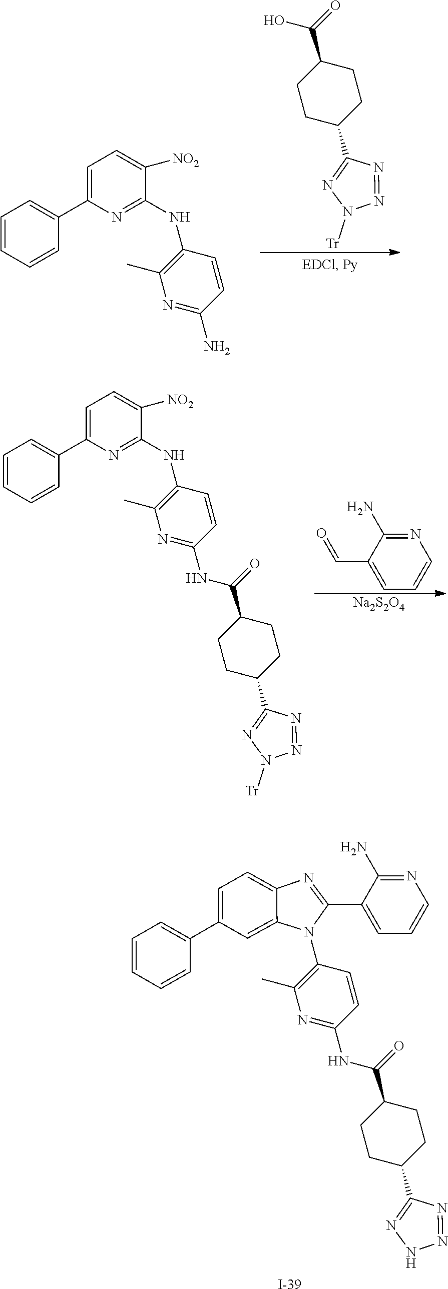

Step 1. To a solution of compound 6-methyl-N5-(3-nitro-6-phenylpyridin-2-yl)pyridine-2,5-diamine (55.0 mg, 0.170 mmol) in pyridine (1.00 mL) was added compound (1r,4r)-4-(2-trityl-2H-tetrazol-5-yl)cyclohexane-1-carboxylic acid (75.0 mg, 0.170 mmol) and EDCI (98.0 mg, 0.510 mmol) and the mixture was stirred at 80° C., for 2 hrs. The mixture was diluted with EtOAc (6 mL), and washed with brine (3 mL×3). The combined organic phase was dried over anhydrous Na₂SO₄, filtered and concentrated. The residue was purified by combi-flash column (EtOAC in PE=0%-20%) to afford (1r,4r)-N-(6-methyl-5-((3-nitro-6-phenylpyridin-2-yl)amino)pyridin-2-yl)-4-(2-trityl-2H-tetrazol-5-yl)cyclohexane-1-carboxamide (52.0 mg, 41% yield) as a red solid. ¹H NMR (DMSO-d₆, 400 MHz) δ_{H}=10.44 (s, 1H), 9.94 (s, 1H), 8.61 (d, J=8.4 Hz, 1H), 8.07-7.90 (m, 3H), 7.55 (d, J=8.4 Hz, 1H), 7.52-7.35 (m, 10H), 7.33-7.14 (m, 3H), 7.01 (dd, J=2.8, 5.6 Hz, 5H), 3.04-2.92 (m, 1H), 2.39 (s, 3H), 2.11 (d, J=10.8 Hz, 1H), 1.99-1.94 (m, 2H), 1.69-1.47 (m, 4H), 1.25-1.15 (m, 2H).

Step 2. To a solution of (1r,4r)-N-(6-methyl-5-((3-nitro-6-phenylpyridin-2-yl)amino)pyridin-2-yl)-4-(2-trityl-2H-tetrazol-5-yl)cyclohexane-1-carboxamide (52.0 mg, 0.0700 mmol) in DMSO (1.00 mL) and methanol (1.00 mL) was added 2-aminonicotinaldehyde (13.0 mg, 0.110 mmol) and Na₂S₂O₄ (36.0 mg, 0.210 mmol) and the mixture was stirred at 100° C., for 12 hrs. The mixture was diluted with EtOAc (5 mL), washed with brine (2 mL×3), and the combined organic phase was dried over anhydrous Na₂SO₄, filtered and concentrated. The mixture was purified by prep-HPLC (column: Xtimate C18 40*200 mm*7 μm; mobile phase: [water (HCl)-ACN]; B %: 6%-46%, 25 min) to afford (1r,4r)-N-(5-(2-(2-aminopyridin-3-yl)-5-phenyl-3H-imidazo[4,5-b]pyridin-3-yl)-6-methylpyridin-2-yl)-4-(2H-tetrazol-5-yl)cyclohexane-1-carboxamide I-39 (3.70 mg, 9% yield, HCl salt) as a yellow solid. ¹H NMR (DMSO-d₆, 400 MHz) δ_{H}=10.80 (s, 1H), 8.89-8.47 (m, 1H), 8.40 (d, J=8.4 Hz, 1H), 8.17-8.12 (m, 2H), 8.08 (d, J=8.4 Hz, 1H), 8.02 (d, J=7.2 Hz, 2H), 7.90 (d, J=8.8 Hz, 1H), 7.78 (dd, J=1.2, 7.6 Hz, 1H), 7.52-7.45 (m, 2H), 7.45-7.40 (m, 1H), 6.90 (dd, J=6.4, 7.6 Hz, 1H), 3.05-3.01 (m, 1H), 2.67-2.59 (m, 1H), 2.19 (s, 3H), 2.17-1.95 (m, 4H), 1.69-1.51 (m, 4H). HPLC Rₜ=2.512 min in 8 min chromatography, purity 99.4%. LCMS R₁=1.693 min in 4 min chromatography, purity 98.9%, MS ESI calcd, for 571.26 [M+H]⁺ 572.26, found 572.2.

### Example 32. Synthesis of N-(5-(2-(2-aminopyridin-3-yl)-5-phenyl-3H-imidazo[4,5-b]pyridin-3-yl)-6-methylpyridin-2-yl)-3-(5-oxo-4,5-dihydro-1,2,4-thiadiazol-3-yl)benzamide (I-40)

### Synthesis of 3-(5-oxo-4,5-dihydro-1,2,4-thiadiazol-3-yl)benzoic acid

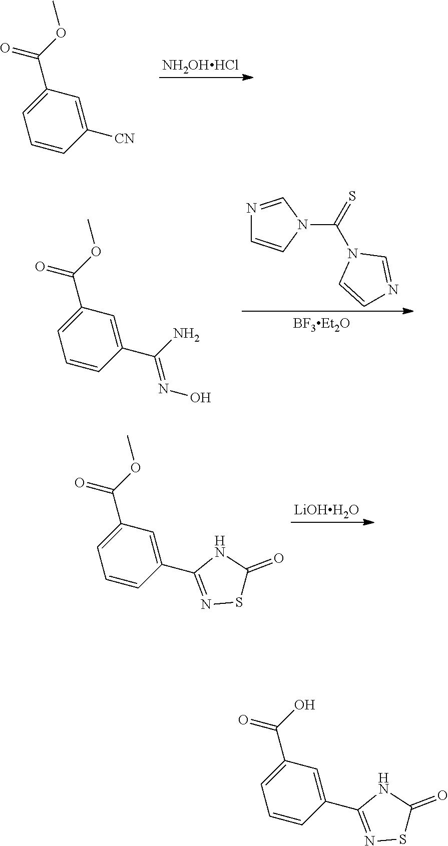

Step 1. To a solution of methyl 3-cyanobenzoate (2.00 g, 12.4 mmol) in methanol (20.0 mL) was added NaHCO₃ (1.15 g, 13.7 mmol) and hydroxylamine;hydrochloride (862 mg, 12.4 mmol) at 25° C. The mixture was stirred at 70° C., for 3 hrs. H₂O (30 mL) was then added to the reaction mixture and the whole extracted with EtOAc (60 mL×3). The organic layer was dried over Na₂SO₄ and concentrated to give methyl (Z)-3-(N′-hydroxycarbamimidoyl)benzoate (2.30 g, 95% yield) as a light yellow oil, which was used in the next step directly without further purification. ¹H NMR (DMSO-d₆, 400 MHz) δ_{H}=9.77 (s, 1H), 8.29 (s, 1H), 7.94 (t, J=8.0 Hz, 2H), 7.53 (t, J=7.6 Hz, 1H), 5.93 (s, 2H), 3.87 (s, 3H).

Step 2. To a solution of methyl (Z)-3-(N′-hydroxycarbamimidoyl)benzoate (1.00 g, 5.15 mmol) in THF (10.0 mL) was added di(1H-imidazol-1-yl)methanethione (1.38 g, 7.72 mmol) at 25° C., and the mixture was stirred for 1 hr. H₂O (10 mL) was then added to the mixture and the whole extracted with DCM (10 mL×3). The organic layer was washed with brine and dried over Na₂SO₄ to give a crude. The crude was dissolved in THF (10 mL), and then BF₃·Et₂O (2.19 g, 15.4 mmol) was added to the mixture. The resulting mixture was then stirred at 25° C. for 10 hrs. The reaction was quenched with saturated aqueous NaHCO₃ (30 mL) drop-wise. The resulting mixture was extracted with EtOAc (60 mL×3), the combined organic phase was washed with brine (50 mL), dried over anhydrous Na₂SO₄, filtered and concentrated. The crude product was purified by column chromatography on silica gel (EtOAc in PE=20%) to give methyl 3-(5-oxo-4,5-dihydro-1,2,4-thiadiazol-3-yl)benzoate (910 mg, 75% yield) as a white solid. ¹H NMR (DMSO-d₆ 400 MHz) δ_{H}=8.59-8.53 (m, 1H), 8.23-8.17 (m, 1H), 8.15-8.08 (m, 1H), 7.72-7.62 (m, 1H), 3.89 (s, 3H).

Step 3. To a solution of methyl 3-(5-oxo-4,5-dihydro-1,2,4-thiadiazol-3-yl)benzoate (767 mg, 3.25 mmol) in THF (4.00 mL) and water (4.00 mL) was added LiOH·H₂O (409 mg, 9.74 mmol), and the mixture was stirred at 25° C. for 16 hrs. Water (10 mL) was added to the reaction mixture, and the mixture was then adjusted to pH~3 by saturated aqueous HCl (2 M). The resulting white solid that precipitated was then filtered. The filter cake was concentrated to give 3-(5-oxo-4,5-dihydro-1,2,4-thiadiazol-3-yl)benzoic acid (470 mg, 65%) as a white solid, which was used in the next step directly without further purification. ¹H NMR (DMSO-d₆, 400 MHz) δ_{H}=8.57-8.52 (m, 1H), 8.21-8.14 (m, 1H), 8.13-8.06 (m, 1H), 7.66 (t, J=7.6 Hz, 1H).

### Synthesis of N-(5-(2-(2-aminopyridin-3-yl)-5-phenyl-3H-imidazo[4,5-b]pyridin-3-yl)-6-methylpyridin-2-yl)-3-(5-oxo-4,5-dihydro-1,2,4-thiadiazol-3-yl)benzamide (I-40)

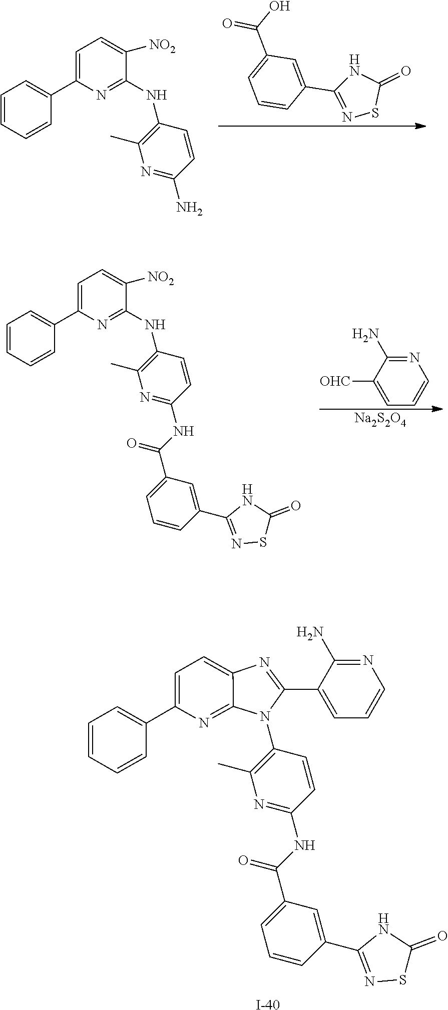

Step 1. To a solution of 6-methyl-N5-(3-nitro-6-phenylpyridin-2-yl)pyridine-2,5-diamine (500 mg, 1.56 mmol) and 3-(5-oxo-4,5-dihydro-1,2,4-thiadiazol-3-yl)benzoic acid (380 mg, 1.71 mmol) in pyridine (5.00 mL) was added EDCI (895 mg, 4.67 mmol) at 25° C. The mixture was stirred at 80° C. for 2 hrs. The mixture was diluted with EtOAc (100 mL) and washed with brine (30 mL×2). The organic layer was concentrated to give N-(6-methyl-5-((3-nitro-6-phenylpyridin-2-yl)amino)pyridin-2-yl)-3-(5-oxo-4,5-dihydro-1,2,4-thiadiazol-3-yl)benzamide (740 mg, 90% yield) as a yellow solid. LCMS Rₜ=1.668 min in 2.5 min chromatography, Agilent PoroShell 120 EC-C18 2.7 μm 3.0*50 mm, purity 39.7%, MS ESI calcd. for 525.12 [M+H]⁺ 526.12, found 526.2.

Step 2. To a solution of N-(6-methyl-5-((3-nitro-6-phenylpyridin-2-yl)amino)pyridin-2-yl)-3-(5-oxo-4,5-dihydro-1,2,4-thiadiazol-3-yl)benzamide (590 mg, 1.12 mmol) in DMSO (10.0 mL) and methanol (2.00 mL) was added 2-aminopyridine-3-carbaldehyde (274 mg, 2.25 mmol) and Na₂S₂O₄ (1.17 g, 6.74 mmol) at 25° C. The mixture was stirred at 100° C. for 10 hrs. The reaction was quenched with saturated aqueous NaHCO₃ (30 mL) drop-wise. The resulting mixture was extracted with EtOAc (50 mL×3), and the combined organic phase was dried over anhydrous Na₂SO₄, filtered and concentrated. The residue was purified by prep-HPLC (Column: F-Welch Xtimate C18 40*200 mm 7 μm; mobile phase: [water (0.05% HCl)-MeOH]; B %: 32%-72%; 60 min) and prep-HPLC (Column: YMC Triart 30*150 mm*7 μm; mobile phase: [water (NH₄HCO₃)-ACN]; B %: 10%-50%; 25 min) to give N-[5-[2-(2-amino-3-pyridyl)-5-phenyl-imidazo[4,5-b]pyridin-3-yl]-6-methyl-2-pyridyl]-3-(5-oxo-4H-1,2,4-thiadiazol-3-yl)benzamide I-40 (14 mg, 2% yield) as a yellow solid. 1H NMR (DMSO-d₆, 400 MHz) δ_{H}=11.13 (s, 1H), 8.72-8.56 (m, 1H), 8.33 (d, J=8.0 Hz, 1H), 8.28 (d, J=8.8 Hz, 1H), 8.19 (d, J=7.6 Hz, 2H), 8.06-7.97 (m, 5H), 7.73-7.64 (m, 1H), 7.51-7.45 (m, 2H), 7.43-7.38 (m, 1H), 7.25 (d, J=7.2 Hz, 1H), 7.22-7.14 (m, 1H), 6.52-6.46 (m, 1H), 2.15 (s, 3H). HPLC R₁=2.972 min in 8 min chromatography, 10-80AB, purity 99.8%. LCMS Rₜ=2.075 min in 4 min chromatography, Agilent PoroShell 120 EC-C18 2.7 μm 3.0*50 mm, purity 99.4%, MS ESI calcd. for 597.17 [M+H]⁺ 598.17, found 598.2.

### Example 33. Synthesis of N-(5-(2-(2-aminopyridin-3-yl)-5-phenyl-3H-imidazo[4,5-b]pyridin-3-yl)pyridin-2-yl)-2-fluoro-4-(5-oxo-4,5-dihydro-1,2,4-thiadiazol-3-yl)benzamide (I-41)

### Synthesis of 2-fluoro-4-(5-oxo-4,5-dihydro-1,2,4-thiadiazol-3-yl)benzoic acid

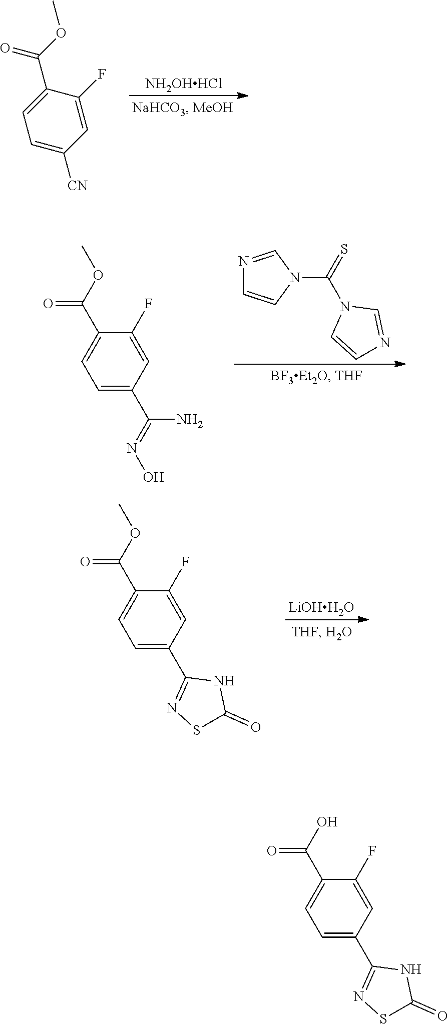

Step 1. To a solution of methyl 4-cyano-2-fluoro-benzoate (5.00 g, 27.9 mmol) in methanol (50 mL) was added NaHCO₃ (2.58 g, 30.7 mmol) and hydroxylamine hydrochloride (1.94 g, 27.9 mmol). The mixture was stirred at 70° C. for 3 hrs under N₂. Water was added, and the precipitate collected by filtration, washed and dried in vacuo to give methyl 2-fluoro-4-[(Z)—N′-hydroxycarbamimidoyl]benzoate (470 mg, 65% yield) as a white solid, which was used in the next step without further purification. ¹H NMR (400 MHz, DMSO-d₆) δ=10.05 (s, 1H), 7.88 (t, J=8.0 Hz, 1H), 7.68-7.55 (m, 2H), 6.00 (s, 2H), 3.86 (s, 3H).

Step 2. To a solution of methyl 2-fluoro-4-[(Z)—N′-hydroxycarbamimidoyl]benzoate (3.00 g, 15.4 mmol) in THF (30.0 mL) was added di(imidazol-1-yl)methanethione (4.13 g, 23.2 mmol). The mixture was stirred at 25° C. for 2 hrs under N₂. The mixture was poured into water and extracted with DCM (80 mL). The organic phase was washed with brine (10 mL), dried with Na₂SO₄ and concentrated. The residue was dissolved in THF (20 mL). To the mixture was added BF₃·Et₂O (14.0 mL, 46.3 mmol) dropwise. The mixture was stirred at 25° C. for 2 hrs under N₂. The mixture was diluted with water (50 mL), sat, aq. NaHCO₃ (50 mL), and the mixture was extracted with EtOAc (100 mL). The organic phase was washed with brine (30 mL), dried with Na₂SO₄ and concentrated to give methyl 2-fluoro-4-(5-oxo-4H-1,2,4-thiadiazol-3-yl)benzoate (4.00 g, crude) as a white solid, which was used in the next step without further purification. ¹H NMR (400 MHz, DMSO-d₆) δ=8.02 (br t, J=7.6 Hz, 1H), 7.95-7.82 (m, 2H), 3.88 (s, 3H).

Step 3. To a solution of methyl 2-fluoro-4-(5-oxo-4H-1,2,4-thiadiazol-3-yl)benzoate (3.00 g, 11.8 mmol) in THF (2.00 mL) was added LiOH·H₂O (1.49 g, 35.4 mmol) and water (1.00 mL). The mixture was stirred at 25° C. for 2 hrs. The reaction mixture was then adjusted to pH~3 by aq. HCl (1 N). The reaction mixture was filtered and the filter cake was concentrated to give 2-fluoro-4-(5-oxo-4H-1,2,4-thiadiazol-3-yl)benzoic acid (1.30 g, 46% yield) as a light yellow solid, which was used in the next step without further purification. ¹H NMR (400 MHz, DMSO-d₆) δ=8.03-7.96 (m, 1H), 7.90-7.81 (m, 2H).

### Synthesis of N-(5-(2-(2-aminopyridin-3-yl)-5-phenyl-3H-imidazo[4,5-b]pyridin-3-yl)pyridin-2-yl)-2-fluoro-4-(5-oxo-4,5-dihydro-1,2,4-thiadiazol-3-yl)benzamide (I-41)

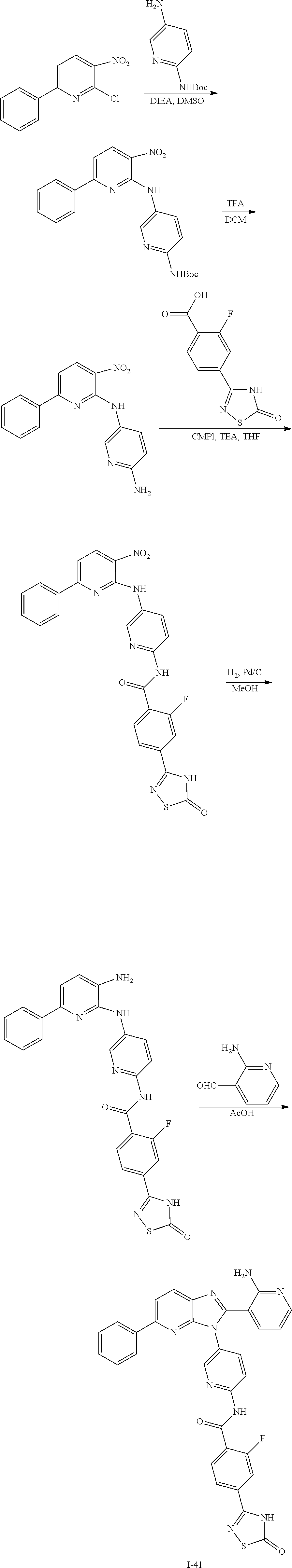

Step 1. To a solution of tert-butyl (5-aminopyridin-2-yl)carbamate (8.44 g, 40.3 mmol) in DMSO (90.0 mL) was added 2-chloro-3-nitro-6-phenylpyridine (8.60 g, 36.7 mmol) and DIEA (14.2 g, 110 mmol) at 25° C. The mixture was stirred at 80° C. for 14 hrs. The mixture was diluted with EtOAc (300 mL) and washed with brine (200 mL×2). The organic layer was dried over Na₂SO₄ and concentrated to give the crude product. The mixture was pulping with isopropyl ether (100 mL) for 10 mins. The reaction mixture was filtered, and the filter cake was concentrated to give tert-butyl (5-((3-nitro-6-phenylpyridin-2-yl)amino)pyridin-2-yl)carbamate (13.40 g, 90%) as a red solid, which was used in the next step directly. ¹H NMR (DMSO-d₆, 400 MHz) δ_{H}=10.10-10.04 (m, 1H), 9.81 (s, 1H), 8.64-8.55 (m, 2H), 8.09-8.00 (m, 3H), 7.85 (d, J=8.8 Hz, 1H), 7.59 (d, J=8.8 Hz, 1H), 7.55-7.49 (m, 3H), 1.50-1.46 (m, 9H).

Step 2. To a solution of tert-butyl (5-((3-nitro-6-phenylpyridin-2-yl)amino)pyridin-2-yl)carbamate (12.4 g, 30.4 mmol) in DCM (60.0 mL) was added TFA (60.0 mL, 30.4 mmol). The mixture was stirred at 25° C. for 10 hrs. The reaction mixture was concentrated. The reaction mixture was then adjusted to pH~7 by NaOH (2M). The resulting mixture was extracted with EtOAc (100 mL×3). The combined organic phase was dried over anhydrous Na₂SO₄, filtered and concentrated to give N5-(3-nitro-6-phenylpyridin-2-yl)pyridine-2,5-diamine (11.8 g, crude) as a white solid, which was used in the next step directly. ¹H NMR (DMSO-d₆, 400 MHz) δ_{H}=10.00 (s, 1H), 8.61 (d, J=8.8 Hz, 1H), 8.29 (d, J=2.4 Hz, 1H), 8.19 (dd, J=2.4, 9.2 Hz, 1H), 8.06-7.99 (m, 2H), 7.60 (d, J=8.8 Hz, 1H), 7.54-7.46 (m, 3H), 7.03 (d, J=9.6 Hz, 1H).

Step 3. To a solution of N5-(3-nitro-6-phenyl-2-pyridyl)pyridine-2,5-diamine (500 mg, 1.63 mmol) and 2-fluoro-4-(5-oxo-4H-1,2,4-thiadiazol-3-yl)benzoic acid (586 mg, 2.44 mmol) in THF (10.0 mL) was added TEA (2.20 mL, 16.2 mmol) and CMPI (499 mg, 1.95 mmol) at 25° C. The mixture was stirred at 65° C. for 12 hrs. Water (5 mL) was added to the reaction mixture, and the resulting mixture was extracted with DCM (15 mL×3). The combined organic phase was washed with brine (5 mL), dried over anhydrous Na₂SO₄, filtered and concentrated. The residue was purified by flash silica gel chromatography (eluent of 0-6% MeOH in DCM) to give 2-fluoro-N-[5-[(3-nitro-6-phenyl-2-pyridyl)amino]-2-pyridyl]-4-(5-oxo-4H-1,2,4-thiadiazol-3-yl)benzamide (90.0 mg, 26% yield) as a red solid. ¹H NMR (400 MHz, DMSO-d₆) δ=10.83 (br s, 1H), 10.15 (s, 1H), 8.80-8.59 (m, 2H), 8.35-8.04 (m, 4H), 7.90 (br d, J=7.2 Hz, 1H), 7.82-7.70 (m, 2H), 7.65-7.50 (m, 4H).

Step 4. To a solution of 2-fluoro-N-[5-[(3-nitro-6-phenyl-2-pyridyl)amino]-2-pyridyl]-4-(5-oxo-4H-1,2,4-thiadiazol-3-yl)benzamide (400 mg, 0.755 mmol) in methanol (10.0 mL) was added wet Pd/C (10%, 0.100 g) under N₂ atmosphere. The suspension was degassed and purged with H₂ (×3). The reaction mixture was stirred under H₂ (30 Psi) at 25° C. for 1 hr. The reaction mixture was filtered and the filtrate was concentrated to give N-[5-[(3-amino-6-phenyl-2-pyridyl)amino]-2-pyridyl]-2-fluoro-4-(5-oxo-4H-1,2,4-thiadiazol-3-yl)benzamide (150 mg, 40% yield) as a green solid, which was used in the next step without further purification. LCMS Rₜ=0.618 min in 1.0 min chromatography, purity 96.5%, MS ESI calcd. for 499.12 [M+H]⁺ 500.12, found 500.3.

Step 5. To a solution of N-[5-[(3-amino-6-phenyl-2-pyridyl)amino]-2-pyridyl]-2-fluoro-4-(5-oxo-4H-1,2,4-thiadiazol-3-yl)benzamide (150 mg, 0.300 mmol) in acetic acid (3.00 mL) was added 2-aminopyridine-3-carbaldehyde (73.0 mg, 0.601 mmol) under 02. The mixture was stirred at 80° C. for 1 hr. The reaction mixture was then adjusted to pH~7 by sat. aq. NaHCO₃, and the resulting mixture was extracted with EtOAc (15 mL×3). The combined organic phase was washed with brine (5 mL), dried over anhydrous Na₂SO₄, filtered and concentrated. The residue was purified by prep. HPLC (Column: Welch Xtimate C18 40*200 mm 7 um; mobile phase: [water (HCl)-ACN]; B %: 18%-58%, 30 min) to give N-[5-[2-(2-amino-3-pyridyl)-5-phenyl-imidazo[4,5-b]pyridin-3-yl]-2-pyridyl]-2-fluoro-4-(5-oxo-4H-1,2,4-thiadiazol-3-yl)benzamide I-41 (26.6 mg, 14% yield, HCl salt) as a yellow solid. ¹H NMR (400 MHz, DMSO-d₆) δ=13.67 (s, 1H), 11.35 (s, 1H), 8.62 (d, J=2.4 Hz, 1H), 8.39 (d, J=8.4 Hz, 3H), 8.18-8.08 (m, 5H), 8.04 (dd, J=0.8, 7.6 Hz, 1H), 7.94-7.85 (m, 3H), 7.53-7.41 (m, 3H), 7.01-6.94 (m, 1H). HPLC Rₜ=2.995 min in 8 min chromatography, purity 92.2%. LCMS Rₜ=1.360 min in 2.0 min chromatography, purity 92.0%, MS ESI calcd. for 601.14 [M+H]⁺ 602.14. found 602.0.

### Example 34. Synthesis of N-(5-(2-(2-aminopyridin-3-yl)-5-phenyl-3H-imidazo[4,5-b]pyridin-3-yl)-6-methylpyridin-2-yl)-4-(5-oxo-4,5-dihydro-1,2,4-thiadiazol-3-yl)benzamide (I-42)

### Synthesis of 4-(5-oxo-4,5-dihydro-1,2,4-thiadiazol-3-yl)benzoic acid

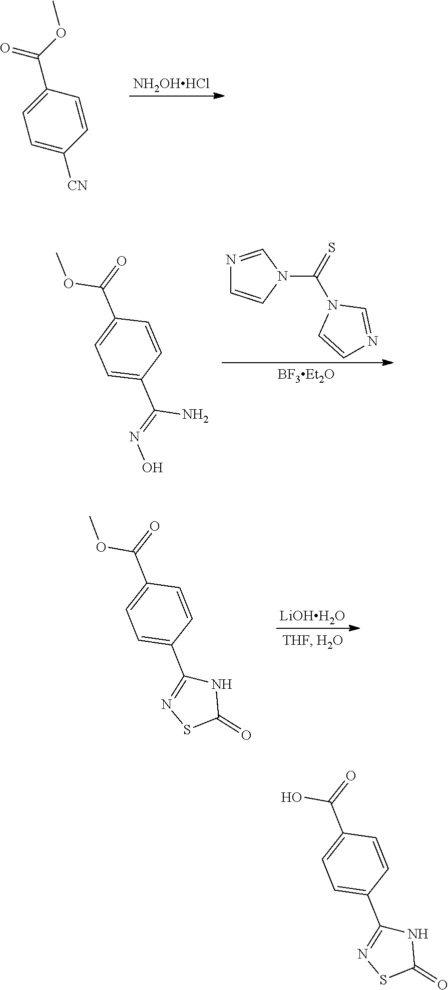

Step 1. A solution of methyl 4-cyanobenzoate (3.00 g, 18.6 mmol), hydroxylamine hydrochloride (1.29 g, 18.6 mmol) and NaHCO₃ (1.72 g, 20.5 mmol) in methanol (30.0 mL) was stirred at 25° C. for 0.5 hrs. The reaction mixture was then heated to 80° C. for 3 hrs under N₂ atmosphere. The mixture was diluted with H₂O (100 mL), extracted with EtOAc (100 mL×3), and washed with brine (50 mL). The organic layer was dried over Na₂SO₄ and concentrated to give methyl (Z)-4-(N′-hydroxycarbamimidoyl)benzoate (3.40 g, crude) as a light yellow solid, which was used in the next step without further purification. ¹H NMR (400 MHz, DMSO-d₆) δ=9.90 (s, 1H), 7.95 (d, J=8.4 Hz, 2H), 7.82 (d, J=8.4 Hz, 2H), 5.93 (s, 2H), 3.86 (s, 3H).

Step 2. To a solution of methyl 4-[(Z)—N′-hydroxycarbamimidoyl]benzoate (3.40 g, 17.2 mmol) in THF (50.0 mL) was added di(imidazol-1-yl)methanethione (4.59 g, 25.8 mmol). The mixture was stirred at 25° C. for 1 hr under N₂ atmosphere. The mixture was poured into water (100 mL) and extracted with DCM (100 mL×3). The organic phase was washed with brine (50 mL), dried with Na₂SO₄ and concentrated. The residue was dissolved in THF (50 mL). To the mixture was added BF₃·Et₂O (16.0 mL, 51.5 mmol) dropwise, and the mixture was stirred at 25° C. for 3 hrs under N₂ atmosphere. The reaction was quenched with saturated aqueous NaHCO₃ (200 mL) drop-wise. The resulting mixture was extracted with EtOAc (200 mL×3), and the combined organic phase was washed with brine (100 mL), dried over anhydrous Na₂SO₄, filtered and concentrated. The residue was purified by flash silica gel chromatography (eluent of 32% EtOAc in PE) to give methyl 4-(5-oxo-4H-1,2,4-thiadiazol-3-yl)benzoate (1.80 g, 40% yield) as a white solid. ¹H NMR (400 MHz, DMSO-d₆) δ=8.07 (s, 4H), 3.88 (s, 3H).

Step 3. To a solution of methyl 4-(5-oxo-4H-1,2,4-thiadiazol-3-yl)benzoate (1.80 g, 11.9 mmol) in THF (20.0 mL) and water (5.00 mL) was added LiOH·H₂O (959 mg, 22.9 mmol) at 25° C., and the reaction mixture was stirred for 1 hr. The reaction mixture was concentrated, the mixture was diluted with H₂O (30 mL), extracted with PE (10 mL×3), and the pH of the aqueous phase was adjusted to pH~2 with HCl (2 M). The resulting mixture was extracted with DCM (20 mL×3). The combined organic phase was dried over anhydrous Na₂SO₄, filtered and concentrated to give 4-(5-oxo-4H-1,2,4-thiadiazol-3-yl)benzoic acid (1.00 g, 38% yield) as a white solid, which was used in the next step without further purification. LCMS Rₜ=0.967 min in 2.5 min chromatography, purity 68.6%, MS ESI calcd. For 222.01 [M+H]⁺ 223.01, found 223.0.

### Synthesis of N-(5-(2-(2-aminopyridin-3-yl)-5-phenyl-3H-imidazo[4,5-b]pyridin-3-yl)-6-methylpyridin-2-yl)-4-(5-oxo-4,5-dihydro-1,2,4-thiadiazol-3-yl)benzamide (I-42)

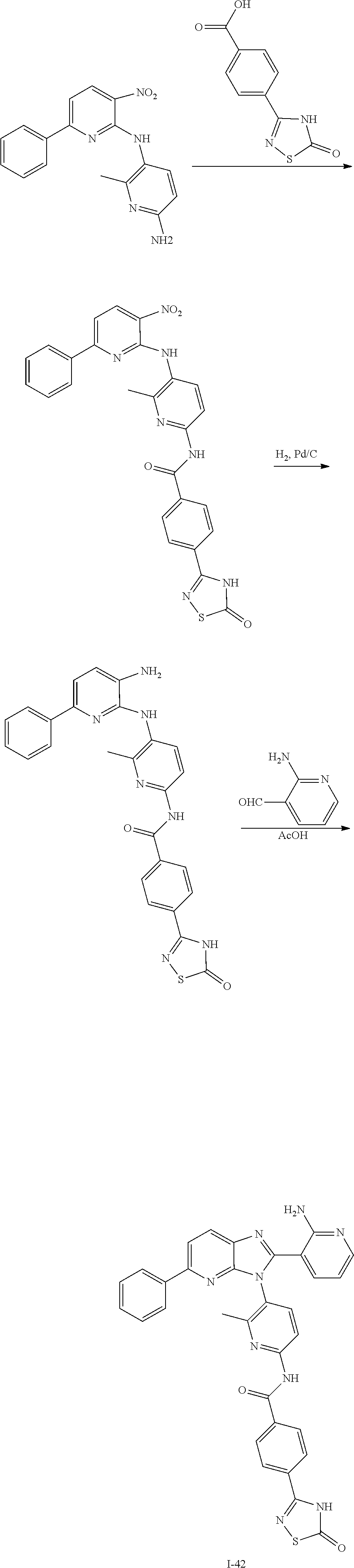

Step 1. To a solution of 6-methyl-N5-(3-nitro-6-phenyl-2-pyridyl)pyridine-2,5-diamine (400 mg, 1.24 mmol) in pyridine (5.00 mL) was added 4-(5-oxo-4H-1,2,4-thiadiazol-3-yl)benzoic acid (553 mg, 2.48 mmol) and EDCI (716 mg, 3.73 mmol). The reaction mixture was stirred at 25° C. for 12 hrs. The mixture was diluted with H₂O (30 mL), extracted with EtOAc (30 mL×3), and washed with brine (15 mL). The organic layer was dried over Na₂SO₄ and concentrated to give crude product. The residue was purified by flash silica gel chromatography (eluent of 12% MeOH in DCM gradient) to give N-[6-methyl-5-[(3-nitro-6-phenyl-2-pyridyl)amino]-2-pyridyl]-4-(5-oxo-4H-1,2,4-thiadiazol-3-yl)benzamide (135 mg, 21% yield) as a yellow solid. LCMS Rₜ=1.619 min in 2.5 min chromatography, purity 27.0%, MS ESI calcd. For 525.12 [M+H]⁺ 526.12, found 526.1.

Step 2. To a solution of N-[6-methyl-5-[(3-nitro-6-phenyl-2-pyridyl)amino]-2-pyridyl]-4-(5-oxo-4H-1,2,4-thiadiazol-3-yl)benzamide (135 mg, 0.260 mmol) in methanol (5.00 mL) was added wet Pd/C (50.0 mg, purity 10%) under N₂ atmosphere. The reaction mixture was stirred at 25° C. for 1 hr. The mixture was filtered via a celite pad and the filter cake was washed with DCM/MeOH=1/1 (200 mL). The organic phase was concentrated to give N-[5-[(3-amino-6-phenyl-2-pyridyl)amino]-6-methyl-2-pyridyl]-4-(5-oxo-4H-1,2,4-thiadiazol-3-yl)benzamide (51.7 mg, 41% yield) as a yellow solid, which was used in the next step without further purification. LCMS Rₜ=1.227 min in 2.5 min chromatography, purity 56.3%, MS ESI calcd. For 495.15 [M+H]⁺ 496.15, found 496.2.

Step 3. To a solution of N-[5-[(3-amino-6-phenyl-2-pyridyl)amino]-6-methyl-2-pyridyl]-4-(5-oxo-4H-1,2,4-thiadiazol-3-yl)benzamide (51.7 mg, 0.110 mmol) in acetic acid (5.00 mL) was added 2-aminopyridine-3-carbaldehyde (27.0 mg, 0.220 mmol) under O₂ atmosphere. The mixture was stirred at 80° C. for 1 hr under N₂ atmosphere. The reaction mixture was adjusted to pH~7 by saturated aq. NaHCO₃ (20 mL). The resulting mixture was extracted with EtOAc (15 mL×3). The combined organic phase was washed with brine (10 mL), dried over anhydrous Na₂SO₄, filtered and concentrated. The crude product was purified by prep-HPLC (column: Welch Xtimate C18 40*200 mm 7 μm; mobile phase: [water (FA)-ACN]; B %: 0%-20%, 30 min) to give N-(5-(2-(2-aminopyridin-3-yl)-5-phenyl-3H-imidazo[4,5-b]pyridin-3-yl)-6-methylpyridin-2-yl)-4-(5-oxo-4,5-dihydro-1,2,4-thiadiazol-3-yl)benzamide I-42 (8.50 mg, 19% yield) as a yellow solid. LCMS Rₜ=2.764 min in 4 min chromatography, purity 100%. MS ESI calcd. For 597.17 [M+H]⁺ 598.17, found 598.1. HPLC Rₜ=4.275 min in 8 min chromatography, purity 100%. ¹H NMR (400 MHz, DMSO-d₆) δ=13.63-13.50 (m, 1H), 11.30-11.23 (m, 1H), 8.37 (d, J=8.4 Hz, 1H), 8.26 (d, J=8.8 Hz, 1H), 8.22-8.17 (m, 2H), 8.11-8.02 (m, 6H), 8.01-7.97 (m, 1H), 7.90-7.59 (m, 1H), 7.48 (t, J=7.2 Hz, 3H), 7.45-7.39 (m, 1H), 6.71-6.64 (m, 1H), 2.21 (s, 3H).

### Example 35 and Example 36. Synthesis of 3-((1r,4r)-4-((3-(4-(2-(2-aminopyridin-3-yl)-5-phenyl-3H-imidazo[4,5-b]pyridin-3-yl)phenyl)azetidin-1-yl)methyl)cyclohexyl)-1,2,4-thiadiazol-5(4H)-one (I-43) and 3-((1s,4s)-4-((3-(4-(2-(2-aminopyridin-3-yl)-5-phenyl-3H-imidazo[4,5-b]pyridin-3-yl)phenyl)azetidin-1-yl)methyl)cyclohexyl)-1,2,4-thiadiazol-5(4H)-one (I-44)

### Synthesis of 4-formylcyclohexane-1-carbonitrile

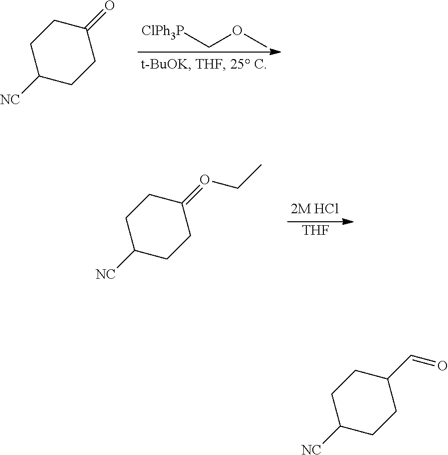

Step 1. To a solution of chloro(methoxymethyl)triphenyl-15-phosphane (2.09 g, 6.09 mmol) in THF (10 mL) was added t-BuOK (6.5 mL, 6.50 mmol) at 0° C. under N₂ atmosphere. The resulting mixture was stirred at 0° C. for 30 mins. A solution of 4-oxocyclohexane-1-carbonitrile (500 mg, 4.06 mmol) in THF (5 mL) was added into the reaction at 0° C. The resulting mixture was stirred at 25° C. for 2 hrs under N₂ atmosphere. The reaction was quenched with aqueous NH₄Cl (10 mL) drop-wise. The resulting mixture was extracted with DCM (20 mL×3). The combined organic phase was washed with brine (10 mL), dried over anhydrous Na₂SO₄, filtered and concentrated. The residue was purified by combi-flash column (EtOAc in PE=0%-7%) to afford 4-(methoxymethylene)cyclohexane-1-carbonitrile (500 mg, 3.31 mmol, 81% yield) as a colorless oil. ¹H NMR (400 MHz, CHLOROFORM-d) δ=5.80 (s, 1H), 3.54 (s, 3H), 2.74-2.68 (m, 1H), 2.55-2.46 (m, 1H), 2.22-2.07 (m, 2H), 2.00-1.91 (m, 1H), 1.91-1.81 (m, 2H), 1.78-1.67 (m, 2H).

Step 2. To a solution of 4-(methoxymethylene)cyclohexane-1-carbonitrile (500 mg, 3.31 mmol) in THF (3 mL) was added HCl (6.0 mL, 2 M in water) at 25° C. The resulting mixture was stirred at 25° C. for 12 hrs. Water (8 mL) was added to the reaction mixture. The resulting mixture was extracted with DCM (15 mL×3). The combined organic phase was washed with brine (10 mL), dried over anhydrous Na₂SO₄, filtered and concentrated to afford 4-formylcyclohexane-1-carbonitrile (500 mg, crude) as a yellow oil. ¹H NMR (400 MHz, CHLOROFORM-d) δ=9.62 (s, 1H), 2.93-2.45 (m, 1H), 2.38-2.23 (m, 1H), 2.13-2.03 (m, 2H), 2.01-1.76 (m, 3H), 1.75-1.59 (m, 2H), 1.53-1.38 (m, 1H).

### Synthesis of 3-((1r,4r)-4-((3-(4-(2-(2-aminopyridin-3-yl)-5-phenyl-3H-imidazo[4,5-b]pyridin-3-yl)phenyl)azetidin-1-yl)methyl)cyclohexyl)-1,2,4-thiadiazol-5(4H)-one (I-43) and 3-((1s,4s)-4-((3-(4-(2-(2-aminopyridin-3-yl)-5-phenyl-3H-imidazo[4,5-b]pyridin-3-yl)phenyl)azetidin-1-yl)methyl)cyclohexyl)-1,2,4-thiadiazol-5(4H)-one (I-44)

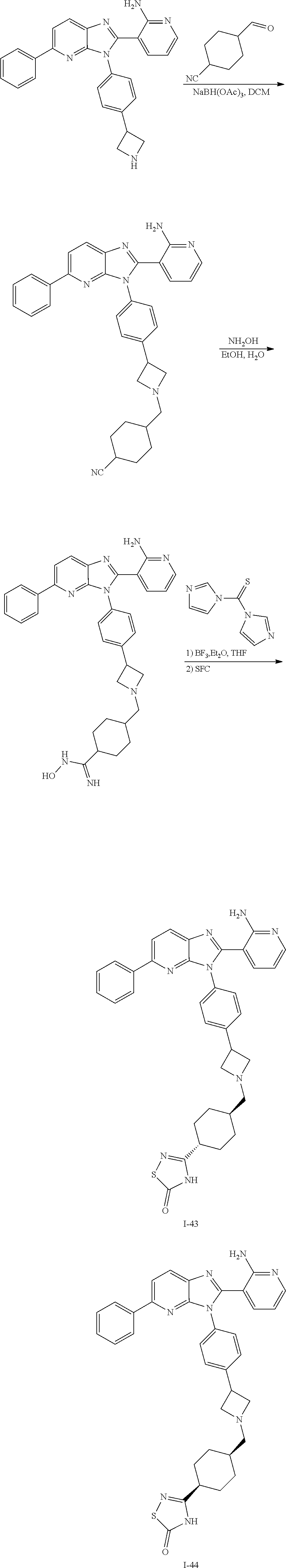

Step 1. To a solution of 4-formylcyclohexane-1-carbonitrile (200 mg, 1.46 mmol) and 3-(3-(4-(azetidin-3-yl)phenyl)-5-phenyl-3H-imidazo[4,5-b]pyridin-2-yl)pyridin-2-amine (610 mg, 1.46 mmol) in DCM (6 mL) was added TEA (443 mg, 4.37 mmol) at 25° C. The resulting mixture was stirred at 25° C. for 30 mins. NaBH(OAc)₃ (927 mg, 4.37 mmol) was added into the reaction and the reaction was stirred at 25° C. for 12 hrs under N₂ atmosphere. Water (5 mL) was added to the reaction mixture. The resulting mixture was extracted with DCM (10 mL×3). The combined organic phase was washed with brine (10 mL), dried over anhydrous Na₂SO₄, filtered and concentrated. The residue was purified by combi-flash column (MeOH in DCM=0%-4%) to afford 4-((3-(4-(2-(2-aminopyridin-3-yl)-5-phenyl-3H-imidazo[4,5-b]pyridin-3-yl)phenyl)azetidin-1-yl)methyl)cyclohexane-1-carbonitrile (420 mg, 0.778 mmol, 53% yield) as a yellow solid. ¹H NMR (400 MHz, CHLOROFORM-d) δ=8.14 (d, J=8.4 Hz, 1H), 8.05 (d, J=4.8 Hz, 1H), 8.00 (d, J=7.6 Hz, 2H), 7.81 (d, J=8.4 Hz, 1H), 7.51-7.36 (m, 7H), 7.12 (d, J=6.8 Hz, 1H), 6.87 (d, J=1.2 Hz, 2H), 6.44-6.35 (m, 1H), 4.42-4.14 (m, 2H), 3.94-3.50 (m, 2H), 3.05-2.68 (m, 3H), 2.40 (tt, J=3.6, 12.0 Hz, 1H), 2.20-2.14 (m, 1H), 2.07-1.78 (m, 4H), 1.70-1.54 (m, 3H), 1.50-1.39 (m, 1H).

Step 2. To a solution of 4-((3-(4-(2-(2-aminopyridin-3-yl)-5-phenyl-3H-imidazo[4,5-b]pyridin-3-yl)phenyl)azetidin-1-yl)methyl)cyclohexane-1-carbonitrile (430 mg, 0.800 mmol) in ethanol (5 mL) was added NH₂OH (1.11 g, 7.97 mmol, 50% in water) and NaHCO₃ (201 mg, 2.39 mmol) at 25° C. The resulting mixture was stirred at 90° C. for 12 hrs under N₂ atmosphere. Water (5 mL) was added into the reaction. The resulting mixture was extracted with DCM (10 mL×3). The combined organic phase was washed with brine (10 mL), dried over anhydrous Na₂SO₄, filtered and concentrated to afford 4-((3-(4-(2-(2-aminopyridin-3-yl)-5-phenyl-3H-imidazo[4,5-b]pyridin-3-yl)phenyl)azetidin-1-yl)methyl)-N-hydroxycyclohexane-1-carboximidamide (360 mg, crude) as a yellow oil. ¹H NMR (400 MHz, CHLOROFORM-d) δ=8.13 (d, J=8.4 Hz, 1H), 8.07 (dd, J=1.6, 4.8 Hz, 1H), 8.02 (d, J=7.6 Hz, 2H), 7.80 (d, J=8.4 Hz, 1H), 7.48-7.31 (m, 7H), 7.12 (dd, J=1.6, 8.0 Hz, 1H), 6.66 (s, 2H), 6.39 (dd, J=5.2, 7.6 Hz, 1H), 5.12-5.11 (m, 1H), 4.48 (d, J=7.2 Hz, 2H), 3.81 (s, 3H), 3.20 (s, 2H), 2.52 (d, J=6.8 Hz, 1H), 2.39 (d, J=6.8 Hz, 1H), 1.94 (t, J=11.2 Hz, 2H), 1.68-1.64 (m, 3H), 1.46-1.33 (m, 2H), 1.10-0.93 (m, 1H).

Step 3. To a solution of 4-((3-(4-(2-(2-aminopyridin-3-yl)-5-phenyl-3H-imidazo[4,5-b]pyridin-3-yl)phenyl)azetidin-1-yl)methyl)-N-hydroxycyclohexane-1-carboximidamide (365 mg, 0.640 mmol) in THF (2 mL) was added di(1H-imidazol-1-yl)methanethione (170 mg, 0.960 mmol) at 25° C. The resulting mixture was stirred at 25° C. for 1 hr under N₂ atmosphere. The mixture was poured into water (2 mL) and extracted with DCM (5 mL×3). The organic phase was washed with brine (5 mL), dried with Na₂SO₄ and concentrated. The residue was dissolved in THF (3 mL). Then BF₃·Et₂O (0.59 mL, 1.91 mmol) was added into the mixture at 0° C. under N₂ atmosphere. The mixture was stirred at 25° C. for 3 hrs under N₂ atmosphere. Water (10 mL) was added to the reaction mixture. The reaction mixture was then adjusted to pH~7 by saturated aqueous NaHCO₃. The resulting mixture was extracted with DCM (20 mL×3). The combined organic phase was washed with brine (20 mL), dried over anhydrous Na₂SO₄, filtered and concentrated. The residue was purified by prep-HPLC (column: F-Prepulite XP tC 18 40*200 mm*7 um; mobile phase: [water (NH₄HCO₃)-ACN]; B %: 24%-64%, 14 min) and further purified by prep-HPLC (column: Welch Xtimate C18 40*200 mm 7 um; mobile phase: [water (FA)-ACN]; B %: 0%-36%, 15 min), 3-(4-((3-(4-(2-(2-aminopyridin-3-yl)-5-phenyl-3H-imidazo[4,5-b]pyridin-3-yl)phenyl)azetidin-1-yl)methyl)cyclohexyl)-1,2,4-thiadiazol-5(4H)-one (70.0 mg, 0.122 mmol) was purified by prep-SFC (column: DAICEL CHIRALPAK AD 250 mm*30 mm, 10 um; mobile phase: CO₂-EtOH (0.1% NH₃H₂O); B %: 60%-60%, 80 min) to give 3-((1r,4r)-4-((3-(4-(2-(2-aminopyridin-3-yl)-5-phenyl-3H-imidazo[4,5-b]pyridin-3-yl)phenyl)azetidin-1-yl)methyl)cyclohexyl)-1,2,4-thiadiazol-5(4H)-one I-43 (35.0 mg, 0.0566 mmol, 46% yield) as a white solid and 3-((1s,4s)-4-((3-(4-(2-(2-aminopyridin-3-yl)-5-phenyl-3H-imidazo[4,5-b]pyridin-3-yl)phenyl)azetidin-1-yl)methyl)cyclohexyl)-1,2,4-thiadiazol-5(4H)-one I-44 (22.0 mg, 0.0350 mmol, 29% yield) as a white solid.

I-43: ¹H NMR (400 MHz, CDCl₃) δ=8.52 (s, 1H), 8.20 (d, J=8.4 Hz, 1H), 8.07-7.91 (m, 4H), 7.60-7.48 (m, 4H), 7.46-7.33 (m, 4H), 6.49 (dd, J=5.2, 7.6 Hz, 1H), 4.29-4.22 (m, 2H), 4.17-4.09 (m, 1H), 3.94-3.85 (m, 2H), 2.90 (d, J=6.8 Hz, 2H), 2.60-2.50 (m, 1H), 2.03 (d, J=13.6 Hz, 2H), 1.93 (d, J=9.6 Hz, 2H), 1.66-1.51 (m, 3H), 1.21-1.07 (m, 2H). HPLC Rₜ=3.168 min in 8 min chromatography, purity 98.2%. LCMS Rₜ=1.537 min in 4 min chromatography, purity 97.2%, MS ESI calcd. for 614.26 [M+H]⁺ 615.26, found 615.3.

I-44: ¹H NMR (400 MHz, CDCl₃) δ=8.19 (d, J=8.4 Hz, 1H), 8.03 (d, J=7.2 Hz, 2H), 7.99 (dd, J=1.6, 5.2 Hz, 1H), 7.95 (d, J=8.4 Hz, 1H), 7.57-7.51 (m, 2H), 7.51-7.46 (m, 2H), 7.46-7.33 (m, 4H), 6.50 (dd, J=5.2, 7.6 Hz, 1H), 4.15-4.07 (m, 2H), 4.06-3.97 (m, 1H), 3.74-3.62 (m, 2H), 2.85 (d, J=7.2 Hz, 2H), 2.78-2.68 (m, 1H), 2.03-1.93 (m, 2H), 1.83-1.78 (m, 1H), 1.77-1.63 (m, 4H), 1.61-1.52 (m, 2H). HPLC Rₜ=3.115 min in 8 min chromatography, purity 98.7%. LCMS Rₜ=1.557 min in 4 min chromatography, purity 97.4%, MS ESI calcd. for 614.26 [M+H]⁺ 615.26. found 615.3.

### Example 37 and Example 38. Synthesis of 3-((1r,4r)-4-((4-(4-(2-(2-aminopyridin-3-yl)-5-phenyl-3H-imidazo[4,5-b]pyridin-3-yl)phenyl)piperidin-1-yl)methyl)cyclohexyl)-1,2,4-thiadiazol-5(4H)-one (I-45) and 3-((1s,4s)-4-((4-(4-(2-(2-aminopyridin-3-yl)-5-phenyl-3H-imidazo[4,5-b]pyridin-3-yl)phenyl)piperidin-1-yl)methyl)cyclohexyl)-1,2,4-thiadiazol-5(4H)-one (I-46)

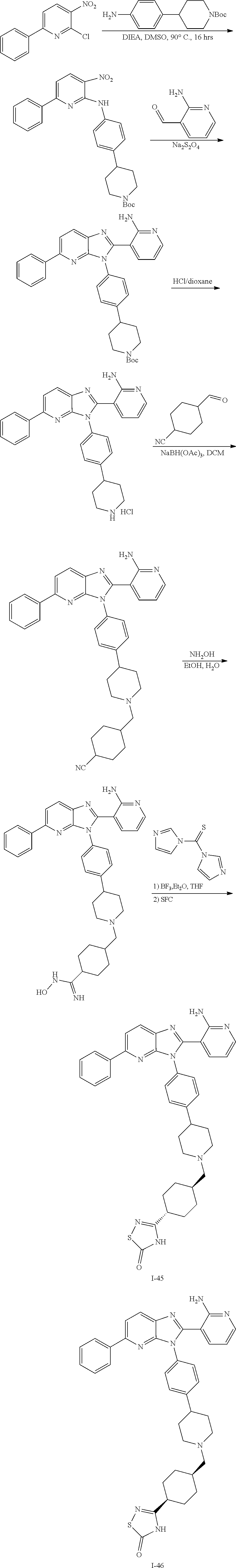

Step 1. To a solution of tert-butyl 4-(4-aminophenyl)piperidine-1-carboxylate (2.83 g, 10.2 mmol) in DMSO (20 mL) was added DIEA (3.30 g, 25.6 mmol) and 2-chloro-3-nitro-6-phenyl-pyridine (2.00 g, 8.52 mmol) at 25° C. The mixture was stirred at 80° C. for 14 hrs. EA (50 mL) was added to the reaction. The resulting suspension was washed with brine (40 mL×3), dried over anhydrous Na₂SO₄, filtered and concentrated. The mixture was triturated with MeCN (50 mL). The reaction mixture was filtered. The filter cake was concentrated to give tert-butyl 4-[4-[(3-nitro-6-phenyl-2-pyridyl)amino]phenyl]piperidine-1-carboxylate (3.30 g, 6.95 mmol, 82% yield) as a red solid. LCMS Rₜ=1.877 min in 2 min chromatography, purity 62.7%. MS ESI calcd. for 474.23, [M+H]⁺ 475.23. found 475.3.

Step 2. To a solution of tert-butyl 4-[4-[(3-nitro-6-phenyl-2-pyridyl)amino]phenyl]piperidine-1-carboxylate (2.80 g, 5.90 mmol) and 2-aminopyridine-3-carbaldehyde (1.44 g, 11.8 mmol) in DMSO (28 mL) and methanol (2 mL) was added Na₂S₂O₄ (5.96 g, 34.2 mmol) under N₂. The mixture was stirred at 100° C. for 12 hrs. The reaction mixture was concentrated. Water (40 mL) was added to the residue. The resulting mixture was extracted with EtOAc (60 mL×3). The combined organic phase was washed with brine (50 mL), dried over anhydrous Na₂SO₄, filtered and concentrated. The residue was purified by flash silica gel chromatography (0-52% of EtOAc in PE) to give tert-butyl 4-[4-[2-(2-amino-3-pyridyl)-5-phenyl-imidazo[4,5-b]pyridin-3-yl]phenyl]piperidine-1-carboxylate (1.23 g, 2.25 mmol, 38% yield) as a yellow solid. LCMS Rₜ=0.885 min in 1.5 min chromatography, purity 25.7%, MS ESI calcd. for 546.27, [M+H]⁺ 547.27, found 547.7.

Step 3. To a solution of tert-butyl 4-[4-[2-(2-amino-3-pyridyl)-5-phenyl-imidazo[4,5-b]pyridin-3-yl]phenyl]piperidine-1-carboxylate (0.40 g, 0.732 mmol) in 1,4-Dioxane (2 mL) was added HCl/dioxane (26 mL, 52.2 mmol). The mixture was stirred at 25° C. for 12 hrs. The reaction mixture was concentrated to give 3-[5-phenyl-3-[4-(4-piperidyl)phenyl]imidazo[4,5-b]pyridin-2-yl]pyridin-2-amine (500 mg, 1.12 mmol, HCl salt) as a yellow solid. LCMS Rₜ=0.618 min in 2.0 min chromatography, purity 94.6%, MS ESI calcd. for 446.22. [M+H]⁺ 447.22, found 447.2.

Step 4. To a solution of 3-[5-phenyl-3-[4-(4-piperidyl)phenyl]imidazo[4,5-b]pyridin-2-yl]pyridin-2-amine;hydrochloride (2.00 g, 4.14 mmol) and 4-formylcyclohexanecarbonitrile (568 mg, 4.14 mmol) in DCM (6 mL) was added TEA (1.70 mL, 12.4 mmol) at 25° C. The resulting mixture was stirred at 25° C. for 30 mins. NaBH(OAc)₃ (2.60 g, 12.4 mmol) was added. The reaction was stirred at 25° C. for 12 hrs. Water (50 mL) was added to the reaction mixture. The resulting mixture was extracted with DCM (100 mL×3). The combined organic phase was washed with brine (100 mL), dried over anhydrous Na₂SO₄, filtered and concentrated. The residue was purified by combi-flash column (MeOH in DCM=0%-4%) to give 4-[[4-[4-[2-(2-amino-3-pyridyl)-5-phenyl-imidazo[4,5-b]pyridin-3-yl]phenyl]-1-piperidyl]methyl]cyclohexanecarbonitrile (2.10 g, 3.70 mmol, 89% yield) as a yellow solid. 100 mg of this product was purified by prep-HPLC (column: F-Prepulite XP tC 18 40*200 mm*7 um; mobile phase: [water (NH₄HCO₃)-ACN]; B %: 56%-96%, 14 min to provide 4-[[4-[4-[2-(2-amino-3-pyridyl)-5-phenyl-imidazo[4,5-b]pyridin-3-yl]phenyl]-1-piperidyl]methyl]cyclohexanecarbonitrile (32.0 mg, 0.0545 mmol, 31% yield) as a white solid. ¹H NMR (400 MHz, DMSO-d₆) δ=8.27 (d, J=8.4 Hz, 1H), 8.07-7.94 (m, 4H), 7.54-7.36 (m, 7H), 7.17 (dd, J=1.6, 7.6 Hz, 1H), 6.97 (s, 2H), 6.40 (dd, J=4.8, 7.6 Hz, 1H), 2.95 (t, J=10.0 Hz, 2H), 2.65-2.56 (m, 2H), 2.22-2.07 (m, 2H), 2.05-1.93 (m, 3H), 1.89-1.42 (m, 10H), 1.19-1.05 (m, 1H), 0.99-0.82 (m, 1H). HPLC Rₜ=3.348 min in 4 min chromatography, ACE Excel 3 C18 4.6*100 mm, purity 97.8%. LCMS Rₜ=1.543 min in 4 min chromatography, Xtimate C18 2.1*30 mm, 3 μm, purity 98.7%, MS ESI calcd. for 567.31 [M+H]⁺ 568.31. found 568.4.

Step 5. To a solution of 4-[[4-[4-[2-(2-amino-3-pyridyl)-5-phenyl-imidazo[4,5-b]pyridin-3-yl]phenyl]-1-piperidyl]methyl]cyclohexanecarbonitrile (700 mg, 1.23 mmol) in ethanol (10 mL) was added NH₂OH (1.71 g, 12.3 mmol, 50% in water) and NaHCO₃ (311 mg, 3.70 mmol) at 25° C. The resulting mixture was stirred at 90° C. for 12 hrs under N₂ atmosphere. Water (50 mL) was added into the reaction. The resulting mixture was extracted with DCM (50 mL×3). The combined organic phase was washed with brine (30 mL), dried over anhydrous Na₂SO₄, filtered and concentrated to give 4-[[4-[4-[2-(2-amino-3-pyridyl)-5-phenyl-imidazo[4,5-b]pyridin-3-yl]phenyl]-1-piperidyl]methyl]-N-hydroxy-cyclohexanecarboxamidine (400 mg, 0.666 mmol, 54% yield) as a yellow oil. ¹H NMR (400 MHz, DMSO-d₆) δ=8.62 (d, J=10.4 Hz, 1H), 8.19 (d, J=8.4 Hz, 1H), 7.98-7.87 (m, 4H), 7.45-7.29 (m, 7H), 7.10 (dd, J=2.0, 7.6 Hz, 1H), 6.89 (s, 2H), 6.33 (dd, J=4.8, 7.6 Hz, 1H), 5.16 (d, J=12.8 Hz, 2H), 3.41-3.34 (m, 1H), 2.89 (dd, J=11.6, 16.0 Hz, 2H), 2.20 (d, J=7.6 Hz, 1H), 2.05 (d, J=7.2 Hz, 1H), 1.97-1.82 (m, 3H), 1.78-1.34 (m, 12H), 0.85-0.74 (m, 1H).

Step 6. To a solution of 4-[[4-[4-[2-(2-amino-3-pyridyl)-5-phenyl-imidazo[4,5-b]pyridin-3-yl]phenyl]-1-piperidyl]methyl]-N-hydroxy-cyclohexanecarboxamidine (400 mg, 0.666 mmol) in THF (2 mL) was added di(imidazol-1-yl)methanethione (178 mg, 0.999 mmol) at 25° C. The resulting mixture was stirred at 25° C. for 1 hr under N₂ atmosphere. The mixture was poured into water (2 mL) and extracted with DCM (5 mL×3). The organic phase was washed with brine (5 mL), dried with Na₂SO₄ and concentrated. The residue was dissolved in THF (3 mL). To the mixture was added BF₃·Et₂O (0.62 mL, 2.00 mmol) dropwise. The mixture was stirred at 25° C. for 3 hrs under N₂. The reaction mixture was stirred at 25° C. for 12 hrs. Water (50 mL) was added to the reaction mixture. The reaction mixture was then adjusted to pH~7 by saturated aqueous NaHCO₃. The resulting mixture was extracted with DCM (50 mL×3). The combined organic phase was washed with brine (50 mL), dried over anhydrous Na₂SO₄, filtered and concentrated. The crude product was purified by prep-HPLC (column: Welch Xtimate C18 40*200 mm 7 um; mobile phase: [water (FA)-ACN]; B %: 0%-38%, 15 min) and further purified by prep-HPLC (column: Welch Xtimate C18 40*200 mm 7 um; mobile phase: [water (TFA)-ACN]; B %: 6%-46%, 20 min) to give 3-[4-[[4-[4-[2-(2-amino-3-pyridyl)-5-phenyl-imidazo[4,5-b]pyridin-3-yl]phenyl]-1-piperidyl]methyl]cyclohexyl]-4H-1,2,4-thiadiazol-5-one (60 mg, mixture) as a light yellow solid. The mixture was then purified by SFC (column: DAICEL CHIRALCEL OX (250 mm*30 mm, 10 um); mobile phase: [CO₂-ACN/EtOH (0.1% NH₃H₂O)]; B %: 80%-80% to give 3-((1s,4s)-4-((4-(4-(2-(2-aminopyridin-3-yl)-5-phenyl-3H-imidazo[4,5-b]pyridin-3-yl)phenyl)piperidin-1-yl)methyl)cyclohexyl)-1,2,4-thiadiazol-5(4H)-one I-46 (14.0 mg, 0.0210 mmol, 10% yield) as a light yellow solid and 3-((1r,4r)-4-((4-(4-(2-(2-aminopyridin-3-yl)-5-phenyl-3H-imidazo[4,5-b]pyridin-3-yl)phenyl)piperidin-1-yl)methyl)cyclohexyl)-1,2,4-thiadiazol-5(4H)-one I-45 (16.0 mg, 0.0255 mmol, 13% yield) as a light yellow solid. I-45: ¹H NMR (400 MHz, DMSO-d₆) δ=8.27 (d, J=8.4 Hz, 1H), 8.06-7.94 (m, 4H), 7.54-7.33 (m, 7H), 7.17 (dd, J=1.6, 7.6 Hz, 1H), 6.97 (s, 2H), 6.40 (dd, J=4.8, 7.6 Hz, 1H), 2.97 (d, J=10.8 Hz, 2H), 2.61 (t, J=3.6 Hz, 1H), 2.46 (d, J=3.6 Hz, 1H), 2.17 (d, J=7.2 Hz, 2H), 2.10-1.77 (m, 9H), 1.76-1.65 (m, 2H), 1.58-1.39 (m, 3H), 1.01-0.87 (m, 2H). HPLC Rₜ=4.601 min in 8 min chromatography, ACE Excel 3 C18 4.6*100 mm, purity 99.8%. LCMS Rₜ=2.132 min in 4 min chromatography, Xtimate C18 2.1*30 mm, 3 μm, purity 99.9%, MS ESI calcd. for 642.83 [M+H]⁺ 643.83, found 643.4. I-46: ¹H NMR (400 MHz, DMSO-d₆) δ=8.27 (d, J=8.4 Hz, 1H), 8.06-7.96 (m, 4H), 7.51-7.38 (m, 7H), 7.17 (dd, J=1.8, 7.6 Hz, 1H), 6.97 (s, 2H), 6.40 (dd, J=4.8, 7.6 Hz, 1H), 2.99 (d, J=10.8 Hz, 2H), 2.68-2.56 (m, 2H), 2.27 (d, 0.1=7.6 Hz, 2H), 2.03 (t, 0.1=11.2 Hz, 2H), 1.87-1.42 (m, 14H). HPLC Rₜ=4.559 min in 8 min chromatography, ACE Excel 3 C18 4.6*100 mm, purity 99.55%. LCMS Rₜ=2.134 min in 4 min chromatography, Xtimate C18 2.1*30 mm, 3 μm, purity 99.73%. MS ESI calcd. for 642.29 [M+H]⁺ 643.29, found 643.4.

### Example 39 and Example 40. Synthesis of 3-((1r,4r)-4-((4-(2-(2-aminopyridin-3-yl)-5-phenyl-3H-imidazo[4,5-b]pyridin-3-yl)benzyl)(methyl)amino)cyclohexyl)-1,2,4-thiadiazol-5(4H)-one (I-47) and 3-((1s,4s)-4-((4-(2-(2-aminopyridin-3-yl)-5-phenyl-3H-imidazo[4,5-b]pyridin-3-yl)benzyl)(methyl)amino)cyclohexyl)-1,2,4-thiadiazol-5(4H)-one (I-48)

### Synthesis of (1r,4r)-4-((4-aminobenzyl)(methyl)amino)cyclohexane-1-carbonitrile

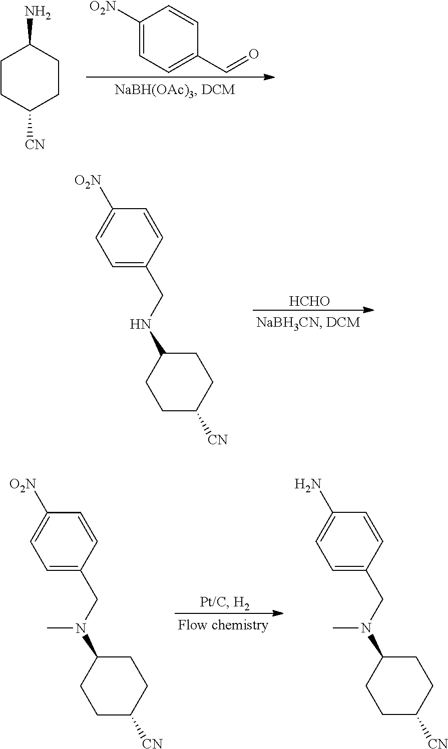

Step 1. To a solution of 4-nitrobenzaldehyde (2.43 g, 16.1 mmol) in DCM (30 mL) was added TEA (3.0 mL, 48.3 mmol) and (1r,4r)-4-aminocyclohexane-1-carbonitrile (2.00 g, 16.1 mmol). The mixture was stirred at 25° C. for 1 hr. Then the mixture was added NaBH(OAc)₃ (6.83 g, 32.2 mmol) at 25° C. The mixture was stirred at 25° C. for 16 hrs. Water (30 mL) was added to the residue. The resulting mixture was extracted with DCM (10 mL×3). The combined organic phase was washed with brine (20 mL), dried over anhydrous Na₂SO₄, filtered and concentrated. The residue was purified by flash silica gel chromatography (0-2% of MeOH in DCM) to afford (1r,4r)-4-((4-nitrobenzyl)amino)cyclohexane-1-carbonitrile (3.00 g, 11.6 mmol) as a yellow solid, ¹H NMR (DMSO-d₆, 400 MHz) δ=8.21-8.12 (m, 2H), 7.61 (d, J=8.4 Hz, 2H), 3.83 (s, 2H), 2.70-2.57 (m, 1H), 2.45-2.35 (m, 1H), 2.03-1.95 (m, 2H), 1.90-1.82 (m, 2H), 1.51-1.39 (m, 2H), 1.19-1.05 (m, 2H).

Step 2. To a solution of (1r,4r)-4-((4-nitrobenzyl)amino)cyclohexane-1-carbonitrile (3.00 g, 11.6 mmol) in methanol (30 mL) was added formaldehyde (1.88 g, 23.1 mmol). The mixture was stirred at 25° C. for 0.5 hr. NaBH₃CN (1.45 g, 23.1 mmol) was then added into the mixture. The mixture was stirred at 25° C. for 12 hrs. The reaction mixture was concentrated. Water (10 mL) was added to the reaction mixture. The resulting mixture was extracted with DCM (10 mL×3). The combined organic phase was washed with brine (10 mL), dried over anhydrous Na₂SO₄, filtered and concentrated. Water (10 mL) was added to the reaction mixture. The resulting mixture was extracted with DCM (10 mL×3). The combined organic phase was washed with brine (10 mL), dried over anhydrous Na₂SO₄, filtered and concentrated. The residue was purified by flash silica gel chromatography (0-30% EtOAc in PE) to afford (1r,4r)-4-(methyl(4-nitrobenzyl) amino)cyclohexane-1-carbonitrile (2.75 g, 10.1 mmol) as a yellow oil. ¹H NMR (DMSO-d₆, 400 MHz) δ=8.28-8.09 (m, 2H), 7.61-7.54 (m, 2H), 3.65 (s, 2H), 2.65-2.56 (m, 1H), 2.49-2.42 (m, 1H), 2.11-2.03 (m, 5H), 1.88-1.76 (m, 2H), 1.55-1.44 (m, 2H), 1.41-1.29 (m, 2H).

Step 3. Solution 1: (1r,4r)-4-(methyl(4-nitrobenzyl)amino)cyclohexane-1-carbonitrile (1.00 g, 3.66 mmol) in MeOH (20 mL). The fixed bed (named FLR1, volume 5 mL) was completely packed with granular catalyst 1% Pt/C (2 g). The H₂ back pressure regulator was adjusted to 1.0 MPa, and the flow rate of H₂ was 30 mL/min. Then the solution S1 was pumped by Pump 1 {S1,P1,0.303 mL/min} to fixed bed {FLRT,SS,Fixed bed, 6.350 (¼″) mm, 1 mL, 40° C.}. The solution S1 was flowing through {FLR1, 3.3 min} to leave the reactor zone, then the reaction mixture was collected from the reactor output. The reaction mixture was concentrated directly. The residue was purified by flash silica gel chromatography (eluent of 0-8% MeOH in DCM) to afford (1r,4r)-4-((4-aminobenzyl)(methyl)amino)cyclohexane-1-carbonitrile (200 mg, 0.822 mmol) as a yellow oil. ¹H NMR (DMSO-d₆, 400 MHz) δ=6.89 (d, J=8.4 Hz, 2H), 6.48 (d, J=8.4 Hz, 2H), 4.89 (s, 2H), 3.32-3.32 (m, 2H), 2.65-2.55 (m, 1H), 2.44-2.36 (m, 1H), 2.08-2.01 (m, 5H), 1.80-1.73 (m, 2H), 1.51-1.40 (m, 2H), 1.38-1.26 (m, 2H).

### Synthesis of 3-((1r,4r)-4-((4-(2-(2-aminopyridin-3-yl)-5-phenyl-3H-imidazo[4,5-b]pyridin-3-yl)benzyl)(methyl)amino)cyclohexyl)-1,2,4-thiadiazol-5(4H)-one (I-47) and 3-((1s,4s)-4-((4-(2-(2-aminopyridin-3-yl)-5-phenyl-3H-imidazo[4,5-b]pyridin-3-yl)benzyl)(methyl)amino)cyclohexyl)-1,2,4-thiadiazol-5(4H)-one (I-48)

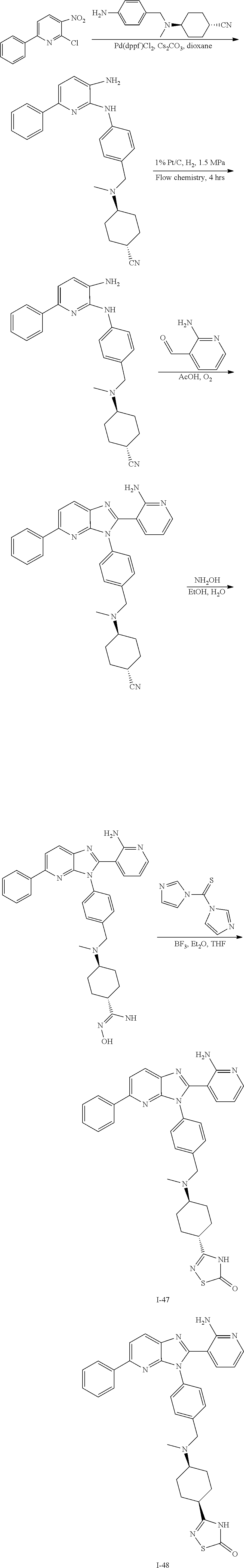

Step 1. To a solution of 2-chloro-3-nitro-6-phenylpyridine (1.10 g, 4.69 mmol) and (1r,4r)-4-((4-aminobenzyl)(methyl)amino)cyclohexane-1-carbonitrile (1.20 g, 4.92 mmol) in 1,4-Dioxane (40 mL) was added Cs₂CO₃ (4.58 g, 14.1 mmol) and Pd(dppf)C₁₂ (170 mg, 0.234 mmol). The reaction mixture was degassed and purged with N₂ for 3 times. The mixture was stirred at 90° C. for 8 hrs under N₂. The reaction mixture was concentrated. Water (10 mL) was added to the residue. The resulting mixture was extracted with EA (10 mL×3). The combined organic phase was washed with brine (10 mL), dried over anhydrous Na₂SO₄, filtered and concentrated. The residue was purified by flash silica gel chromatography (0-35% EA in PE) to afford (1r,4r)-4-(methyl(4-((3-nitro-6-phenylpyridin-2-yl)amino)benzyl)amino)cyclohexane-1-carbonitrile (1.60 g, 3.62 mmol) as a yellow oil, ¹H NMR (DMSO-d₆, 400 MHz) δ=10.10 (s, 1H), 8.60 (d, J=8.4 Hz, 1H), 8.13-8.04 (m, 2H), 7.68 (d, J=8.4 Hz, 2H), 7.60-7.50 (m, 4H), 7.33 (d, J=8.4 Hz, 2H), 3.54 (s, 2H), 2.67-2.58 (m, 1H), 2.48-2.43 (m, 1H), 2.13-2.05 (m, 5H), 1.88-1.79 (m, 2H), 1.54-1.44 (m, 2H), 1.42-1.33 (m, 2H).

Step 2. Solution 1: (1r,4r)-4-(methyl(4-((3-nitro-6-phenylpyridin-2-yl)amino)benzyl)amino) cyclohexane-1-carbonitrile (1.60 g, 3.62 mmol) in THF (80 mL). The fixed bed (named FLR1, volume 5 mL) was completely packed with granular catalyst 1% Pt/C (2.5 g). The H₂ back pressure regulator was adjusted to 1.5 MPa, and the flow rate of H₂ was 30 mL/min. Then the solution S1 was pumped by Pump 1 {S1, P1, 0.303 mL/min} to fixed bed {FLR1, SS, Fixed bed, 6.350 (¼″) mm, 1 mL, 40° C.}. The solution S1 was flowing through {FLR1, 3.3 min} to leave the reactor zone, then the reaction mixture was collected from the reactor output. The reaction mixture was concentrated directly to give (1r,4r)-4-((4-((3-amino-6-phenylpyridin-2-yl)amino)benzyl) (methyl)amino)cyclohexane-1-carbonitrile (1.10 g, crude) as a yellow oil, which was used for the next step directly. ¹H NMR (DMSO-d₆, 400 MHz) δ=7.91 (d, J=7.6 Hz, 2H), 7.82 (s, 1H), 7.71 (d, J=8.4 Hz, 2H), 7.39 (t, J=7.6 Hz, 2H), 7.27-7.17 (m, 4H), 6.96 (d, J=8.0 Hz, 1H), 5.25 (s, 2H), 3.46 (s, 2H), 2.65-2.57 (m, 1H), 2.48-2.42 (m, 1H), 2.10-2.04 (m, 5H), 1.85-1.79 (m, 2H), 1.54-1.36 (m, 4H).

Step 3. To a solution of (1r,4r)-4-((4-((3-amino-6-phenylpyridin-2-yl)amino)benzyl)(methyl) amino)cyclohexane-1-carbonitrile (100 mg, 0.243 mmol) in acetic acid (3 mL) was added 2-aminonicotinaldehyde (36.0 mg, 0.292 mmol). The reaction mixture was degassed and purged with 02 for 3 times. The mixture was stirred at 80° C. for 2 hrs under 02. The reaction mixture was concentrated directly. The residue was purified by flash silica gel chromatography (0-65% EA in PE). The residue was purified by prep-HPLC (Column: Welch Xtimate C18 150*30 mm*5 um; mobile phase: [water (NH₄HCO₃)-ACN]; B %: 0%-35%; 25 min) to afford (1r,4r)-4-((4-(2-(2-aminopyridin-3-yl)-5-phenyl-3H-imidazo [4,5-b]pyridin-3-yl) benzyl) (methyl)amino)cyclohexane-1-carbonitrile (65.0 mg, 0.126 mmol, 52% yield) as a yellow oil. ¹H NMR (DMSO-d₆, 400 MHz) δ=8.26 (d, J=8.4 Hz, 1H), 8.05-7.96 (m, 4H), 7.49-7.37 (m, 7H), 7.16 (dd, J=1.6, 7.6 Hz, 1H), 7.02 (s, 2H), 6.37 (dd, J=4.8, 7.6 Hz, 1H), 3.62 (s, 2H), 2.68-2.57 (m, 1H), 2.48-2.40 (m, 1H), 2.14-2.05 (m, 5H), 1.87-1.79 (m, 2H), 1.55-1.33 (m, 4H). HPLC Rₜ=2.778 min in 8 min chromatography, purity 99.2%. LCMS Rₜ=1.115 min in 2.5 min chromatography, purity 993%, MS ESI calcd. for 513.26, [M+H]⁺ 514.26, found 514.3.

Step 4. To a solution of (1r,4r)-4-((4-(2-(2-aminopyridin-3-yl)-5-phenyl-3H-imidazo[4,5-b]pyridin-3-yl)benzyl)(methyl)amino)cyclohexane-1-carbonitrile (500 mg, 0.973 mmol) in ethanol (20.0 mL) was added NH₂OH (406 mg, 2.92 mmol, 50% in water) and NaHCO₃ (245 mg, 2.92 mmol). The mixture was stirred at 90° C. for 12 hrs under N₂ atmosphere. The reaction mixture was concentrated. Water (10 mL) was added into the reaction. The resulting mixture was extracted with DCM (15 mL×3). The combined organic phase was washed with brine (1 mL), dried over anhydrous Na₂SO₄, filtered and concentrated to give (1r,4r,Z)-4-((4-(2-(2-aminopyridin-3-yl)-5-phenyl-3H-imidazo[4,5-b]pyridin-3-yl) benzyl)(methyl)amino)-N′-hydroxycyclohexane-1-carboximidamide (500 mg, crude) as a yellow solid, which was used in the next step directly. LCMS Rₜ=0.387 min in 1 min chromatography, purity 76.9%, MS ESI calcd. for 546.29, [M+H]⁺ 547.29. found 547.2.

Step 5. To a solution of (1r,4r,Z)-4-((4-(2-(2-aminopyridin-3-yl)-5-phenyl-3H-imidazo[4,5-b]pyridin-3-yl)benzyl)(methyl)amino)-N′-hydroxycyclohexane-1-carboximidamide (500 mg, 0.915 mmol) in THF (10.00 mL) was added di(1H-imidazol-1-yl)methanethione (244 mg, 1.37 mmol). The mixture was stirred at 25° C. for 1 hr under N₂. Then water (5 mL) was added to the reaction mixture. The resulting mixture was extracted with DCM (20 mL×3). The combined organic phase was washed with brine (20 mL), dried over anhydrous Na₂SO₄, filtered and concentrated. The residue was then dissolved in THF (10 mL). BF₃·Et₂O (0.85 mL, 2.74 mmol) was added into the mixture. The mixture was stirred at 25° C. for 3 hrs under N₂. Water (10 mL) was added to the reaction mixture. The reaction mixture was then adjusted to pH~7 by saturated aqueous NaHCO₃. The resulting mixture was extracted with DCM (20 mL×3). The combined organic phase was washed with brine (20 mL), dried over anhydrous Na₂SO₄, filtered and concentrated. The residue was purified by prep-HPLC (Column: Welch Xtimate C18 150*30 mm*5 um; mobile phase: [water (NH₄HCO₃)-ACN]; B %: 0%-35%; 25 min) and then purified by prep-HPLC (Column: Welch Xtimate C18 150*30 mm*5 um; mobile phase: [water (FA)-ACN]; B %: 10%-40%; 40 min) to afford 3-((1r,4r)-4-((4-(2-(2-aminopyridin-3-yl)-5-phenyl-3H-imidazo[4,5-b]pyridin-3-yl)benzyl)(methyl)amino)cyclohexyl)-1,2,4-thiadiazol-5(4H)-one I-47 (2.20 mg, 0.00354 mmol, 0.39% yield) as a light yellow solid and 3-((1s,4s)-4-((4-(2-(2-aminopyridin-3-yl)-5-phenyl-3H-imidazo[4,5-b]pyridin-3-yl)benzyl)(methyl)amino)cyclohexyl)-1,2,4-thiadiazol-5(4H)-one I-48 (2.60 mg, 0.00423 mmol, 0.46% yield) as alight yellow solid.

I-47: ¹H NMR (DMSO-d₆, 400 MHz,) δ=8.29-8.18 (m, 2H), 8.05-7.97 (m, 4H), 7.50-7.36 (m, 7H), 7.16 (dd, J=1.6, 7.6 Hz, 1H), 7.02 (s, 2H), 6.38 (dd, J=4.8, 7.6 Hz, 1H), 3.66 (s, 2H), 2.48-2.43 (m, 2H), 2.17 (s, 3H), 2.05-1.98 (m, 2H), 1.93-1.87 (m, 2H), 1.50-1.38 (m, 4H). HPLC Rₜ=2.870 min in 8 min chromatography, purity 94.8%. LCMS Rₜ=1.152 min in 2.5 min chromatography, purity 99.3%, MS ESI calcd. for 588.24, [M+H]⁺ 5889.24, found 589.3.

I-48: ¹H NMR (DMSO-d₆, 400 MHz,) δ=8.28 (d, J=8.4 Hz, 1H), 8.13 (s, 1H), 8.04-7.99 (m, 4H), 7.75-7.54 (m, 4H), 7.49-7.44 (m, 2H), 7.43-7.38 (m, 1H), 7.20 (dd, J=1.6, 7.6 Hz, 1H), 6.95 (s, 2H), 6.44 (dd, J=4.8, 7.6 Hz, 1H), 4.51-4.04 (m, 2H), 2.65-2.56 (m, 2H), 2.49-2.42 (m, 3H), 2.11-1.99 (m, 4H), 1.74-1.57 (m, 2H), 1.52-1.41 (m, 2H). HPLC Rₜ=3.030 min in 8 min chromatography, purity 95.8%. LCMS Rₜ=1.190 min in 2.5 min chromatography, purity 94.0%, MS ESI calcd. for 588.24. [M+H]⁺ 589.24, found 589.3.

### Example 41. AKT1 E17K Kinase Biological Activity Assay for Exemplary Compounds

AKT1 E17K kinase assay: Inhibitory effects of compounds on AKT1 E17K were undertaken in a 10-pt dose response kinase activity assay measuring phospho-peptide product formation based on TR-FRET signal as described in the following paragraph.

Purified human recombinant kinase full-length AKT1 E17K was expressed in insect cells and activated in vitro by PDPK1. Ulight™-CREBtide (PerkinElmer, catalog number TRF0107) was used as the peptide substrate. The reaction buffer consisted of 50 mM HEPES pH 7.5, 10 mM MgCl₂, 0.01% Triton X-100, 0.01% BSA, 2 mM DTT, 0.6 nM AKT1 E17K, 50 nM Ulight™-CREBtide and either 50 μM ATP or 2 mM ATP. The compound solution was prepared as 10 mM DMSO stock. For the assay, a 1:3 serial dilution 10-pt dose response of test compounds, in duplicate, for each concentration were dispensed into a 384-well plate (Corning, catalog number 4513). Final DMSO concentration in the assay was 1%. The enzyme mixture (5 μL, 1.2 nM) were added and the mixture was incubated for 15 min at RT prior to the start of the kinase reaction by the addition of 5 μL substrate mixture (100 nM Ulight™-CREBtide). The reaction was stopped after 60 min of incubation at RT by the addition of 10 μL of Detection Mix (PerkinElmer, #CR97-100) consisting of Europium-anti-phospho-CREB (Ser133) (Perkin Elmer, catalog numberTRF0200). Incubation with the detection mix for a further 60 min allowed binding of antibody to the phospho-Ulight™ CREBtide. The fluorescence emissions at 620 nm and 665 nm after excitation at 350 nm was measured using Envision reader. The ratio of the emissions at 665 nm and at 620 nm provided the measure for the amount of phosphorylated CREBtide product of AKT1 E17K kinase activity. To determine the IC₅₀ values, the data was normalized (enzyme reaction without inhibitor=0% inhibition, all other assay components but no enzyme=100% inhibition) and IC₅₀ values were calculated by a 4-parameter fit using an in-house developed protocol.

Table 2 assigns each compound a code for potency in the AKT1 E17K kinase assay: A, B. C, or D. According to the code, A represents an IC₅₀ value <50 nM; B represents an IC₅₀ value from ≥50 nM to <200 nM; C represents an IC₅₀ value from ≥200 nM to <500 nM; and D represents an IC₅₀ value ≥500 nM.

TABLE 2

| Compound | AKT1 E17K Lance |
|---|---|
| No. | IC₅₀ |
| I-1 | B |
| I-2 | C |
| I-3 | D |
| I-4 | B |
| I-5 | B |
| I-6 | C |
| I-7 | D |
| I-8 | D |
| I-9 | B |
| I-10 | A |
| I-11 | A |
| I-12 | C |
| I-13 | C |
| I-14 | C |
| I-15 | B |
| I-16 | C |
| I-17 | C |
| I-18 | A |
| I-19 | C |
| I-20 | A |
| I-21 | B |
| I-22 | D |
| I-23 | D |
| I-24 | D |
| I-25 | A |
| I-26 | A |
| I-27 | B |
| I-28 | D |
| I-29 | D |
| I-38 | D |
| I-39 | C |
| I-40 | A |
| I-41 | B |
| I-42 | B |
| I-43 | D |
| I-44 | D |
| I-45 | D |
| I-46 | D |
| I-47 | D |
| I-48 | B |

### Example 42—AKT1 Kinase Biological Activity Assay for Exemplary Compounds

AKT1 kinase assay: Inhibitory effects of compounds on wildtype AKT1 were undertaken in a 10-pt dose response kinase activity assay measuring phospho-peptide product formation based on TR-FRET signal as described in the following paragraph.

Purified human recombinant kinase full-length wildtype AKT1 was expressed in insect cells and activated in vitro by PDPK1. Ulight™-CREBtide (PerkinElmer, catalog number TRF0107) was used as the peptide substrate. The reaction buffer consisted of 50 mM HEPES pH 7.5, 10 mM MgCl₂, 0.01% Triton X-100, 0.01% BSA, 2 mM DTT, 0.6 nM AKT1, 50 nM Ulight™-CREBtide and either 50 μM ATP or 2 mM ATP. The compound solution was prepared as 10 mM DMSO stock. For the assay, a 1:3 serial dilution 10-pt dose response of test compounds, in duplicate, for each concentration were dispensed into a 384-well plate (Corning, catalog number 4513). Final DMSO concentration in the assay was 1%. The enzyme mixture (5 μL, 1.2 nM) were added and the mixture was incubated for 15 min at RT prior to the start of the kinase reaction by the addition of 5 μL substrate mixture (100 nM Ulight™-CREBtide). The reaction was stopped after 60 min of incubation at RT by the addition of 10 μL of Detection Mix (PerkinElmer. #CR97-100) consisting of Europium-anti-phospho-CREB (Ser133) (Perkin Elmer, catalog numberTRF0200). Incubation with the detection mix for a further 60 min allowed binding of antibody to the phospho-Ulight™ CREBtide. The fluorescence emissions at 620 nm and 665 nm after excitation at 350 nm was measured using Envision reader. The ratio of the emissions at 665 nm and at 620 nm provided the measure for the amount of phosphorylated CREBtide product of AKT1 kinase activity. To determine the IC₅₀ values, the data was normalized (enzyme reaction without inhibitor=0% inhibition, all other assay components but no enzyme=100% inhibition) and IC₅₀ values were calculated by a 4-parameter fit using an in-house developed protocol.

Results are provided in Table 3. The symbol +++ represents an IC₅₀ value <500 nM; the symbol ++ represents an IC₅₀ value >500 nM.

TABLE 3

| Compound | AKT1 Lance |
|---|---|
| No. | IC50 |
| I-1 | +++ |
| I-2 | +++ |
| I-3 | +++ |
| I-4 | +++ |
| I-5 | +++ |
| I-6 | +++ |
| I-7 | +++ |
| I-8 | +++ |
| I-9 | +++ |
| I-10 | +++ |
| I-11 | +++ |
| I-12 | +++ |
| I-13 | +++ |
| I-14 | +++ |
| I-15 | +++ |
| I-16 | +++ |
| I-17 | +++ |
| I-18 | +++ |
| I-19 | +++ |
| I-20 | +++ |
| I-21 | +++ |
| I-22 | ++ |
| I-23 | ++ |
| I-24 | +++ |
| I-25 | +++ |
| I-26 | +++ |
| I-27 | +++ |
| I-28 | ++ |
| I-29 | +++ |
| I-38 | +++ |
| I-39 | +++ |
| I-40 | +++ |
| I-41 | +++ |
| I-42 | +++ |
| I-43 | +++ |
| I-44 | +++ |
| I-45 | +++ |
| I-46 | +++ |
| I-47 | +++ |
| I-48 | +++ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 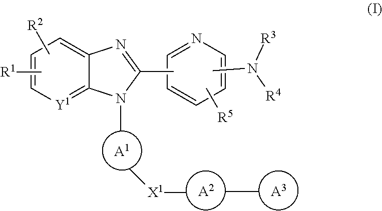
or a pharmaceutically acceptable salt thereof, wherein:
R¹ is phenyl or 6-membered heteroaryl containing 1 or 2 heteroatoms selected from nitrogen, wherein the phenyl and heteroaryl are each substituted with 0, 1, or 2 substituents independently selected from halo or C₁₋₄ alkyl;
R², R³, R⁴, R⁵, and R⁹ are independently hydrogen or C₁₋₄ alkyl;
R⁶ and R⁷ are independently halo or C₁₋₄ alkyl;
R⁸ represents independently for each occurrence hydroxyl, oxo, C₁₋₄ alkoxyl, C₁₋₄ alkyl, C₁₋₄ haloalkyl, cyano, —(C₀₋₄alkylene)-CO₂R⁹, —(C₀₋₄ alkylene)-C(O)N(R¹⁰)(R¹¹), —(C₀₋₄ alkylene)-N(R¹⁰)C(O)R¹², or phenyl, wherein the phenyl is substituted with 0, 1, or 2 substituents independently selected from the group consisting of halo and C₁₋₄ alkyl;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring,
R¹² is C₁₋₆ alkyl or C₃₋₆ cycloalkyl;
A¹ is phenylene or pyridinylene, each of which is substituted with 0 or 1 occurrence of R⁶;
A² is phenalene, C₃₋₆cycloalkyl, or pyridinylene, each of which is substituted with 0 or 1 occurrence of R⁷;
A³ is a 5-membered heteroaryl or heterocyclyl containing 2, 3, or 4 heteroatoms selected from nitrogen and sulphur, wherein the heteroaryl or heterocyclyl is substituted with n occurrences of R⁸;
X¹ is —(C₀₋₃ alkylene)-N(R⁹)C(O)-Ψ, —(C₀₋₃ alkylene)-N(R⁹)-Ψ, -(3-6 membered saturated heterocyclylene containing 1 or 2 heteroatoms selected from nitrogen)-(C₀₋₃ alkylene)-Ψ, or -(3-5 membered saturated heterocyclylene containing 1 or 2 heteroatoms selected from nitrogen)-C(O)-Ψ; wherein Ψ is a bond to A²;
Y¹ is N or —C(H)—; and
n is 0, 1, or 2.

2. The compound of claim 1 or 2, wherein the compound is represented by Formula I: 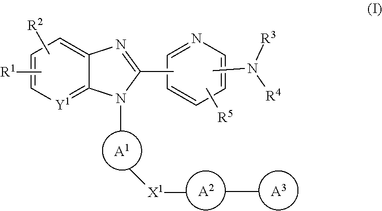
or a pharmaceutically acceptable salt thereof; wherein:
R¹ is phenyl or 6-membered heteroaryl containing 1 or 2 heteroatoms selected from nitrogen, wherein the phenyl and heteroaryl are each substituted with 0, 1, or 2 substituents independently selected from halo or C₁₋₄ alkyl;
R², R³, R⁴, R⁵, and R⁹ are independently hydrogen or C₁₋₄ alkyl;
R⁶ and R⁷ are independently halo or C₁₋₄ alkyl;
R⁸ represents independently for each occurrence hydroxyl, C₁₋₄ alkoxyl, C₁₋₄ alkyl, C₁₋₄ haloalkyl, cyano, —(C₀₋₄ alkylene)-CO₂R⁹, —(C₀₋₄ alkylene)-C(O)N(R¹⁰)(R¹¹), —(C₀₋₄ alkylene)-N(R¹⁰)C(O)R¹², or phenyl, wherein the phenyl is substituted with 0, 1, or 2 substituents independently selected from the group consisting of halo and C₁₋₄ alkyl;
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² is C₁₋₆ alkyl or C₃₋₆ cycloalkyl;
A¹ is phenylene or pyridinylene, each of which is substituted with 0 or 1 occurrence of R⁶;
A² is phenylene or pyridinylene, each of which is substituted with 0 or 1 occurrence of R⁷;
A³ is a 5-membered heteroaryl containing 2, 3, or 4 heteroatoms selected from nitrogen, wherein the heteroaryl is substituted with n occurrences of R⁸;
X¹ is —(C₀₋₃ alkylene)-N(R⁹)C(O)-Ψ or -(3-5 membered saturated heterocyclylene containing 1 or 2 heteroatoms selected from nitrogen)-C(O)-Ψ; wherein Ψ is a bond to A²;
Y¹ is N or —C(H)—; and
n is 0, 1, or 2.

3. The compound of claim 1 or 2, wherein the compound is a compound of Formula I.

4. The compound of claim 1 or 2, wherein the compound is represented by Formula Ia or a pharmaceutically acceptable salt thereof:

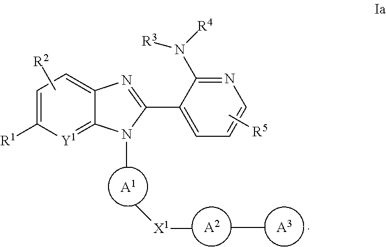

5. The compound of any one of claims 1-4, wherein Y is N.

6. The compound of any one of claims 1-4, wherein Y¹ is —C(H)—.

7. The compound of any one of claims 1-6, wherein R¹ is phenyl.

8. The compound of any one of claims 1-7, wherein R² is hydrogen.

9. The compound of any one of claims 1-8, wherein R³ is hydrogen.

10. The compound of any one of claims 1-9, wherein R⁴ is hydrogen.

11. The compound of any one of claims 1-10, wherein R⁵ is hydrogen.

12. The compound of any one of claims 1-11, wherein A¹ is phenylene substituted with 0 or 1 occurrence of R⁶.

13. The compound of any one of claims 1-11, wherein A¹ is phenylene.

14. The compound of any one of claims 1-11, wherein A¹ is pyridinylene.

15. The compound of any one of claims 1-14, wherein A² is phenylene substituted with 0 or 1 occurrence of R⁷.

16. The compound of any one of claims 1-14, wherein A² is phenylene.

17. The compound of any one of claims 1-14, wherein A² is pyridinylene substituted with 0 or 1 occurrence of R⁷.

18. The compound of any one of claims 1-14, wherein A² is pyridinylene.

19. The compound of any one of claims 1-14, wherein A² is C₃₋₆ cycloalkyl.

20. The compound of any one of claims 1-19, wherein X¹ is —(C₁₋₃ alkylene)-N(R⁹)C(O)-Ψ.

21. The compound of any one of claims 1-19, wherein X¹ is —(C₀₋₃ alkylene)-N(R⁹)-Ψ.

22. The compound of any one of claims 1-19, wherein X¹ is -(3-6 membered saturated heterocyclylene containing 1 or 2 heteroatoms selected from nitrogen)-(C₀₋₃ alkylene)-Ψ.

23. The compound of any one of claims 1-19, wherein X¹ is —(CH₂)—N(R⁹)C(O)-Ψ.

24. The compound of any one of claims 1-19, wherein X¹ is a -(3-5 membered saturated heterocyclylene containing 1 or 2 heteroatoms selected from nitrogen)-C(O)-Ψ.

25. The compound of any one of claims 1-19, wherein X¹ is a -(4-membered saturated heterocyclylene containing 1 heteroatom selected from nitrogen)-C(O)-Ψ.

26. The compound of any one of claims 1-19, wherein X¹ is

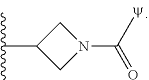 [SMILES: *C1CN(C(C)=O)C1]

27. The compound of any one of claims 1-26, wherein R⁹ is hydrogen.

28. The compound of claim 1 or 2, wherein the compound is a compound of Formula Ib, Ic, Id, Ie, If, or Ig, or a pharmaceutically acceptable salt thereof:

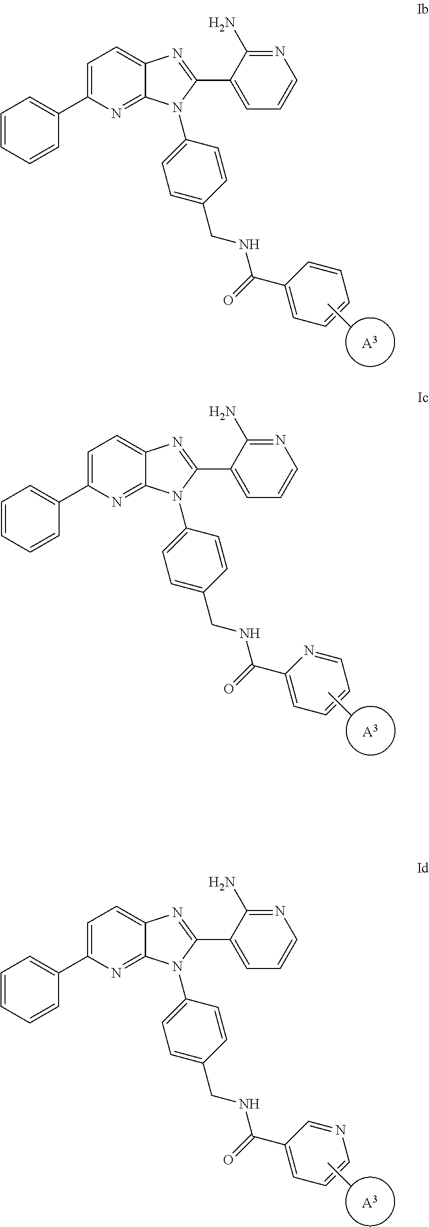

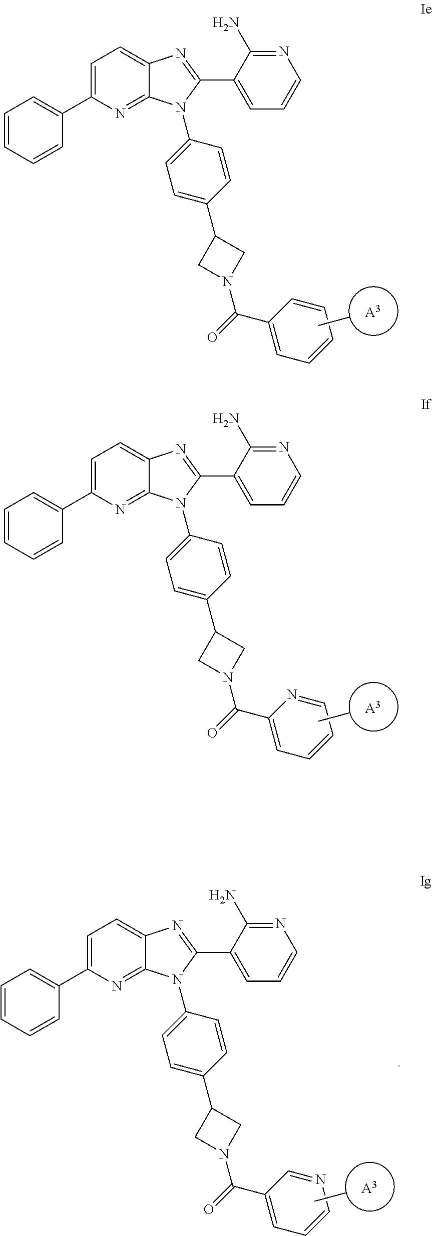

29. The compound of claim 1 or 2, wherein the compound is a compound of Formula Ih, Ii, Ij, Ik, Il, or Im, or a pharmaceutically acceptable salt thereof:

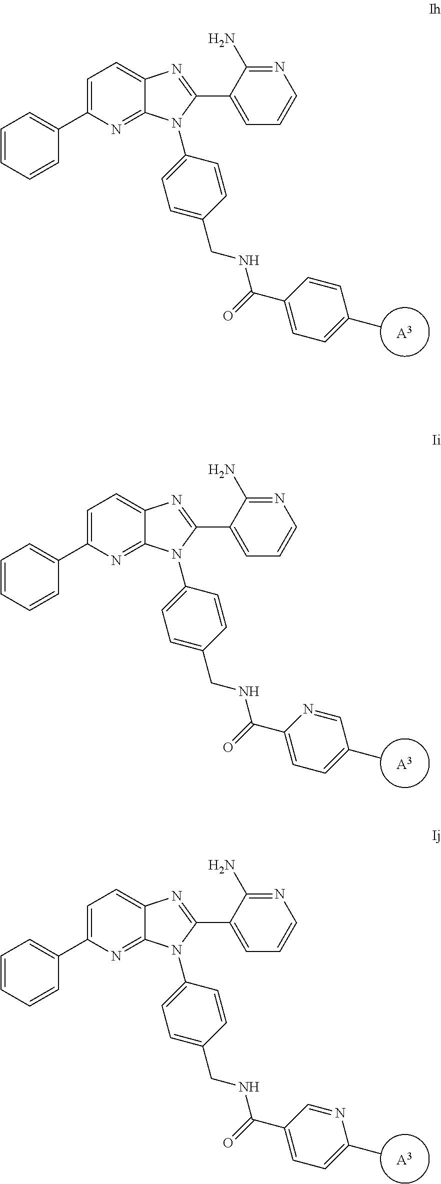

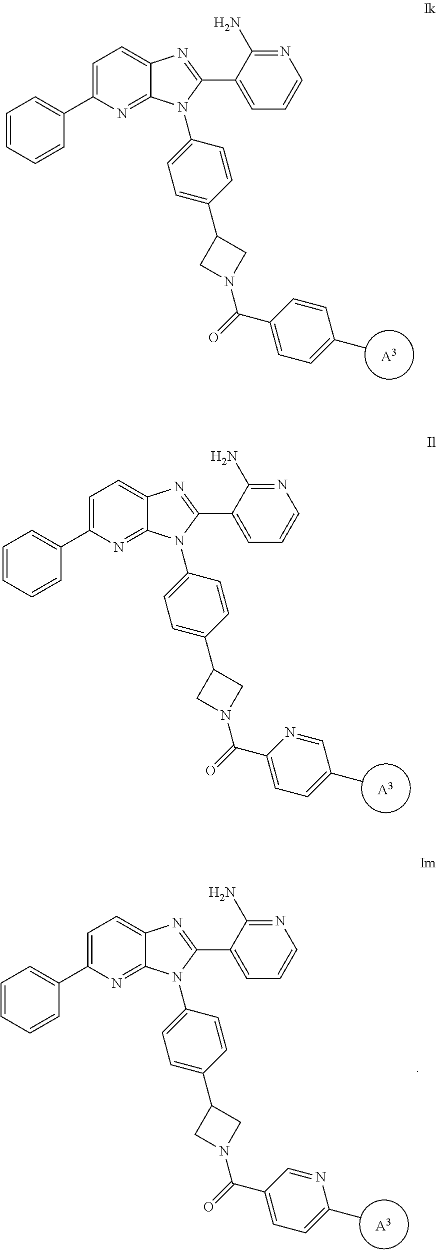

30. The compound of any one of claims 1-29, wherein A³ is a 5-membered heteroaryl or heterocyclyl containing 2 or 3 heteroatoms selected from nitrogen and sulphur, wherein the heteroaryl or heterocyclyl is substituted with n occurrences of R⁸.

31. The compound of any one of claims 1-29, wherein A³ is pyrazolyl substituted with n occurrences of R⁸.

32. The compound of any one of claims 1-29, wherein A³ is 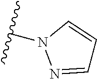 [SMILES: *n1cccn1]
substituted with n occurrences of R⁸.

33. The compound of any one of claims 1-29, wherein A³ is one of the following

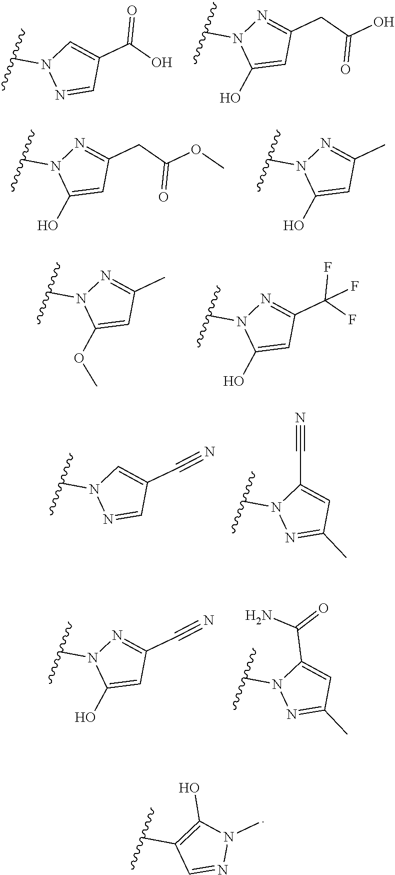

34. The compound of any one of claims 1-29, wherein A³ is tetrazolyl.

35. The compound of any one of claims 1-29, wherein A³ is

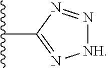 [SMILES: *c1nn[nH]n1]

36. The compound of any one of claims 1-32, wherein n is 1.

37. The compound of any one of claims 1-32, wherein n is 2.

38. The compound of any one of claims 1-32, 36, or 37, wherein R⁸ represents independently for each occurrence hydroxyl, oxo, C₁₋₄ alkoxyl, or C₁₋₄ alkyl.

39. The compound of any one of claims 1-32, 36, or 37, wherein R⁸ represents independently for each occurrence hydroxyl or C₁₋₄ alkyl.

40. The compound of any one of claims 1-32, 36, or 37, wherein R⁸ represents independently for each occurrence hydroxyl or methyl.

41. The compound of any one of claims 1-32, 36, or 37, wherein R⁸ represents independently for each occurrence C₁₋₄ alkoxyl or C₁₋₄ alkyl.

42. The compound of any one of claims 1-32, 36, or 37, wherein R⁸ represents independently for each occurrence methoxy or methyl.

43. The compound of any one of claims 1-32, 36, or 37, wherein R⁸ represents independently for each occurrence C₁₋₄ alkyl, C₁₋₄ haloalkyl, or cyano.

44. The compound of any one of claims 1-32, 36, or 37, wherein R⁸ represents independently for each occurrence hydroxyl or —(C₀₋₄ alkylene)-CO₂R⁹.

45. The compound of any one of claims 1-32, 36, or 37, wherein R⁸ represents independently for each occurrence hydroxyl or —CH₂CO₂CH₃.

46. The compound of any one of claims 1-32 or 36, wherein R⁸ is —CO₂H.

47. A compound in Table 1 herein, or a pharmaceutically acceptable salt thereof.

48. A pharmaceutical composition comprising a compound of any one of claims 1-47 and a pharmaceutically acceptable carrier.

49. A method of treating a disease or disorder associated with aberrant AKT1 signaling, comprising administering to a subject in need thereof a therapeutically effective amount of a compound of any one of claims 1-47 to treat the disease or disorder.

50. The method of claim 49, wherein the disease or disorder associated with aberrant AKT1 signaling is an AKT1 E17K associated disease or disorder.

51. A method of treating cancer, comprising administering to a subject in need thereof a therapeutically effective amount of a compound of any one of claims 1-47 to treat the cancer.

52. The method of claim 51, wherein the cancer is a solid tumor.

53. The method of claim 51, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

54. The method of claim 51, wherein the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, uterine corpus endometrial carcinoma, cervical cancer, endocervical cancer, thyroid carcinoma, prostate adenocarcinoma, skin cutaneous melanoma, bladder urothelial carcinoma, head and neck squamous cell carcinoma, or stomach adenocarcinoma.

55. The method of claim 51, wherein the cancer is an adenocarcinoma, squamous cell carcinoma, epithelial neoplasm, glioma, ductal neoplasm, lobular neoplasm, cystic neoplasm, mucinous neoplasm, or serous neoplasm, acinar cell neoplasm, basal cell neoplasm, fibroepithelial neoplasm, transitional cell papilloma, or transitional cell carcinoma.

56. The method of claim 51, wherein the cancer is a cervical cancer, uterine cancer, breast cancer, thyroid cancer, prostate cancer, lung cancer, bladder cancer, skin cancer, stomach cancer, lymphoma, or leukemia.

57. The method of claim 51, wherein the cancer is a lymphoma or leukemia.

58. The method of any one of claims 51-57, wherein the cancer has an AKT1 mutation.

59. The method of any one of claims 51-57, wherein the cancer has an AKT1 E17K mutation.

60. A method of treating a disease or disorder associated with active PI3K signaling, comprising administering to a subject in need thereof a therapeutically effective amount of a compound of any one of claims 1-47 to treat the disease or disorder.

61. The method of any one of claims 49-60, wherein the subject is a human.

62. A method of inhibiting AKT1 activity, comprising contacting an AKT1 with an effective amount of a compound of any one of claims 1-47 to thereby inhibit the AKT1 activity.

63. The method of claim 62, wherein the AKT1 is AKT1 E17K.
